(19) United States
(12) Patent Application Publication
(10) Pub. No.: US 2024/0415889 A1
(43) Pub. Date: Dec. 19, 2024

(54) MOLECULES THAT BIND TO CD94/NKG2A HETERODIMER POLYPEPTIDES

(71) Applicant: University of Pittsburgh - of the Commonwealth System of Higher Education, Pittsburgh, PA (US)

(72) Inventors: Dimiter Stanchev Dimitrov, Pittsburgh, PA (US); Dusan Baek, Pittsburgh, PA (US); Yae Jin Kim, Pittsburgh, PA (US); John W. Mellors, Pittsburgh, PA (US)

(21) Appl. No.: 18/703,946

(22) PCT Filed: Dec. 13, 2022
(86) PCT No.: PCT/US2022/052652

§ 371 Date: Apr. 23, 2024

Related U.S. Application Data
(60) Provisional application No. 63/289,495, filed on Dec. 14, 2021.

(51) Int. Cl. A61K 35/17; A61K 39/00; A61K 47/68; C07K 16/28; C07K 16/32

(52) U.S. Cl. CPC A61K 35/17; A61K 39/4613; A61K 39/4631; A61K 39/464429; A61K 47/68; C07K 16/2803; C07K 16/2851; C07K 16/32; A61K 2239/13; A61K 2239/17; A61K 2239/21; C07K 2317/31; C07K 2317/55; C07K 2317/622; C07K 2317/75; C07K 2317/92; C07K 2319/02; C07K 2319/03

(57) ABSTRACT

This document provides methods and materials involved in binding a binder (e.g., an antibody, antigen binding fragment, antibody domain, CAR, cell engager, and/or ADC) to a CD94/NKG2A polypeptide. For example, binders (e.g., antibodies, antigen binding fragments, antibody domains, CARs, cell engagers, and/or ADCs) that bind to a CD94/NKG2A polypeptide and methods and materials for using one or more such binding molecules to treat a mammal (e.g., a human) having cancer and/or a viral infection are provided.

### CROSS-REFERENCE TO RELATED APPLICATIONS

This application is a National Stage application under 35 U.S.C. § 371 of International Application No. PCT/US2022/052652, having an International Filing Date of Dec. 13, 2022, which claims the benefit of U.S. Provisional Application Ser. No. 63/289,495, filed Dec. 14, 2021. The disclosure of the prior application is considered part of (and is incorporated by reference in) the disclosure of this application.

### SEQUENCE LISTING

This application contains a Sequence Listing that has been submitted electronically as an XML file named “48881-0033WO1.XML.” The XML file, created on Jun. 9, 2023, is 302,083 bytes in size. The material in the XML file is hereby incorporated by reference in its entirety.

### BACKGROUND

### 1. Technical Field

This document relates to methods and materials involved in binding a molecule (e.g., an antibody, a fragment of an antibody, an antibody domain, a chimeric antigen receptor (CAR), a cell engager, or an antibody-drug conjugate (ADC)) to a CD94/NKG2A heterodimer polypeptide (referred to herein as a CD94/NKG2A polypeptide). For example, this document provides binders (e.g., antibodies, antigen binding fragments, antibody domains, CARs, cell engagers, or ADCs) that bind to a CD94/NKG2A polypeptide and methods and materials for using such binders to treat cancer and/or a viral infection (e.g., an HIV infection). This document also provides cells (e.g., host cells) designed to express one or more binders (e.g., antibodies, antigen binding fragments, antibody domains, CARs, or cell engagers) having the ability to bind to a CD94/NKG2A polypeptide and methods and materials for using such cells to treat cancer and/or a viral infection (e.g., an HIV infection).

### 2. Background Information

CD94/NKG2A is an inhibitory C-type lectin heterodimer receptor that is expressed on 20-60% of natural killer (NK) cells, specifically interacts with human leukocyte antigen (HLA) class I histocompatibility antigen, alpha chain E (HLA-E) (also known as MHC class I antigen E). Ligation of CD94/NKG2A with HLA-E leads to the inhibition of NK cell and CD8⁺ T cell activities through phosphorylation of immunoreceptor tyrosine-based inhibitory motifs (ITIM) of CD94/NKG2A. As the up-regulation of HLA-E expression on tumor cells and HIV-infected lymphocytes were reported, recent studies have shown that tumor cells and HIV-infected lymphocytes obtained the capability to escape from immune cell-mediated clearance through triggering a CD94/NKG2A mediated inhibitory signal.

### SUMMARY

This document provides methods and materials involved in binding a molecule (e.g., an antibody, an antigen binding fragment, an antibody domain, a CAR, a cell engager, or an ADC) to a CD94/NKG2A polypeptide. For example, this document provides binders (e.g., antibodies, antigen binding fragments, antibody domains, CARs, cell engagers, or ADCs) that bind to a CD94/NKG2A polypeptide and methods and materials for using one or more such binders to treat a mammal (e.g., a human) having cancer and/or a viral infection (e.g., an HIV infection).

This document also provides cells (e.g., host cells) designed to express one or more binders (e.g., antibodies, antigen binding fragments, antibody domains, CARs, or cell engagers) having the ability to bind to a CD94/NKG2A polypeptide and methods and materials for using such cells to treat cancer and/or a viral infection (e.g., an HIV infection).

As described herein, binders (e.g., one or more antibodies, one or more antigen binding fragments, one or more antibody domains, one or more CARs, one or more cell engagers, and/or one or more ADCs) can be designed to have the ability to bind to a CD94/NKG2A polypeptide. For example, a binder (e.g., an antibody, an antigen binding fragment, an antibody domain, a CAR, a cell engager, or an ADC) provided herein can have the ability to bind to a polypeptide comprising, consisting essentially of, or consisting of the amino acid sequence of a human CD94/NKG2A polypeptide as set forth in SEQ ID NO:74 and SEQ ID NO:75, SEQ ID NO:256 and SEQ ID NO:257, and/or SEQ ID NO:259 (see, e.g., FIG. 1).

In some cases, a binder provided herein (e.g., one or more antibodies, one or more antigen binding fragments, one or more antibody domains, one or more cell engagers, and/or one or more ADCs) (a) can bind to a CD94/NKG2A polypeptide present on the surface of an NK cell or T cell (e.g., a CD8⁺ T cell) without triggering an inhibitory response within the NK cell or T cell, (b) can, via the binding, block the ability of HLA-E present on a cancer and/or virally infected cell from triggering an inhibitory response within the NK cell or T cell via the CD94/NKG2A polypeptide, and/or (c) can, via the binding, trigger internalization of the CD94/NKG2A polypeptide leading to decreased surface expression of the CD94/NKG2A polypeptide on the NK cell or T cell. For example, a binder (e.g., an antibody, an antigen binding fragment, an antibody domain, a cell engager, or an ADC) provided herein can have the ability to bind to a polypeptide comprising, consisting essentially of, or consisting of the amino acid sequence of a human CD94/NKG2A polypeptide as set forth in SEQ ID NO:74 and SEQ ID NO:75, SEQ ID NO:256 and SEQ ID NO:257, and/or SEQ ID NO:259 (see, e.g., FIG. 1) that is present on the surface of an NK cell or T cell (e.g., a CD8⁺ T cell) in a manner that does not trigger an inhibitory response within the NK cell or T cell and in a manner that blocks the ability of HLA-E present on a cancer and/or virally infected cell from triggering an inhibitory response within the NK cell or T cell.

In some cases, two sets of three CDRs of an antigen binding fragment provided herein (e.g., SEQ ID NOs:1-3 and 9-11 or SEQ ID NOs:17-19 and 25-27) can be engineered into a CAR to create CAR⁺ cells (e.g., CAR⁺ T cells, CAR⁺ stem cells such as CAR⁺ induced pluripotent stem cells, or CAR⁺ NK cells) having the ability to target CD94/NKG2A⁺ cells.

In some cases, two sets of three CDRs of an antigen binding fragment provided herein (e.g., SEQ ID NOs:1-3 and 9-11 or SEQ ID NOs:17-19 and 25-27) can be engineered into an antibody, antigen binding fragment, or antibody domain structure to create a binder (e.g., fully human binder) having the ability to bind to and inhibit the inhibitory activity of a CD94/NKG2A polypeptide of a CD94/NKG2A⁺ NK cell and/or CD94/NKG2A⁺ CD8⁺ T cell.

In some cases, two sets of three CDRs of an antigen binding fragment provided herein (e.g., SEQ ID NOs:1-3 and 9-11 or SEQ ID NOs:17-19 and 25-27) can be engineered into a cell engager such as a bi-specific T cell engager (e.g., a BiTE), a tri-specific T cell engager (e.g., a TriTE), a bi-specific killer engager (e.g., a BiKE), and/or a tri-specific killer engager (e.g., a TriKE) that targets an antigen of interest to create cell engagers having the ability to (a) engage CD94/NKG2A⁺ NK cells via an CD94/NKG2A polypeptide and/or engage a CD94/NKG2A⁺ T cell (e.g., a CD94/NKG2A⁺ CD8⁺ T cell) via an CD94/NKG2A polypeptide and (b) target such NK and/or T cells against cells expressing the target antigen of interest, thereby inducing one or more immune responses (e.g., NK and/or T cell immune responses and/or ADCC using a cell engager in the absence of an Fc-containing antibody) against cells expressing the target antigen of interest. In some cases, a cell engager provided herein (e.g., a BiKE) can bind to NK cells without including an antigen binding domain that binds to CD16. In some cases, a cell engager provided herein (e.g., a BiTE) can bind to T cells without including an antigen binding domain that binds to CD3. It is noted that BiKE- and TriKE-mediated killing can be referred to ADCC even though it is not initiated by an Fc domain.

In addition, as described herein, binders (e.g., one or more antibodies, one or more antigen binding fragments, and/or one or more antibody domains) provided herein can be used to create conjugates that include the binder and a drug. For example, ADCs such as full antibody-drug conjugates, Fab-drug conjugates, and/or antibody domain-drug conjugates can be designed to include an appropriate binder provided herein to create the conjugate. Such conjugates can be used to deliver the drug payload to target cells such as CD94/NKG2A⁺ NK cells and/or CD94/NKG2A⁺ T cells (e.g., CD94/NKG2A⁺ CD8⁺ T cells). In some cases, the drug payload can be a drug that promotes NK cell and/or T cell activities against cancer cells and/or virally infected cells.

As also described herein, binders (e.g., one or more antibodies, one or more antigen binding fragments, one or more antibody domains, one or more cell engagers, and/or one or more ADCs) provided herein can be used to treat a mammal (e.g., a human) having cancer. For example, a mammal (e.g., a human) having cancer can be administered a composition comprising one or more binders (e.g., one or more antibodies, one or more antigen binding fragments, one or more antibody domains, one or more cell engagers, and/or one or more ADCs) described herein (a) to block the ability of HLA-E⁺ cancer cells to inhibit NK and/or T cell immune responses via the interaction of HLA-E with a CD94/NKG2A polypeptide present on the surface of the NK and/or T cell, (b) to induce ADCC against cancer cells by targeting an antigen of interest expressed by the cancer cells within the mammal, and/or (c) to increase the survival duration of the mammal from cancer.

As also described herein, binders (e.g., one or more antibodies, one or more antigen binding fragments, one or more antibody domains, one or more cell engagers, and/or one or more ADCs) provided herein can be used to treat a mammal (e.g., a human) having a viral infection (e.g., an HIV infection). For example, a mammal (e.g., a human) having a viral infection (e.g., an HIV infection) can be administered a composition comprising one or more binders (e.g., one or more antibodies, one or more antigen binding fragments, one or more antibody domains, one or more cell engagers, and/or one or more ADCs) described herein (a) to block the ability of HLA-E⁺ virally infected cells to inhibit NK and/or T cell immune responses via the interaction of HLA-E with a CD94/NKG2A polypeptide present on the surface of the NK and/or T cell, (b) to induce ADCC against virally infected cells by targeting an antigen of interest expressed by the virally infected cells within the mammal, and/or (c) to increase the survival duration of the mammal from a viral infection.

As also described herein, cells (e.g., host cells) can be designed to express one or more binders (e.g., antibodies, antigen binding fragments, antibody domains, CARs, or cell engagers) having the ability to bind to a CD94/NKG2A polypeptide. For example, cells such as T cells (e.g., CTLs), stem cells (e.g., induced pluripotent stem cells), or NK cells can be engineered to express one or more CARs having the ability to bind to a CD94/NKG2A polypeptide. In some cases, such cells (e.g., CD94/NKG2A-specific CAR⁺ T cells or NK cells) can be used to modulate a tumor microenvironment (TME) by killing of subset of NK and/or T cells that secrete inhibitory cytokines and/or promote the inhibition of other immune cells.

In some cases, a binder (e.g., an antibody, antigen binding fragment, and/or antibody domain) provided herein can be used to detect the presence or absence of a CD94/NKG2A polypeptide. For example, a binder (e.g., an antibody, antigen binding fragment, and/or antibody domain) provided herein can be used to determine whether or not a sample (e.g., a biological sample) obtained from a mammal (e.g., a human) contains CD94/NKG2A⁺ cells (e.g., CD94/NKG2A⁺ NK cells and/or CD94/NKG2A⁺ T cells). Having the ability to detect the presence or absence of a CD94/NKG2A polypeptide (e.g., CD94/NKG2A⁺ NK and/or CD94/NKG2A⁺ T cells) can allow clinicians, health professionals, and patients to make better decisions about possible treatment options. For example, detection of an abundance of CD94/NKG2A⁺ NK cells and/or CD94/NKG2A⁺ T cells within a tumor site of a mammal can allow clinicians, health professionals, and patients to select an appropriate anti-cancer treatment that promotes CD94/NKG2A⁺ NK cell and/or CD94/NKG2A⁺ T cell activity. Such treatments that promote CD94/NKG2A⁺ NK cell and/or CD94/NKG2A⁺ T cell activity can include, without limitation, administration of a BiKE, TriTE, TriKE, and/or BiTE provided herein that has the ability to bind to CD94/NKG2A without inhibiting the NK cell or T cell and the ability to bind to a target antigen of interest expressed by the cancer cells.

As described herein, using a binder described herein to block a CD94/NKG2A polypeptide from engaging with HLA-E and/or triggering an inhibitory effect on NK cells and/or T cells can provide an effect method for treating cancer and/or a viral infection (e.g., an HIV infection) by, for example, restoring NK cell- and/or CD8⁺ T cell-mediated cytotoxic activities and/or enhancing immunity of surrounding immune cells.

In general, one aspect of this document features an antibody comprising (or consisting essentially of or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody can comprise the ability to bind to SEQ ID NO:74 and SEQ ID NO:75, SEQ ID NO:256 and SEQ ID NO:257, and/or SEQ ID NO:259. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the light chain variable domain or region of the (i). The light chain variable domain or region can comprise an amino acid sequence having at least 90 percent identity to the amino acid sequence set forth in SEQ ID NO:16. The antibody can comprise the heavy chain variable domain or region of the (ii). The heavy chain variable domain or region can comprise an amino acid sequence having at least 90 percent identity to the amino acid sequence set forth in SEQ ID NO:24. The antibody can comprise the light chain variable domain or region of the (ii). The light chain variable domain or region can comprise an amino acid sequence having at least 90 percent identity to the amino acid sequence set forth in SEQ ID NO:32. The antibody can be a monoclonal antibody. The antibody can be an scFv antibody.

In another aspect, this document features an antigen binding fragment comprising (or consisting essentially of or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antigen binding fragment can comprise the ability to bind to SEQ ID NO:74 and SEQ ID NO:75, SEQ ID NO:256 and SEQ ID NO:257, and/or SEQ ID NO:259. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the light chain variable domain or region of the (i). The light chain variable domain or region can comprise an amino acid sequence having at least 90 percent identity to the amino acid sequence set forth in SEQ ID NO:16. The antigen binding fragment can comprise the heavy chain variable domain or region of the (ii). The heavy chain variable domain or region can comprise an amino acid sequence having at least 90 percent identity to the amino acid sequence set forth in SEQ ID NO:24. The antigen binding fragment can comprise the light chain variable domain or region of the (ii). The light chain variable domain or region can comprise an amino acid sequence having at least 90 percent identity to the amino acid sequence set forth in SEQ ID NO:32. The antigen binding fragment can be monoclonal. The antigen binding fragment can be an Fab.

In another aspect, this document features a chimeric antigen receptor comprising (or consisting essentially of or consisting of) an antigen binding domain, a hinge, a transmembrane domain, and one or more signaling domains, wherein the antigen binding domain comprises an antibody or an antigen-binding fragment. The antibody can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody can comprise the ability to bind to SEQ ID NO:74 and SEQ ID NO:75, SEQ ID NO:256 and SEQ ID NO:257, and/or SEQ ID NO:259. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the light chain variable domain or region of the (i). The light chain variable domain or region can comprise an amino acid sequence having at least 90 percent identity to the amino acid sequence set forth in SEQ ID NO:16. The antibody can comprise the heavy chain variable domain or region of the (ii). The heavy chain variable domain or region can comprise an amino acid sequence having at least 90 percent identity to the amino acid sequence set forth in SEQ ID NO:24. The antibody can comprise the light chain variable domain or region of the (ii). The light chain variable domain or region can comprise an amino acid sequence having at least 90 percent identity to the amino acid sequence set forth in SEQ ID NO:32. The antibody can be a monoclonal antibody. The antibody can be an scFv antibody. The antigen binding fragment can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antigen binding fragment can comprise the ability to bind to SEQ ID NO:74 and SEQ ID NO:75, SEQ ID NO:256 and SEQ ID NO:257, and/or SEQ ID NO:259. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the light chain variable domain or region of the (i). The light chain variable domain or region can comprise an amino acid sequence having at least 90 percent identity to the amino acid sequence set forth in SEQ ID NO:16. The antigen binding fragment can comprise the heavy chain variable domain or region of the (ii). The heavy chain variable domain or region can comprise an amino acid sequence having at least 90 percent identity to the amino acid sequence set forth in SEQ ID NO:24. The antigen binding fragment can comprise the light chain variable domain or region of the (ii). The light chain variable domain or region can comprise an amino acid sequence having at least 90 percent identity to the amino acid sequence set forth in SEQ ID NO:32. The antigen binding fragment can be monoclonal. The antigen binding fragment can be an Fab. The antigen binding domain can comprise a scFv having the ability to bind to a CD94/NKG2A polypeptide. The hinge can comprise a hinge set forth in FIG. 13. The transmembrane domain can comprise a transmembrane domain set forth in FIG. 14. The chimeric antigen receptor can comprise one or more signaling domains set forth in FIG. 15.

In another aspect, this document features a cell comprising a chimeric antigen receptor of the preceding paragraph. The cell can be a T cell, a stem cell, or an NK cell.

In another aspect, this document features a cell engager comprising (or consisting essentially of or consisting of) a first antigen binding domain, a linker, and a second antigen binding domain, wherein the first antigen binding domain comprises an antibody or an antigen-binding fragment. The antibody can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody can comprise the ability to bind to SEQ ID NO:74 and SEQ ID NO:75, SEQ ID NO:256 and SEQ ID NO:257, and/or SEQ ID NO:259. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the light chain variable domain or region of the (i). The light chain variable domain or region can comprise an amino acid sequence having at least 90 percent identity to the amino acid sequence set forth in SEQ ID NO:16. The antibody can comprise the heavy chain variable domain or region of the (ii). The heavy chain variable domain or region can comprise an amino acid sequence having at least 90 percent identity to the amino acid sequence set forth in SEQ ID NO:24. The antibody can comprise the light chain variable domain or region of the (ii). The light chain variable domain or region can comprise an amino acid sequence having at least 90 percent identity to the amino acid sequence set forth in SEQ ID NO:32. The antibody can be a monoclonal antibody. The antibody can be an scFv antibody. The antigen binding fragment can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antigen binding fragment can comprise the ability to bind to SEQ ID NO:74 and SEQ ID NO:75, SEQ ID NO:256 and SEQ ID NO:257, and/or SEQ ID NO:259. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the light chain variable domain or region of the (i). The light chain variable domain or region can comprise an amino acid sequence having at least 90 percent identity to the amino acid sequence set forth in SEQ ID NO:16. The antigen binding fragment can comprise the heavy chain variable domain or region of the (ii). The heavy chain variable domain or region can comprise an amino acid sequence having at least 90 percent identity to the amino acid sequence set forth in SEQ ID NO:24. The antigen binding fragment can comprise the light chain variable domain or region of the (ii). The light chain variable domain or region can comprise an amino acid sequence having at least 90 percent identity to the amino acid sequence set forth in SEQ ID NO:32. The antigen binding fragment can be monoclonal. The antigen binding fragment can be an Fab. The first antigen binding domain can comprise a scFv having the ability to bind to a CD94/NKG2A polypeptide. The first antigen binding domain can be an IgG having the ability to bind to a CD94/NKG2A polypeptide. The linker can comprise a linker set forth in FIG. 10 or FIG. 13. The second antigen binding domain can bind to an antigen of interest. The antigen of interest can be expressed on the surface of a cancer cell or virally infected cell. The antigen of interest can be an antigen of interest selected from the group consisting of an EGFR polypeptide, an HER2 polypeptide, a CEACAM5 polypeptide, a CEACAM7 polypeptide, a CD19 polypeptide, a CD22 polypeptide, a CD274 polypeptide, a CD276 polypeptide, a PSMA polypeptide, a PSCA polypeptide, an ADAM10 polypeptide, a mesothelin polypeptide, a GPC2 polypeptide, a FGFR polypeptide, a VEGFR polypeptide, an IGFR polypeptide, an HIV gp120 polypeptide, an HIV gp160 polypeptide, and a SARS-CoV-2 RBD polypeptide. The second antigen binding domain can be an antigen binding domain set forth in FIG. 20. The second antigen binding domain can comprise SEQ ID NO:263 and 264. The second antigen binding domain can comprise SEQ ID NO:265 and 266. The cell engager can comprise a third antigen binding domain. The third antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The third antigen binding domain is an antigen binding domain set forth in FIG. 19. The third antigen binding domain is an antigen binding domain set forth in FIG. 18.

In another aspect, this document features a nucleic acid comprising (or consisting essentially of or consisting of) a nucleic acid sequence encoding at least part of an antibody or an antigen-binding fragment. The antibody can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody can comprise the ability to bind to SEQ ID NO:74 and SEQ ID NO:75, SEQ ID NO:256 and SEQ ID NO:257, and/or SEQ ID NO:259. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the light chain variable domain or region of the (i). The light chain variable domain or region can comprise an amino acid sequence having at least 90 percent identity to the amino acid sequence set forth in SEQ ID NO:16. The antibody can comprise the heavy chain variable domain or region of the (ii). The heavy chain variable domain or region can comprise an amino acid sequence having at least 90 percent identity to the amino acid sequence set forth in SEQ ID NO:24. The antibody can comprise the light chain variable domain or region of the (ii). The light chain variable domain or region can comprise an amino acid sequence having at least 90 percent identity to the amino acid sequence set forth in SEQ ID NO:32. The antibody can be a monoclonal antibody. The antibody can be an scFv antibody. The antigen binding fragment can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antigen binding fragment can comprise the ability to bind to SEQ ID NO:74 and SEQ ID NO:75, SEQ ID NO:256 and SEQ ID NO:257, and/or SEQ ID NO:259. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the light chain variable domain or region of the (i). The light chain variable domain or region can comprise an amino acid sequence having at least 90 percent identity to the amino acid sequence set forth in SEQ ID NO:16. The antigen binding fragment can comprise the heavy chain variable domain or region of the (ii). The heavy chain variable domain or region can comprise an amino acid sequence having at least 90 percent identity to the amino acid sequence set forth in SEQ ID NO:24. The antigen binding fragment can comprise the light chain variable domain or region of the (ii). The light chain variable domain or region can comprise an amino acid sequence having at least 90 percent identity to the amino acid sequence set forth in SEQ ID NO:32. The antigen binding fragment can be monoclonal. The antigen binding fragment can be an Fab. The nucleic acid sequence can encode the heavy chain variable domain or region of any one of the (i)-(ii). The nucleic acid sequence can encode the light chain variable domain or region of any one of the (i)-(ii). The nucleic acid can be a viral vector. The nucleic acid can be a phagemid.

In another aspect, this document features a nucleic acid comprising (or consisting essentially of or consisting of) a nucleic acid sequence encoding a chimeric antigen receptor described above or a cell engager described above. The nucleic acid can be a viral vector. The nucleic acid can be a phagemid.

In another aspect, this document features a host cell comprising a nucleic acid of either of the two preceding paragraphs.

In another aspect, this document features a host cell that expresses a chimeric antigen receptor described above or a cell engager described above. The host cell can be a T cell, stem cell, or NK cell.

In another aspect, this document features an antibody-drug conjugate (ADC) comprising (or consisting essentially of or consisting of) an antigen binding domain covalently linked to a drug, wherein the antigen binding domain comprises an antibody or an antigen binding fragment. The antibody can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody can comprise the ability to bind to SEQ ID NO:74 and SEQ ID NO:75, SEQ ID NO:256 and SEQ ID NO:257, and/or SEQ ID NO:259. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the light chain variable domain or region of the (i). The light chain variable domain or region can comprise an amino acid sequence having at least 90 percent identity to the amino acid sequence set forth in SEQ ID NO:16. The antibody can comprise the heavy chain variable domain or region of the (ii). The heavy chain variable domain or region can comprise an amino acid sequence having at least 90 percent identity to the amino acid sequence set forth in SEQ ID NO:24. The antibody can comprise the light chain variable domain or region of the (ii). The light chain variable domain or region can comprise an amino acid sequence having at least 90 percent identity to the amino acid sequence set forth in SEQ ID NO:32. The antibody can be a monoclonal antibody. The antibody can be an scFv antibody. The antigen binding fragment can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antigen binding fragment can comprise the ability to bind to SEQ ID NO:74 and SEQ ID NO:75, SEQ ID NO:256 and SEQ ID NO:257, and/or SEQ ID NO:259. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the light chain variable domain or region of the (i). The light chain variable domain or region can comprise an amino acid sequence having at least 90 percent identity to the amino acid sequence set forth in SEQ ID NO:16. The antigen binding fragment can comprise the heavy chain variable domain or region of the (ii). The heavy chain variable domain or region can comprise an amino acid sequence having at least 90 percent identity to the amino acid sequence set forth in SEQ ID NO:24. The antigen binding fragment can comprise the light chain variable domain or region of the (ii). The light chain variable domain or region can comprise an amino acid sequence having at least 90 percent identity to the amino acid sequence set forth in SEQ ID NO:32. The antigen binding fragment can be monoclonal. The antigen binding fragment can be an Fab. The antigen binding domain can comprise a scFv having the ability to bind to a CD94/NKG2A polypeptide. The antigen binding domain can be an IgG having the ability to bind to a CD94/NKG2A polypeptide. The drug can be selected from the group consisting of BMS1166, BMS202, IL-2, and IL-12.

In another aspect, this document features a composition comprising (or consisting essentially of or consisting of) an antibody or an antigen binding fragment. The antibody can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody can comprise the ability to bind to SEQ ID NO:74 and SEQ ID NO:75, SEQ ID NO:256 and SEQ ID NO:257, and/or SEQ ID NO:259. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the light chain variable domain or region of the (i). The light chain variable domain or region can comprise an amino acid sequence having at least 90 percent identity to the amino acid sequence set forth in SEQ ID NO:16. The antibody can comprise the heavy chain variable domain or region of the (ii). The heavy chain variable domain or region can comprise an amino acid sequence having at least 90 percent identity to the amino acid sequence set forth in SEQ ID NO:24. The antibody can comprise the light chain variable domain or region of the (ii). The light chain variable domain or region can comprise an amino acid sequence having at least 90 percent identity to the amino acid sequence set forth in SEQ ID NO:32. The antibody can be a monoclonal antibody. The antibody can be an scFv antibody. The antigen binding fragment can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antigen binding fragment can comprise the ability to bind to SEQ ID NO:74 and SEQ ID NO:75, SEQ ID NO:256 and SEQ ID NO:257, and/or SEQ ID NO:259. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the light chain variable domain or region of the (i). The light chain variable domain or region can comprise an amino acid sequence having at least 90 percent identity to the amino acid sequence set forth in SEQ ID NO:16. The antigen binding fragment can comprise the heavy chain variable domain or region of the (ii). The heavy chain variable domain or region can comprise an amino acid sequence having at least 90 percent identity to the amino acid sequence set forth in SEQ ID NO:24. The antigen binding fragment can comprise the light chain variable domain or region of the (ii). The light chain variable domain or region can comprise an amino acid sequence having at least 90 percent identity to the amino acid sequence set forth in SEQ ID NO:32. The antigen binding fragment can be monoclonal. The antigen binding fragment can be an Fab. The composition can comprise the antibody. The composition can comprise the antigen binding fragment. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a composition comprising (or consisting essentially of or consisting of) a cell engager described above. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a composition comprising (or consisting essentially of or consisting of) a cell described above. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a composition comprising (or consisting essentially of or consisting of) an ADC described above. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a method of treating a mammal having cancer or a viral infection. The method comprises (or consists essentially of or consists of) administering, to the mammal, a composition of any of the four preceding paragraphs. The mammal can be a human. The cancer can be selected from the group consisting of lung cancer, prostate cancer, esophageal cancer, stomach cancer, colorectal cancer, liver cancer, vaginal cancer, and cervical cancer. The number of cancer cells or virally infected cells within the mammal can be reduced following the administering step.

In another aspect, this document features a method of treating a mammal having cancer or a viral infection. The method comprises (or consists essentially of or consists of) (a) administering, to the mammal, the composition of any of those same four preceding paragraphs referenced in the preceding paragraph, and (b) administering, to the mammal, a composition comprising a checkpoint inhibitor. The mammal can be a human. The cancer can be selected from the group consisting of lung cancer, prostate cancer, esophageal cancer, stomach cancer, colorectal cancer, liver cancer, vaginal cancer, and cervical cancer. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab. The number of cancer cells or virally infected cells within the mammal can be reduced following the administering steps (a) and (b).

In another aspect, this document features a method for binding a binding molecule to a CD94/NKG2A polypeptide. The method comprises (or consists essentially of or consists of) contacting the CD94/NKG2A polypeptide with an antibody or an antigen binding fragment. The antibody can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody can comprise the ability to bind to SEQ ID NO:74 and SEQ ID NO:75, SEQ ID NO:256 and SEQ ID NO:257, and/or SEQ ID NO:259. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the light chain variable domain or region of the (i). The light chain variable domain or region can comprise an amino acid sequence having at least 90 percent identity to the amino acid sequence set forth in SEQ ID NO:16. The antibody can comprise the heavy chain variable domain or region of the (ii). The heavy chain variable domain or region can comprise an amino acid sequence having at least 90 percent identity to the amino acid sequence set forth in SEQ ID NO:24. The antibody can comprise the light chain variable domain or region of the (ii). The light chain variable domain or region can comprise an amino acid sequence having at least 90 percent identity to the amino acid sequence set forth in SEQ ID NO:32. The antibody can be a monoclonal antibody. The antibody can be an scFv antibody. The antigen binding fragment can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antigen binding fragment can comprise the ability to bind to SEQ ID NO:74 and SEQ ID NO:75, SEQ ID NO:256 and SEQ ID NO:257, and/or SEQ ID NO:259. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the light chain variable domain or region of the (i). The light chain variable domain or region can comprise an amino acid sequence having at least 90 percent identity to the amino acid sequence set forth in SEQ ID NO:16. The antigen binding fragment can comprise the heavy chain variable domain or region of the (ii). The heavy chain variable domain or region can comprise an amino acid sequence having at least 90 percent identity to the amino acid sequence set forth in SEQ ID NO:24. The antigen binding fragment can comprise the light chain variable domain or region of the (ii). The light chain variable domain or region can comprise an amino acid sequence having at least 90 percent identity to the amino acid sequence set forth in SEQ ID NO:32. The antigen binding fragment can be monoclonal. The antigen binding fragment can be an Fab. The contacting can be performed in vitro. The contacting can be performed in vivo. The contacting can be performed within a mammal by administering the antibody or the antigen binding fragment to the mammal. The mammal can be a human.

In another aspect, this document features a method for binding a binding molecule to a CD94/NKG2A polypeptide. The method comprises (or consists essentially of or consists of) contacting the CD94/NKG2A polypeptide with a chimeric antigen receptor described above, a cell engager described above, or an ADC described above. The contacting can be performed in vitro. The contacting can be performed in vivo. The contacting can be performed within a mammal by administering the chimeric antigen receptor, the cell engager, or the ADC to the mammal. The mammal can be a human.

In another aspect, this document features a composition comprising (or consisting essentially of) an antibody or antibody binding fragment, wherein the antibody or the antibody binding fragment comprises the ability to bind to a CD94/NKG2A polypeptide present on an NK cell or T cell without inhibiting the cytotoxic activity of the NK cell or the T cell. The CD94/NKG2A polypeptide can be a human CD94/NKG2A polypeptide. The antibody or the antibody binding fragment can bind to the NK cell. The antibody or the antibody binding fragment can bind to the T cell. In some cases, the antibody or antigen binding fragment of the composition can be an antibody comprising (or consisting essentially of or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody can comprise the ability to bind to SEQ ID NO:74 and SEQ ID NO:75, SEQ ID NO:256 and SEQ ID NO:257, and/or SEQ ID NO:259. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the light chain variable domain or region of the (i). The light chain variable domain or region can comprise an amino acid sequence having at least 90 percent identity to the amino acid sequence set forth in SEQ ID NO:16. The antibody can comprise the heavy chain variable domain or region of the (ii). The heavy chain variable domain or region can comprise an amino acid sequence having at least 90 percent identity to the amino acid sequence set forth in SEQ ID NO:24. The antibody can comprise the light chain variable domain or region of the (ii). The light chain variable domain or region can comprise an amino acid sequence having at least 90 percent identity to the amino acid sequence set forth in SEQ ID NO:32. The antibody can be a monoclonal antibody. The antibody can be an scFv antibody. In some cases, the antibody or antigen binding fragment of the composition can be an antigen binding fragment comprising (or consisting essentially of or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antigen binding fragment can comprise the ability to bind to SEQ ID NO:74 and SEQ ID NO:75, SEQ ID NO:256 and SEQ ID NO:257, and/or SEQ ID NO:259. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the light chain variable domain or region of the (i). The light chain variable domain or region can comprise an amino acid sequence having at least 90 percent identity to the amino acid sequence set forth in SEQ ID NO:16. The antigen binding fragment can comprise the heavy chain variable domain or region of the (ii). The heavy chain variable domain or region can comprise an amino acid sequence having at least 90 percent identity to the amino acid sequence set forth in SEQ ID NO:24. The antigen binding fragment can comprise the light chain variable domain or region of the (ii). The light chain variable domain or region can comprise an amino acid sequence having at least 90 percent identity to the amino acid sequence set forth in SEQ ID NO:32. The antigen binding fragment can be monoclonal. The antigen binding fragment can be an Fab. This paragraph can be referred to as Paragraph A.

In another aspect, this document features a composition comprising an antibody or antibody binding fragment, wherein the antibody or the antibody binding fragment comprises the ability to bind to a CD94/NKG2A polypeptide present on an NK cell or T cell and to reduce expression of CD94/NKG2A polypeptides on the surface of the NK cell or the T cell. The CD94/NKG2A polypeptide can be a human CD94/NKG2A polypeptide. The antibody or the antibody binding fragment can bind to the NK cell. The antibody or the antibody binding fragment can bind to the T cell. In some cases, the antibody or antigen binding fragment of the composition can be an antibody comprising (or consisting essentially of or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody can comprise the ability to bind to SEQ ID NO:74 and SEQ ID NO:75, SEQ ID NO:256 and SEQ ID NO:257, and/or SEQ ID NO:259. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the light chain variable domain or region of the (i). The light chain variable domain or region can comprise an amino acid sequence having at least 90 percent identity to the amino acid sequence set forth in SEQ ID NO:16. The antibody can comprise the heavy chain variable domain or region of the (ii). The heavy chain variable domain or region can comprise an amino acid sequence having at least 90 percent identity to the amino acid sequence set forth in SEQ ID NO:24. The antibody can comprise the light chain variable domain or region of the (ii). The light chain variable domain or region can comprise an amino acid sequence having at least 90 percent identity to the amino acid sequence set forth in SEQ ID NO:32. The antibody can be a monoclonal antibody. The antibody can be an scFv antibody. In some cases, the antibody or antigen binding fragment of the composition can be an antigen binding fragment comprising (or consisting essentially of or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antigen binding fragment can comprise the ability to bind to SEQ ID NO:74 and SEQ ID NO:75, SEQ ID NO:256 and SEQ ID NO:257, and/or SEQ ID NO:259. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the light chain variable domain or region of the (i). The light chain variable domain or region can comprise an amino acid sequence having at least 90 percent identity to the amino acid sequence set forth in SEQ ID NO:16. The antigen binding fragment can comprise the heavy chain variable domain or region of the (ii). The heavy chain variable domain or region can comprise an amino acid sequence having at least 90 percent identity to the amino acid sequence set forth in SEQ ID NO:24. The antigen binding fragment can comprise the light chain variable domain or region of the (ii). The light chain variable domain or region can comprise an amino acid sequence having at least 90 percent identity to the amino acid sequence set forth in SEQ ID NO:32. The antigen binding fragment can be monoclonal. The antigen binding fragment can be an Fab. This paragraph can be referred to as Paragraph B.

In another aspect, this document features a cell engager comprising (or consisting essentially of) a first antigen binding domain, a linker, and a second antigen binding domain, wherein the first antigen binding domain comprises the ability to bind to a CD94/NKG2A polypeptide present on an NK cell or T cell without inhibiting the cytotoxic activity of the NK cell or the T cell. The first antigen binding domain can comprise (or consist essentially of) a scFv having the ability to bind to the CD94/NKG2A polypeptide. The first antigen binding domain can comprise (or consist essentially of) an IgG having the ability to bind to the CD94/NKG2A polypeptide. In some cases, the first antigen binding domain can comprise (or consist essentially of) an antibody comprising (or consisting essentially of or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody can comprise the ability to bind to SEQ ID NO:74 and SEQ ID NO:75, SEQ ID NO:256 and SEQ ID NO:257, and/or SEQ ID NO:259. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the light chain variable domain or region of the (i). The light chain variable domain or region can comprise an amino acid sequence having at least 90 percent identity to the amino acid sequence set forth in SEQ ID NO:16. The antibody can comprise the heavy chain variable domain or region of the (ii). The heavy chain variable domain or region can comprise an amino acid sequence having at least 90 percent identity to the amino acid sequence set forth in SEQ ID NO:24. The antibody can comprise the light chain variable domain or region of the (ii). The light chain variable domain or region can comprise an amino acid sequence having at least 90 percent identity to the amino acid sequence set forth in SEQ ID NO:32. The antibody can be a monoclonal antibody. The antibody can be an scFv antibody. In some cases, the first antigen binding domain can comprise (or consist essentially of) an antigen binding fragment comprising (or consisting essentially of or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antigen binding fragment can comprise the ability to bind to SEQ ID NO:74 and SEQ ID NO:75, SEQ ID NO:256 and SEQ ID NO:257, and/or SEQ ID NO:259. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the light chain variable domain or region of the (i). The light chain variable domain or region can comprise an amino acid sequence having at least 90 percent identity to the amino acid sequence set forth in SEQ ID NO:16. The antigen binding fragment can comprise the heavy chain variable domain or region of the (ii). The heavy chain variable domain or region can comprise an amino acid sequence having at least 90 percent identity to the amino acid sequence set forth in SEQ ID NO:24. The antigen binding fragment can comprise the light chain variable domain or region of the (ii). The light chain variable domain or region can comprise an amino acid sequence having at least 90 percent identity to the amino acid sequence set forth in SEQ ID NO:32. The antigen binding fragment can be monoclonal. The antigen binding fragment can be an Fab. The linker can comprise (or consist essentially of) a linker set forth in FIG. 10 or FIG. 13. The second antigen binding domain can bind to an antigen of interest. The antigen of interest can be expressed on the surface of a cancer cell or virally infected cell. The antigen of interest can be an antigen of interest selected from the group consisting of an EGFR polypeptide, an HER2 polypeptide, a CEACAM5 polypeptide, a CEACAM7 polypeptide, a CD19 polypeptide, a CD22 polypeptide, a CD274 polypeptide, a CD276 polypeptide, a PSMA polypeptide, a PSCA polypeptide, an ADAM10 polypeptide, a mesothelin polypeptide, a GPC2 polypeptide, a FGFR polypeptide, a VEGFR polypeptide, an IGFR polypeptide, an HIV gp120 polypeptide, an HIV gp160 polypeptide, and a SARS-CoV-2 RBD polypeptide. The second antigen binding domain can be an antigen binding domain set forth in FIG. 20, FIG. 17B, FIG. 17C, FIG. 17D, or FIG. 17E. The second antigen binding domain can comprise (or consist essentially of) SEQ ID NO:263 and 264. The second antigen binding domain can comprise (or consist essentially of) SEQ ID NO:265 and 266. The cell engager can comprise a third antigen binding domain. The third antigen binding domain can bind to a polypeptide expressed on the surface ofNK cells. The polypeptide expressed on the surface of NK cells can be a CD16a, NKG2A, NKG2D, NKp30, NKp44, or NKp46 polypeptide. The third antigen binding domain can be an antigen binding domain set forth in FIG. 19. This paragraph can be referred to as Paragraph C.

In another aspect, this document features a method for binding a molecule to a CD94/NKG2A polypeptide on the surface of an NK cell or a T cell without inhibiting the cytotoxic activity of the NK cell or the T cell. The method comprises (or consists essentially of) contacting the NK cell or the T cell with a composition of any of Paragraph A or a cell engager of any of Paragraph C. The method can be in vitro. The method can be in vivo. The method can be in vivo within a mammal. The mammal can be a human.

In another aspect, this document features a method for reducing expression of CD94/NKG2A polypeptides on the surface of a NK cell or a T cell. The method comprises (or consists essentially of) contacting the NK cell or the T cell with a composition of any of Paragraph B or a cell engager of any of Paragraph C. The method can be in vitro. The method can be in vivo. The method can be in vivo within a mammal. The mammal can be a human.

In another aspect, this document features a method for killing HER2⁺ cancer cells. The method comprises (or consists essentially of) contacting immune cells positive for CD94/NKG2A polypeptides with (a) a composition of any of Paragraph A, a composition of any of Paragraph B, or a cell engager of any of Paragraph C, and (b) an anti-HER2 antibody. The method can be in vitro. The method can be in vivo. The method can be in vivo within a mammal. The mammal can be a human. The anti-HER2 antibody can be Pertuzumab.

In another aspect, this document features a method treating a mammal having cancer. The method comprises (or consists essentially of) administering a composition of any of Paragraph A, a composition of any of Paragraph B, or a cell engager of any of Paragraph C to the mammal. The mammal can be a human. The cancer can be a HER2⁺ cancer. The method can further comprise administering an anti-HER2 antibody to the mammal. The anti-HER2 antibody can be Pertuzumab.

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts amino acid residues 1 to 179 of a human CD94 (SEQ ID NO:74) and 1 to 233 of a human NKG2A polypeptide (SEQ ID NO:75). The underlined and bolded amino acid sequence (residues 57 to 179) of this human CD94 (SEQ ID NO:256) and (residues 113-233) of this human NKG2A polypeptide (SEQ ID NO:257) depicts the extracellular domains. FIG. 1 also depicts amino acid residues 1 to 231 of a human NKG2C polypeptide (SEQ ID NO:260). The underlined and bolded amino acid sequence of this human this human NKG2C polypeptide (SEQ ID NO:261) depicts the extracellular domain. To identify CD94/NKG2A binders, SEQ ID NO:256 was linked to SEQ ID NO:257 using a linker (SEQ ID NO:258) between the CD94 and NKG2A sequences to create SEQ ID NO:259. To identify CD94/NKG2A binders, SEQ ID NO:256 was linked to SEQ ID NO:261 using a linker (SEQ ID NO:258) between the CD94 and NKG2C sequences to create SEQ ID NO:262.

FIGS. 2A and 2B depict the amino acid sequences of the heavy chain variable domain (FIG. 2A) and the light chain variable domain (FIG. 2B) of an Fab designated Clone #1 (1B2). The CDRs, framework sequences, and constant domains of each also are provided and delineated.

FIGS. 3A and 3B depict the amino acid sequences of the heavy chain variable domain (FIG. 3A) and the light chain variable domain (FIG. 3B) of an Fab designated Clone #2 (1B2-6). The CDRs, framework sequences, and constant domains of each also are provided and delineated.

FIG. 4 depicts the nucleic acid sequences encoding the indicated chains/domains of Clones #1-#2.

FIG. 5 depicts the structure of exemplary Ig molecules and provides the amino acid and nucleic acid sequences of an exemplary hinge, CH2, and CH3 regions/domains and complete Ig's.

FIG. 6A depicts the structure of exemplary scFv's. FIGS. 6B and 6C depict the amino acid sequences of an exemplary heavy chain variable domain (FIG. 6B) and an exemplary light chain variable domain (FIG. 6C) of an exemplary scFv. The CDRs and framework sequences of each also are delineated. An exemplary linker amino acid sequence such as a linker amino acid sequence set forth in FIG. 10 can be used to link the heavy chain variable domain and the light chain variable domain together to form a scFv. FIGS. 6D-6G depict the structures of exemplary scFv's and provide the amino acid sequences with the linker, CDRs, and framework sequences delineated.

FIGS. 7A and 7B depict the amino acid sequences of an exemplary heavy chain variable domain (FIG. 7A) and an exemplary light chain variable domain (FIG. 7B) of an exemplary scFv. The CDRs and framework sequences of each also are delineated. An exemplary linker amino acid sequence such as a linker amino acid sequence set forth in FIG. 10 can be used to link the heavy chain variable domain and the light chain variable domain together to form a scFv. FIG. 7C depicts the structures of exemplary scFv's and provides the amino acid sequences with the linkers, CDRs, and framework sequences delineated.

FIGS. 8A and 8B depict the amino acid sequences of an exemplary heavy chain variable domain (FIG. 8A) and an exemplary light chain variable domain (FIG. 8B) of an exemplary scFv. The CDRs and framework sequences of each also are delineated. An exemplary linker amino acid sequence such as a linker amino acid sequence set forth in FIG. 10 can be used to link the heavy chain variable domain and the light chain variable domain together to form a scFv.

FIGS. 9A and 9B depict the amino acid sequences of an exemplary heavy chain variable domain (FIG. 9A) and an exemplary light chain variable domain (FIG. 9B) of an exemplary scFv. The CDRs and framework sequences of each also are delineated. An exemplary linker amino acid sequence such as a linker amino acid sequence set forth in FIG. 10 can be used to link the heavy chain variable domain and the light chain variable domain together to form a scFv.

FIG. 10 depicts exemplary linker amino acid sequences that can be used to link a heavy chain variable domain and a light chain variable domain together to form a scFv. These linker sequences also can be used to create CARs and cell engagers.

FIG. 11A depicts the structure of an exemplary CARs. FIG. 11B is a schematic of an exemplary CAR construct designed to express a CAR. A promotor sequence (e.g., a CMV immediate early promotor sequence) can be followed by a signal peptide sequence (e.g., a GM-CSF signal peptide sequence), followed by a scFv provided herein (e.g., a scFv designed to include two sets of three CDRs such as CDR1, CDR2, and CDR3 of a heavy chain and CDR1, CDR2, and CDR3 of a light chain (in either order) of an antigen binding fragment provided herein, for example, SEQ ID NOs:1-3 and 9-11 or SEQ ID NOs:17-19 and 25-27), followed by an optional linker (not shown), followed by an optional hinge (e.g., a CD8 hinge sequence; not shown), followed by a transmembrane sequence (e.g., a CD8 transmembrane sequence), followed by one or more intracellular signaling domain sequences (e.g., a 4-1BB (CD137) intracellular signaling domain sequence and a CD3ζ (intracellular signaling domain sequence).

FIG. 12 depicts the amino acid sequences of exemplary signal peptides that can be used to design a CAR.

FIG. 13 depicts the amino acid sequences of exemplary hinges that can be used to design a CAR.

FIG. 14 depicts the amino acid sequences of exemplary transmembrane domains that can be used to design a CAR.

FIG. 15 depicts the amino acid sequences of exemplary intracellular signaling domains that can be used to design a CAR.

FIG. 16A depicts an amino acid sequence of a CAR (CAR #1) designed to include a scFv created using the CDRs of the Clone #1 Fab. The various components of this CAR (e.g., domains and linkers) are provided and delineated. FIG. 16B depicts an amino acid sequence of a CAR (CAR #2) designed to include a scFv created using the CDRs of the Clone #2 Fab. The various components of this CAR (e.g., domains and linkers) are provided and delineated.

FIG. 17A is a schematic of exemplary BiTEs or BiKEs designed using CDR1, CDR2, and CDR3 of a heavy chain provided herein and CDR1, CDR2, and CDR3 of a light chain provided herein in an Ig format (e.g., an IgG1 format). An anti-CEACAM5 scFv (or any binder targeting an antigen of interest) can be linked to the C-terminus of the light chain via an optional linker (e.g., a (G₄S)₃ linker (SEQ ID NO: 78). FIG. 17B depicts an amino acid sequence of an anti-CEACAM5 scFv sequence, which can be attached to a light chain as shown in FIG. 17A via an optional linker (e.g., a (G₄S)₃ linker (SEQ ID NO: 78). FIG. 17B also depicts a nucleic acid sequence encoding that scFv. FIG. 17C depicts an amino acid sequence of an anti-HER2 scFv sequence, which can be attached to a light chain as shown in FIG. 17A via an optional linker (e.g., a (G₄S)₃ linker (SEQ ID NO: 78). FIG. 17C also depicts a nucleic acid sequence encoding that scFv. FIG. 17D depicts an amino acid sequence of an anti-EGFR scFv sequence, which can be attached to a light chain as shown in FIG. 17A via an optional linker (e.g., a (G₄S)₃ linker (SEQ ID NO: 78). FIG. 17D also depicts a nucleic acid sequence encoding that scFv. FIG. 17E depicts an amino acid sequence of an anti-HIV binder sequence, which can be attached to a light chain as shown in FIG. 17A via an optional linker (e.g., a (G₄S)₃ linker (SEQ ID NO: 78). FIG. 17E also depicts a nucleic acid sequence encoding that binder.

FIG. 18 depicts the amino acid sequences of exemplary antigen binding domains that can be used to design cell engagers (e.g., TriTEs) that bind to T cells.

FIG. 19 depicts the amino acid sequences of exemplary antigen binding domains that can be used to design cell engagers (e.g., TriKEs) that bind to NK cells.

FIG. 20 depicts exemplary amino acid sequences of exemplary antigen binding domains that can be used to design cell engagers (e.g., BiTEs, BiKEs, TriTEs, and/or TriKEs) that bind to the indicated target antigen of interest.

FIG. 21 depicts the amino acid sequence of an exemplary BiTE and/or BiKE cell engagers that include the CDRs of Clone #1 or Clone #2 and target an EGFR polypeptide in addition to binding to a CD94/NKG2A polypeptide.

FIG. 22 depicts schematics of different formats for exemplary cell engagers (e.g., BiTEs or BiKEs) designed to include CDR1, CDR2, and CDR3 of a heavy chain provided herein and/or CDR1, CDR2, and CDR3 of a light chain provided herein.

FIG. 23 contains graphs plotting binding of 1B2 (Fab Clone #1) and of 2A8 (negative control Fab) to a recombinant CD94/NKG2A polypeptide or a CD94/NKG2C polypeptide.

FIG. 24 contains a flow cytometry analysis of 1B2 (Fab Clone #1) and of 2A8 (a negative control Fab) binding to primary NK cells. AAG refers to a human IgG1 Fc having L234A, L235A, and P329G mutations.

FIG. 25 contains graphs plotting equilibrium dissociation constants (K_{D}) from a BLItz Biolayer Interferometry (BLI) system for 1B2 (Fab Clone #1) and a monalizumab analogue (Mona analog). LALA-PG also refers to a human IgG1 Fc having L234A, L235A, and P329G mutations.

FIG. 26 contains graphs plotting a competitive ELISA binding assessment of (1) IgG of Clone #1, (2) a monalizumab analogue, and (3) an isotype control as competitors of binding of a HLA-E tetramer to either a CD94/NKG2A polypeptide or a CD94/NKG2C polypeptide.

FIG. 27 contains graphs plotting binding of 1B2 (IgG Clone #1) and of 1B2-6 (IgG Clone #2) to a CD94/NKG2A polypeptide or a CD94/NKG2C polypeptide.

FIG. 28 contains a graph plotting a competitive ELISA binding assessment of (1) IgG of Clone #1, (2) IgG of Clone #2, (3) monalizumab analog, and (4) an isotype control as competitors of binding of an HLA-E tetramer to a CD94/NKG2A polypeptide.

FIG. 29 contains a flow cytometric binding analysis of IgG Clone #1, IgG Clone #2, or monalizumab analogue (Mona analog) with primary NK cells. The control is secondary antibody only (2^{nd} only).

FIG. 30 contains graphs plotting the binding of IgG Clone #1, IgG Clone #2, or monalizumab analogue (Mona analog) antibodies (10 nM) to NKG2A-positive primary NK cells and NKG2A-negative Farage cells. The control is secondary antibody only (2^{nd} only).

FIG. 31 contains graphs plotting NKG2A expression levels of IL-2-activated NK cells alone or together with HLA-E⁺ cancer cells after treatment with the indicated antibodies (100 nM) for 24 hours.

FIG. 32 contains graphs plotting IFNγ and granzyme B (GrzB) secretion of IL-2-activated NKG2A⁺ NK cells alone or together with HLA-E+ cancer cells after treatment with the indicated antibodies (100 nM) 24 hours using intracellular staining.

FIG. 33 contains a graph plotting the binding of a bi-specific NK cell engager (BINK) designed to bind a CD94/NKG2A polypeptide and a CEACAM5 target antigen of interest. Recombinant polypeptides, (1) a CD94/NKG2A polypeptide, (2) a CD94/NKG2C polypeptide, (3) a CEACAM5-A3B3 domain, and (4) a CEACAM6-AB domain, were used to test binding and specificity.

FIG. 34A is a schematic of an exemplary bi-specific NK cell engager (BiNK) designed to bind simultaneously to a CD94/NKG2A polypeptide and a HER2 target antigen of interest (referred to as “BiNK (anti-HER2×anti-NKG2A)”). As shown, BiNK (anti-HER2×anti-NKG2A) simultaneously binds (a) a biotinylated CD94/NKG2A-Fc fusion polypeptide immobilized to a streptavidin-coated sensor and (b) a HER2 polypeptide. FIG. 34B is a graph plotting BLItz assay results showing loading of the biotinylated CD94/NKG2A-Fc fusion polypeptide followed by binding of BiNK (anti-HER2×anti-NKG2A) followed by binding of the HER2 polypeptide.

FIG. 35A is a graph plotting binding of (a) an exemplary bi-specific NK cell engager (BiNK) designed to bind simultaneously to a CD94/NKG2A polypeptide and a HER2 target antigen of interest (referred to as “BiNK (anti-HER2×anti-NKG2A)”; 100 nM), (b) an exemplary bi-specific NK cell engager (BiNK) designed to bind simultaneously to a CD94/NKG2A polypeptide and a EGFR target antigen of interest (referred to as “BiNK (anti-EGFR×anti-NKG2A)”; 100 nM), and (c) anti-CD94/NKG2A Clone #2 as an IgG molecule (referred to as “Clone #2 IgG (anti-NKG2A)”; 100 nM) to primary NK cells, which are NKG2A⁺, as compared to the binding of an hIgG1 isotype control (100 nM). BiNK (anti-HER2×anti-NKG2A), BiNK (anti-EGFR×anti-NKG2A), and Clone #2 IgG (anti-NKG2A) each exhibited binding to primary NK cells above that observed for the isotype control. FIG. 35B is a graph plotting binding of (a) 100 nM of BiNK (anti-HER2×anti-NKG2A), (b) 100 nM of BiNK (anti-EGFR×anti-NKG2A), and (c) 100 nM of Cetuximab (a chimeric monoclonal antibody that binds to EGFR) to either A549 cells (which are non-small cell lung cancer (NSCLC) cells that are EGFR⁺ and HER2⁺) or Farage cells (which are EGFR⁻, HER2⁻, and NKG2A⁻) as compared to the binding of an hIgG1 isotype control (100 nM). BiNK (anti-HER2×anti-NKG2A), BiNK (anti-EGFR×anti-NKG2A), and Cetuximab each exhibited binding to A549 cells above that observed for the isotype control, while BiNK (anti-HER2×anti-NKG2A), BiNK (anti-EGFR×anti-NKG2A), and Cetuximab exhibited similar binding to Farage cells as that observed for the isotype control. The x-axis for the graphs is Alexa647 intensity, and the y-axis for the graphs is cell count.

FIG. 36 contains graphs plotting the percent cytotoxicity observed using an LDH release assay for primary NK cells (effector cells) incubated with A549 or 293T target cells (E:T ratio=5:1) in the presence of the indicated amounts of (a) BiNK (anti-EGFR×anti-NKG2A), (b) BiNK (anti-HER2×anti-NKG2A), (c) Cetuximab, (d) Pertuzumab (an antibody that binds to HER2), or (e) an hIgG1 isotype control. Use of BiNK (anti-EGFR×anti-NKG2A), BiNK (anti-HER2×anti-NKG2A), Cetuximab, and Pertuzumab resulted in increased NK cell killing of A549 target cells as compared to that observed for the isotype control, while use of Cetuximab resulted in increased NK cell killing of 293T target cells as compared to that observed for the isotype control.

FIG. 37 contains graphs plotting the CD16A-positive, NKG2A-positive, and NKG2C-positive populations (percentage after normalization to vehicle treatment) of CD56-positive NK cells after treatment with 50 IU/mL IL-2 and 100 nM of (a) BiNK (anti-EGFR×anti-NKG2A) referred to as “BiNK” in the graphs of FIG. 37, (b) Clone #2 IgG (anti-NKG2A) referred to as “Clone #2” in the graphs of FIG. 37, and (c) Cetuximab, in a co-culture with A549 cells for 24 hours. Each symbol represents the value obtained from individual healthy donors. * p<0.05; **p<0.01; and *** p<0.001. The percentage of NKG2A-positive cells that were CD56-positive NK cells was reduced when the NK cells were treated with BiNK (anti-EGFR×anti-NKG2A) or Clone #2 IgG (anti-NKG2A) as compared to vehicle.

FIG. 38A is a schematic of a treatment schedule for performing cell killing assays using two administrations. FIG. 38B is a graph plotting the percent cytotoxicity observed using an LDH release assay for primary NK cells (effector cells) incubated with A549 target cells (E:T ratio=2:1) in the presence of (a) vehicle, (b) BiNK (anti-HER2×anti-NKG2A), (c) Clone #2 IgG (anti-NKG2A) referred to as “Clone #2” in the graph of FIG. 38B, or (d) Pertuzumab (Ptz) at the indicated concentrations starting on day 0 followed by vehicle or Pertuzumab (Ptz) at the indicated concentrations on day 1.

FIG. 39 is a graph plotting NKG2A expression levels (fold change of mean fluorescence intensity (MFI)) of IL-2 (50 IU/mL) activated NK cells in co-culture with A549 cells and treated with (a) vehicle, (b) 100 nM of Cetuximab, (c) 100 nM of Clone #2 IgG (anti-NKG2A), which is bivalent and referred to as “Clone #2” in the graph of FIG. 39, (d) 100 nM of BiNK (anti-EGFR×anti-NKG2A) referred to as “BiNK” in the graph of FIG. 39, (e) 100 nM of Clone #2 Fab (anti-NKG2A), which is monovalent referred to as “Clone #2 Fab” in the graph of FIG. 39, (f) 100 nM of Clone #2 IgG (anti-NKG2A) mixture with SA (streptavidin), which is octavalent and referred to as “Clone #2-SA mix” in the graph of FIG. 39, or (g) 100 nM of Clone #2 IgG (anti-NKG2A) mixture with SA (streptavidin) bead, which is multivalent and referred to as “Clone #2-SA bead mix” in the graph of FIG. 39, for 24 hours. Each symbol represents the value obtained from individual healthy donors. **p<0.01.

FIG. 40 contains graphs plotting the percentage of IFNTγ⁺, TNFα⁺, or Granzyme B⁺ (GrzB⁺) cells that are NK cells (normalized to vehicle) 24 hours after IL-2 activation (50 IU/mL), co-culture with A549 cells, and treatment with (a) 100 nM of Cetuximab, (b) 100 nM of Clone #2 IgG (anti-NKG2A), which is bivalent and referred to as “Clone #2” in the graph of FIG. 40, (c) 100 nM of BiNK (anti-EGFR×anti-NKG2A) referred to as “BiNK” in the graph of FIG. 40, (d) 100 nM of Clone #2 Fab (anti-NKG2A), which is monovalent referred to as “Clone #2 Fab” in the graph of FIG. 40, (e) 100 nM of Clone #2 IgG (anti-NKG2A) mixture with SA (streptavidin), which is octavalent and referred to as “Clone #2-SA mix” in the graph of FIG. 40, or (f) 100 nM of Clone #2 IgG (anti-NKG2A) mixture with SA (streptavidin) bead, which is multivalent and referred to as “Clone #2-SA bead mix” in the graph of FIG. 40. Each symbol represents the value obtained from individual healthy donors. **p<0.01; ***p<0.001; and ****p<0.0001.

### DETAILED DESCRIPTION

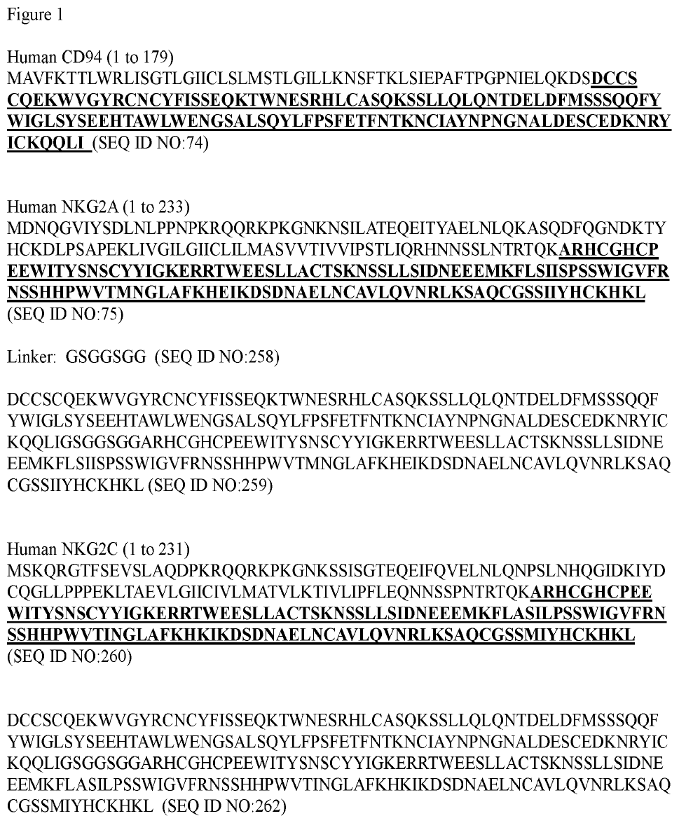

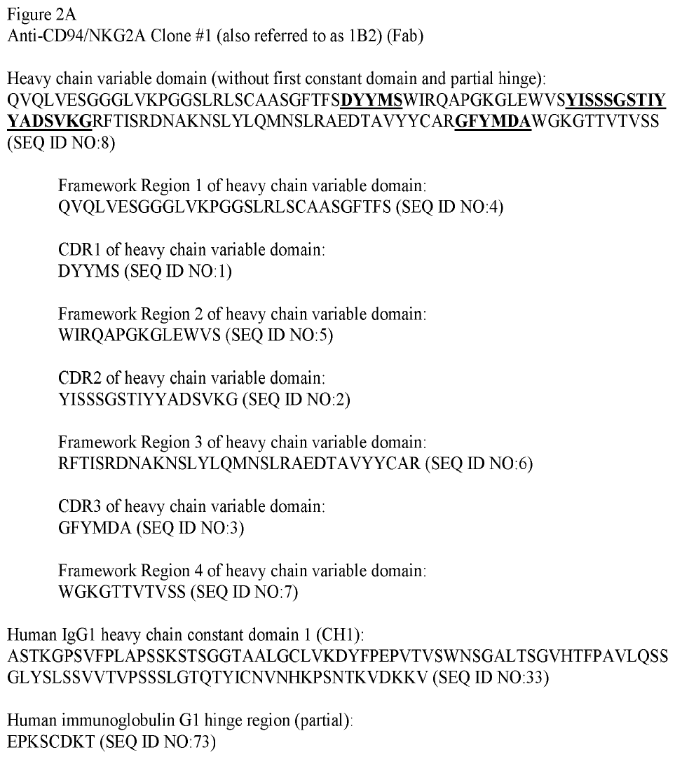

This document provides binders (e.g., antibodies, antigen binding fragments, antibody domains, CARs, cell engagers, and ADCs) that bind (e.g., specifically bind) to a CD94/NKG2A polypeptide (e.g., a human CD94/NKG2A polypeptide). For example, the document provides binders (e.g., antibodies, antigen binding fragments, antibody domains, CARs, cell engagers, and ADCs) that bind (e.g., specifically bind) to a polypeptide comprising, consisting essentially of, or consisting of the amino acid set forth in SEQ ID NO:74 and SEQ ID NO:75, SEQ ID NO:256 and SEQ ID NO:257, and/or SEQ ID NO:259 (see, e.g., FIG. 1). In some cases, a binder (e.g., an antibody, an antigen binding fragment, an antibody domain, a CAR, a cell engager, or an ADC) provided herein can have the ability to bind to a CD94/NKG2A polypeptide and can lack the ability to bind to a CD94/NKG2C heterodimeric polypeptide (e.g., SEQ ID NO:262). For example, a binder (e.g., an antibody, an antigen binding fragment, an antibody domain, a CAR, a cell engager, or an ADC) provided herein can have the ability to bind to a human CD94/NKG2A polypeptide and can lack the ability to bind to a human CD94/NKG2C polypeptide (e.g., SEQ ID NO:262).

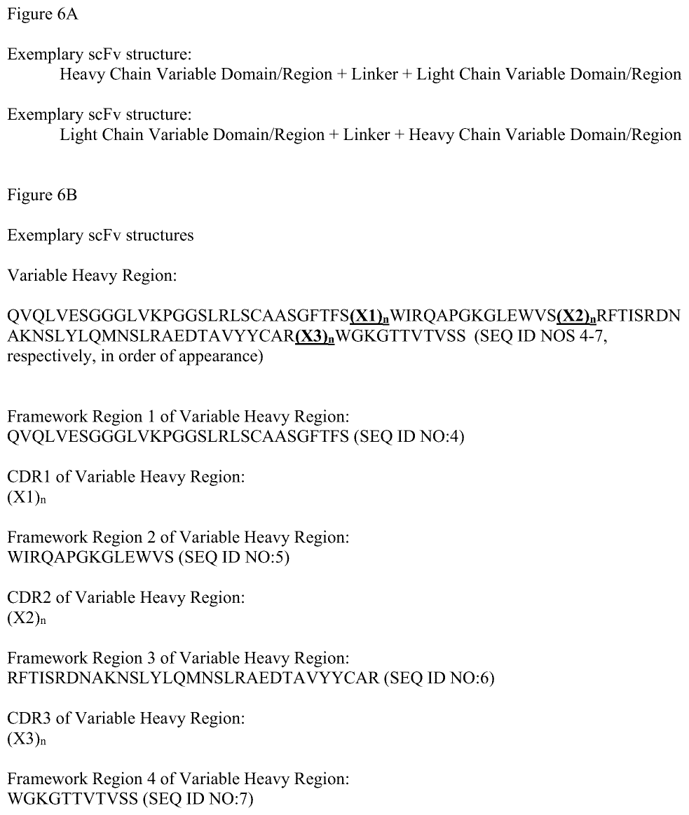

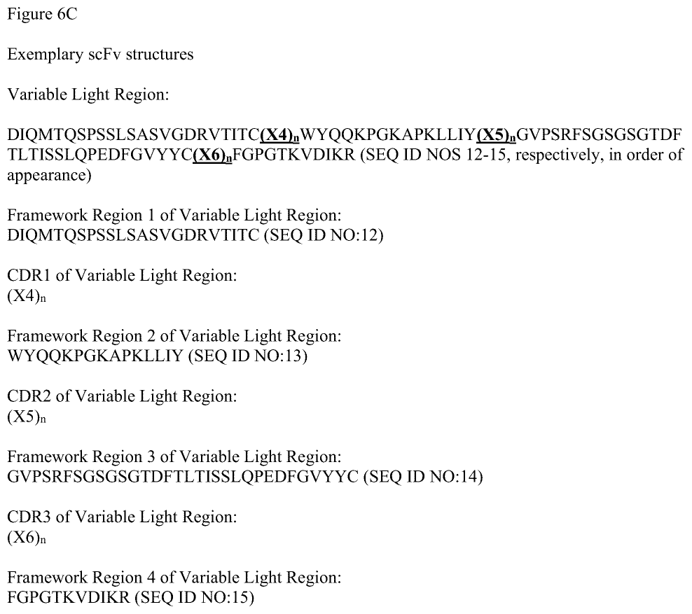

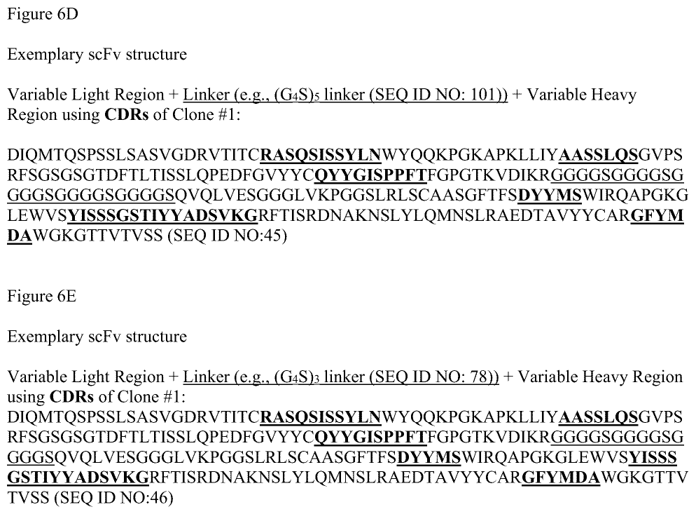

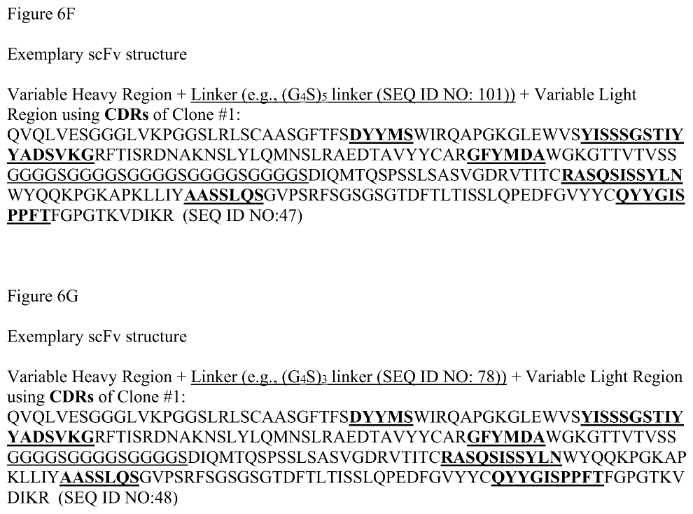

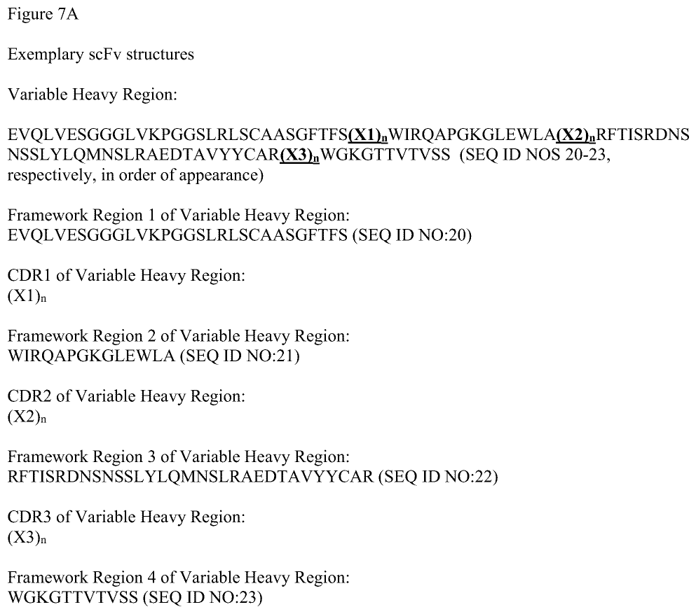

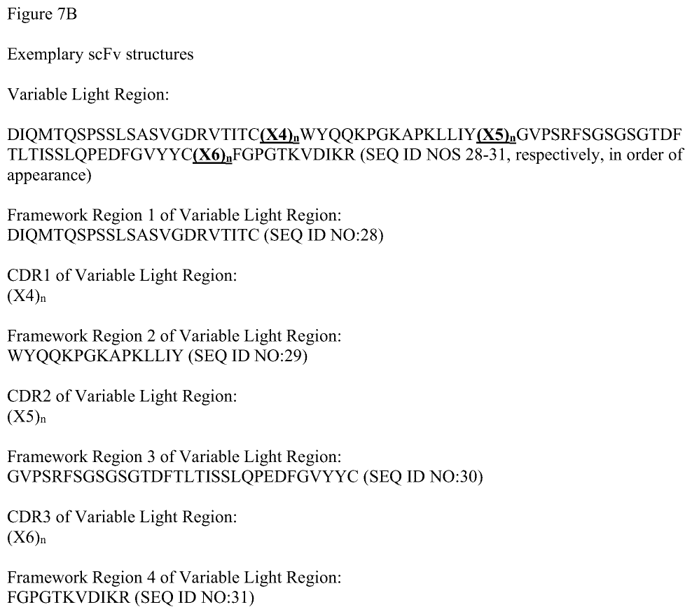

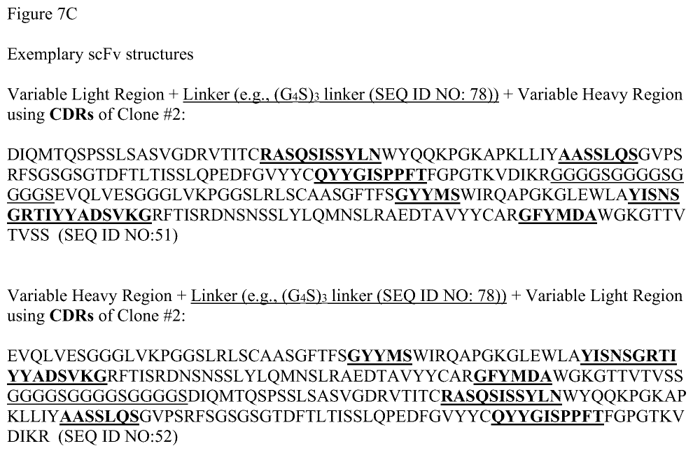

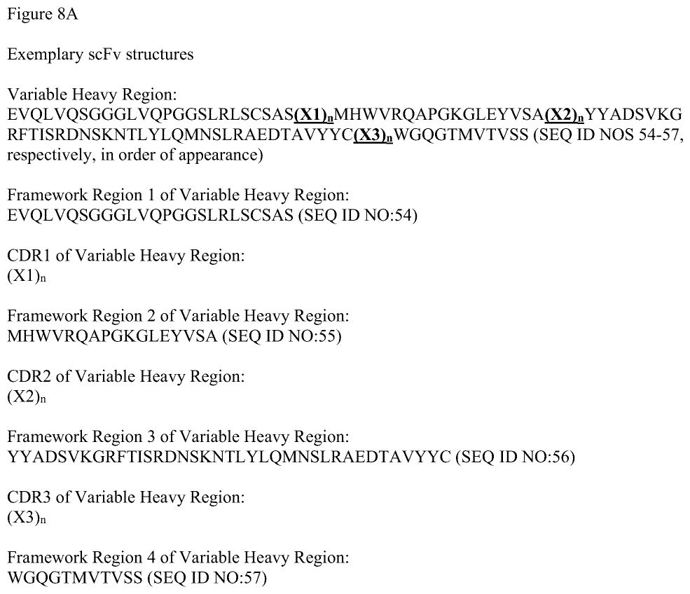

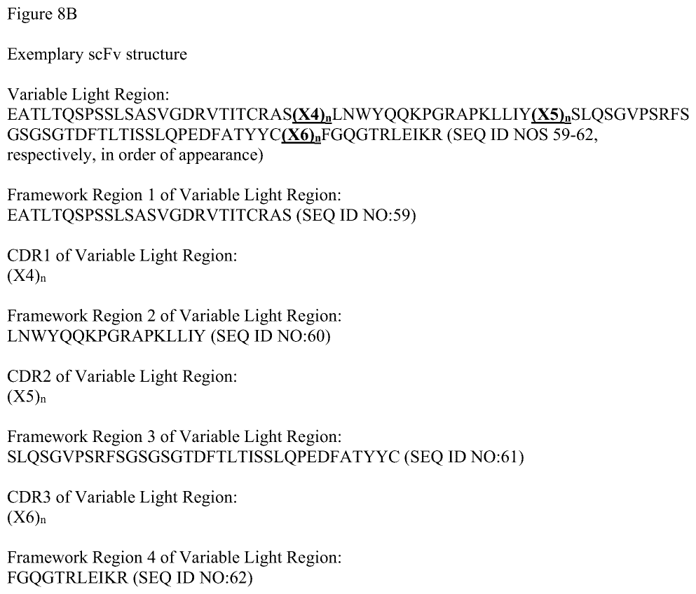

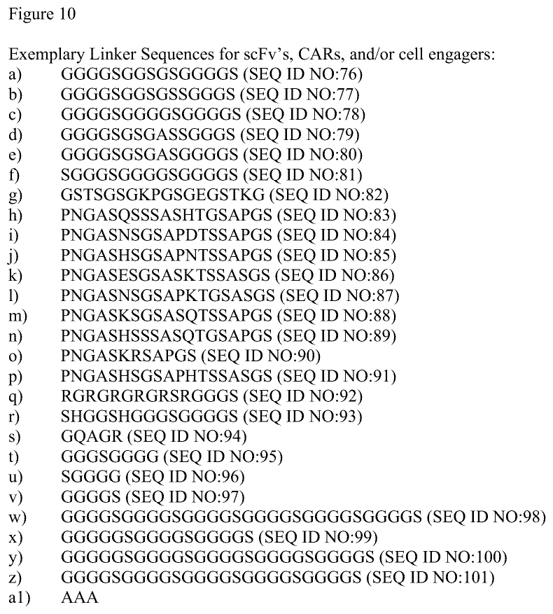

The term “antibody” as used herein includes polyclonal antibodies, monoclonal antibodies, recombinant antibodies, humanized antibodies, human antibodies, chimeric antibodies, multi-specific antibodies (e.g., bispecific antibodies) formed from at least two antibodies, diabodies, single-chain variable fragment antibodies (e.g., scFv antibodies), and tandem single-chain variable fragments antibody (e.g., taFv). A diabody can include two chains, each having a heavy chain variable domain and a light chain variable domain, either from the same or from different antibodies (see, e.g., Hornig and Färber-Schwarz, Methods Mol. Biol., 907:713-27 (2012); and Brinkmann and Kontermann, MAbs., 9(2):182-212 (2017)). The two variable regions can be connected by a polypeptide linker (e.g., a polypeptide linker having five to ten residues in length or a polypeptide linker as set forth in FIG. 10). In some cases, an interdomain disulfide bond can be present in one or both of the heavy chain variable domain and light chain variable domain pairs of the diabody. A scFv is a single-chain polypeptide antibody in which the heavy chain variable domain and the light chain variable domain are directly connected or connected via a polypeptide linker (e.g., a polypeptide linker having eight to 18 residues in length or a polypeptide linker as set forth in FIG. 10). See, also, Chen et al., Adv. Drug Deliv. Rev., 65(10):1357-1369 (2013). A scFv can be designed to have an orientation with the heavy chain variable domain being followed by the light chain variable domain or can be designed to have an orientation with the light chain variable domain being followed by the heavy chain variable domain. In both cases, the optional linker can be located between the two domains. Examples of scFv structures of scFv's provided herein include, without limitation, those structures set forth in FIGS. 6A-6G, 7A-7C, 8A-8B, and 9A-9B.

An antibody provided herein can include the CDRs as described herein (e.g., as described in Table 13) and can be configured to be a human antibody, a humanized antibody, or a chimeric antibody. In some cases, an antibody provided herein can include the CDRs as described herein (e.g., as described in Table 13) and can be a monoclonal antibody. In some cases, an antibody provided herein can include the CDRs as described herein (e.g., as described in Table 13) and can be configured as a scFv antibody.

The term “antigen binding fragment” as used herein refers to a fragment of an antibody (e.g., a fragment of a humanized antibody, a fragment of a human antibody, or a fragment of a chimeric antibody) having the ability to bind to an antigen. Examples of antigen binding fragments include, without limitation, Fab, Fab′, or F(ab′)₂ antigen binding fragments. An antigen binding fragment provided herein can include the CDRs as described herein (e.g., as described in Table 13) and can be configured to be a human antigen binding fragment, a humanized antigen binding fragment, or a chimeric antigen binding fragment. In some cases, an antigen binding fragment provided herein can include the CDRs as described herein (e.g., as described in Table 13) and can be a monoclonal antigen binding fragment. In some cases, an antigen binding fragment provided herein can include the CDRs as described herein (e.g., as described in Table 13) and can be configured as an Fab antibody. In some cases, a Fab antibody can include a partial hinge sequence (e.g., SEQ ID NO:73) for disulfide bonding between heavy and light chains of the Fab.

The term “antibody domain” as used herein refers to a domain of an antibody such as a heavy chain variable domain (VH domain) or a light chain variable domain (VL domain) in the absence of one or more other domains of an antibody. In some cases, an antibody domain can be a single antibody domain (e.g., a VH domain or a VL domain) having the ability to bind to an antigen. An antibody domain provided herein can include the CDRs as described herein (e.g., as described in Table 13) and can be a human antibody domain (e.g., a human VH domain), a humanized antibody domain (e.g., a humanized VH domain), or a chimeric antibody domain (e.g., a chimeric VH domain). In some cases, an antibody domain provided herein can include the CDRs as described herein (e.g., as described in Table 13) and can be a monoclonal antibody domain. In some cases, an antibody domain provided herein can include the CDRs as described herein (e.g., as described in Table 13) and can be engineered as a single VH domain or a single VL domain.

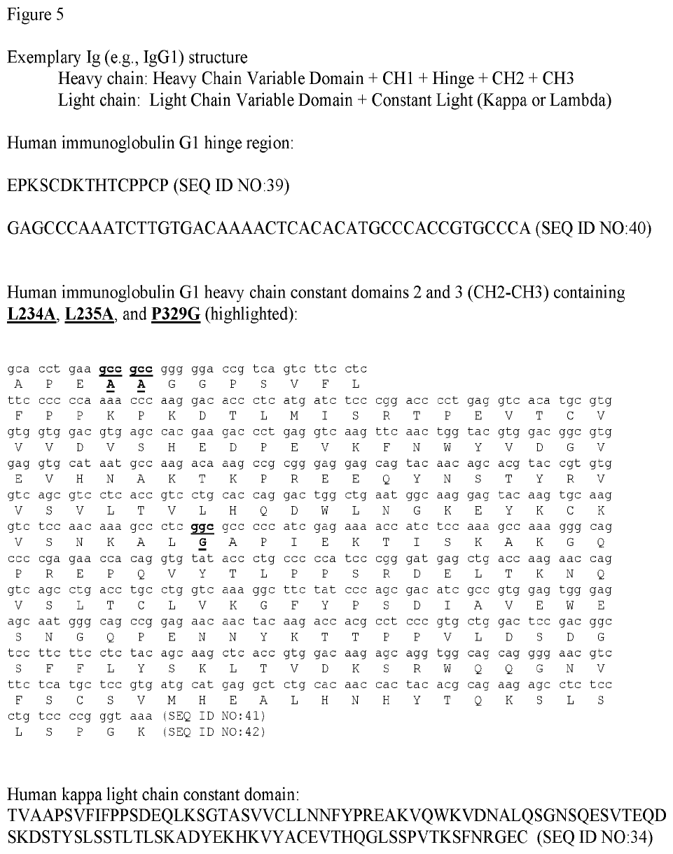

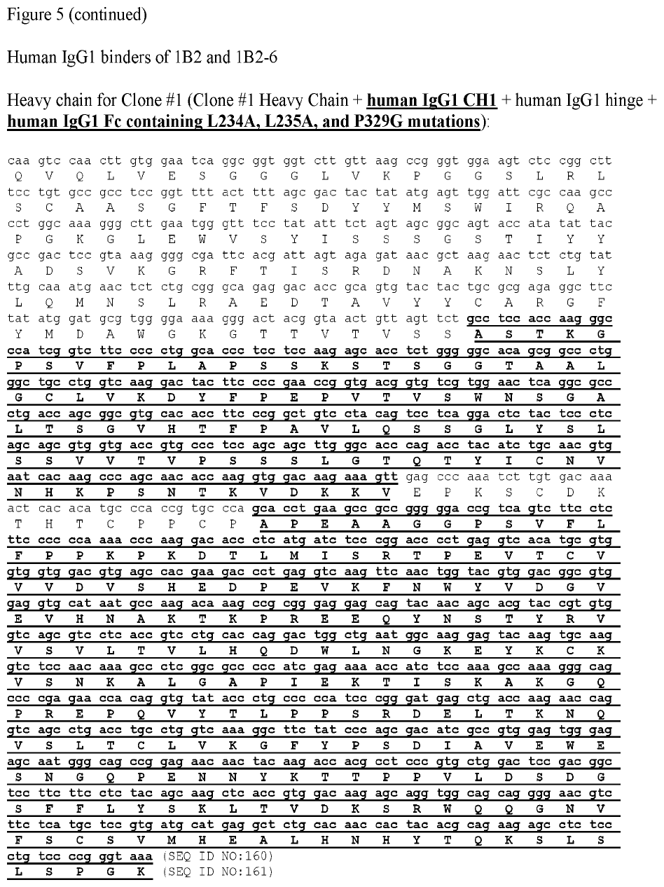

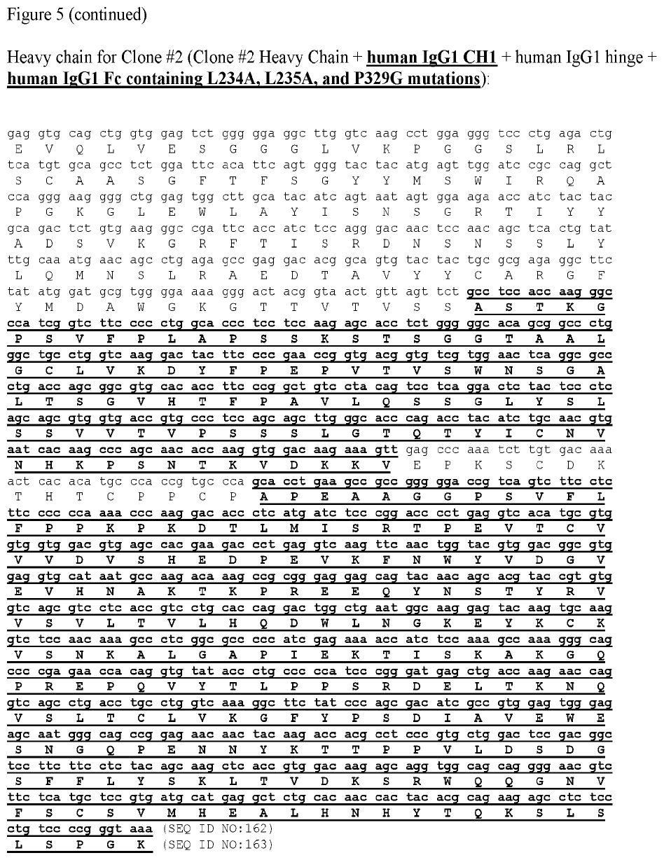

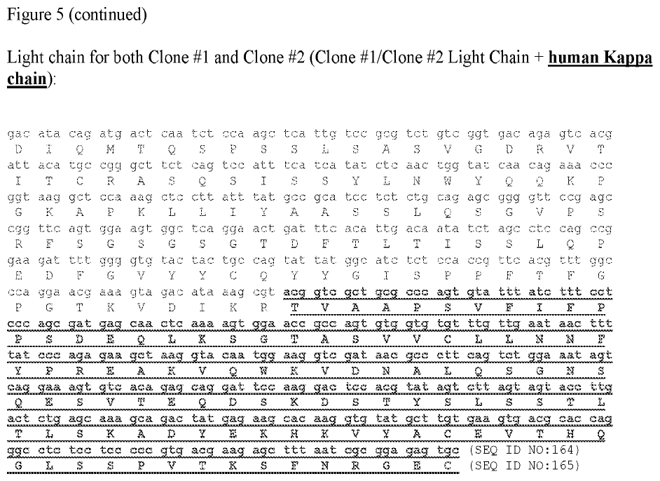

An anti-CD94/NKG2A antibody, anti-CD94/NKG2A antigen binding fragment, or anti-CD94/NKG2A antibody domain provided herein can be of the IgA-, IgD-, IgE-, IgG-, or IgM-type, including IgG- or IgM-types such as, without limitation, IgG₁-, IgG₂-, IgG₃-, IgG₄-, IgM₁-, and IgM₂-types. In some cases, an antibody provided herein (e.g., an anti-CD94/NKG2A antibody) can be a scFv antibody. In some cases, an antigen binding fragment provided herein (e.g., an anti-CD94/NKG2A antibody fragment) can be an Fab. In some cases, an antibody provided herein (e.g., an anti-CD94/NKG2A antibody) can be a fully intact antibody having the structure set forth in FIG. 5. In some cases, an antibody domain provided herein (e.g., an anti-CD94/NKG2A antibody domain) can be a VH domain.

The term “chimeric antigen receptor” as used herein refers to a chimeric polypeptide that is designed to include an optional signal peptide, an antigen binding domain, an optional hinge, a transmembrane domain, and one or more intracellular signaling domains. As described herein, the antigen binding domain of a CAR provided herein can be designed to bind to a CD94/NKG2A polypeptide (e.g., a human CD94/NKG2A polypeptide). For example, a CAR provided herein can be designed to include the components of an antibody, antigen binding fragment, and/or antibody domain described herein (e.g., a combination of CDRs) as an antigen binding domain provided that that antigen binding domain has the ability to bind to a CD94/NKG2A polypeptide (e.g., a human CD94/NKG2A polypeptide). In some examples, a CAR provided herein can be designed to include an antigen binding domain that includes two sets of three CDRs (e.g., CDR1, CDR2, and CDR3 of a heavy chain and CDR1, CDR2, and CDR3 of a light chain) of an antigen binding fragment provided herein (e.g., SEQ ID NOs:1-3 and 9-11 or SEQ ID NOs:17-19 and 25-27). In some cases, an antigen binding domain of a CAR targeting a CD94/NKG2A polypeptide can be designed to include a VH domain described herein or a scFv antibody described herein.

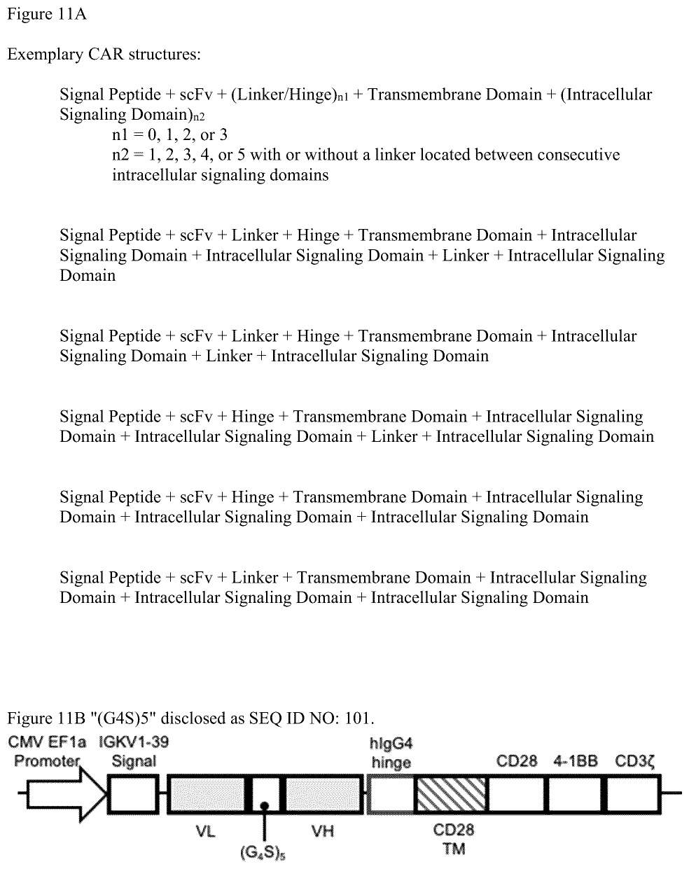

Examples of CAR structures that can be used to make a CAR provided herein include, without limitation, those set forth in FIGS. 11A and 11B.

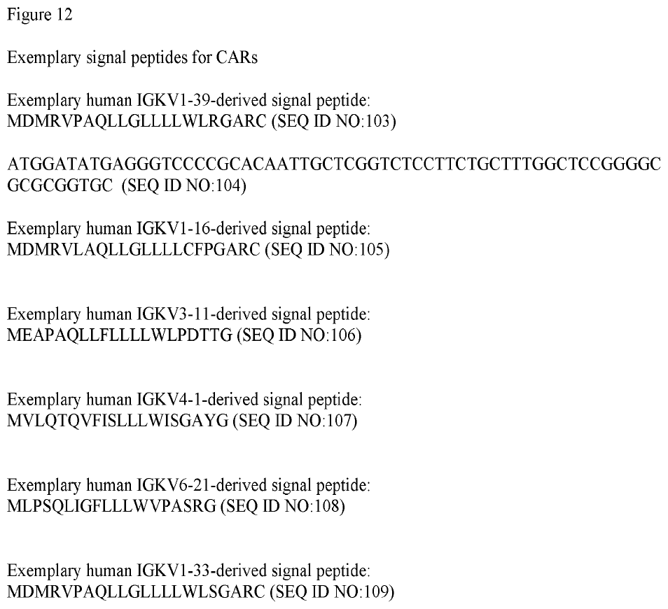

In some cases, a CAR provided herein can be designed to include a signal peptide. Any appropriate signal peptide can be used to design a CAR described herein. Examples of signal peptide that can be used to make a CAR described herein include without limitation, a human IGKV1-39-derived signal peptide, IGKV1-16, IGKV1-33, IGKV3-11, IGKV4-1, or IGKV6-21. In some cases, a CAR provided herein can be designed to include a signal peptide that comprises, consists essentially of, or consists of one of the amino acid sequences set forth in FIG. 12. In some cases, a CAR provided herein can be designed to include a signal peptide that comprises, consists essentially of, or consists of one of the amino acid sequences set forth in FIG. 12 with one, two, three, four, five, six, seven, eight, nine, or ten amino acid deletions, additions, substitutions, or combinations thereof. In some cases, a CAR provided herein can be designed to include a signal peptide that comprises, consists essentially of, or consists of one of the amino acid sequences set forth in FIG. 12 with two or less, three or less, four or less, five or less, six or less, seven or less, eight or less, nine or less, or ten or less amino acid deletions, additions, substitutions, or combinations thereof.

In some cases, a CAR provided herein can be designed to include a hinge. Any appropriate hinge can be used to design a CAR described herein. Examples of hinges that can be used to make a CAR described herein include, without limitation, Ig-derived hinges (e.g., an IgG1-derived hinge, an IgG2-derived hinge, or an IgG4-derived hinge), Ig-derived hinges containing a CD2 domain and a CD3 domain, Ig-derived hinges containing a CD2 domain and lacking a CD3 domain, Ig-derived hinges containing a CD3 domain and lacking a CD2 domain, Ig-derived hinges lacking a CD2 domain and lacking a CD3 domain, CD8α-derived hinges, CD28-derived hinges, and CD3ζ (-derived hinges. A CAR provided herein can be designed to include a hinge of any appropriate length. For example, a CAR provided herein can be designed to include a hinge that is from about 3 to about 75 (e.g., from about 3 to about 65, from about 3 to about 50, from about 5 to about 75, from about 10 to about 75, from about 5 to about 50, from about 10 to about 50, from about 10 to about 40, or from about 10 to about 30) amino acid residues in length. In some cases, a linker sequence can be used as a hinge to make a CAR described herein. For example, any one of the linker sequences set forth in FIG. 10 can be used as a hinge of a CAR described herein.

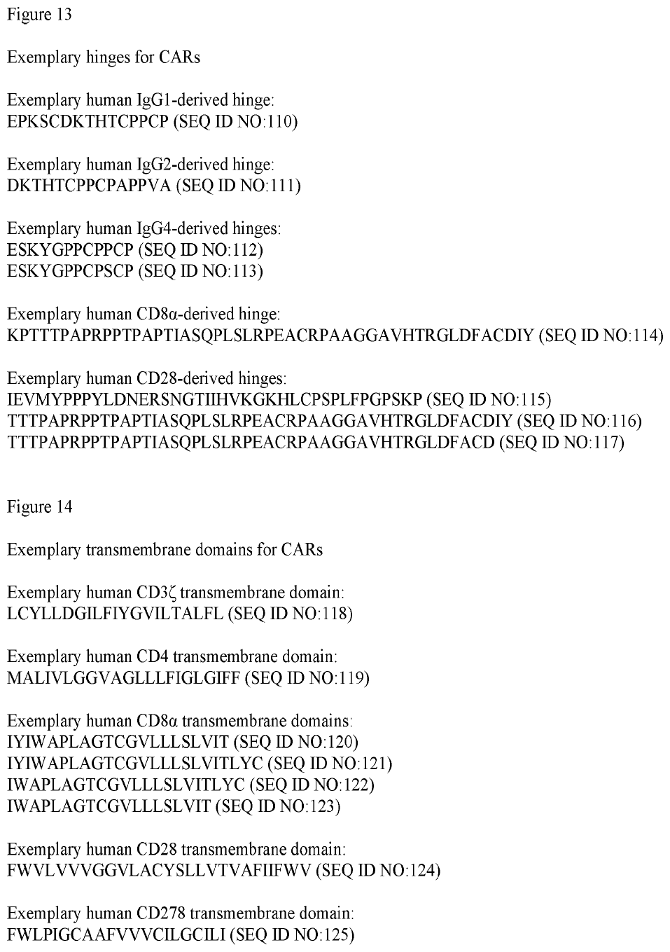

In some cases, a CAR provided herein can be designed to include a hinge that comprises, consists essentially of, or consists of one of the amino acid sequences set forth in FIG. 10 or FIG. 13. In some cases, a CAR provided herein can be designed to include a hinge that comprises, consists essentially of, or consists of one of the amino acid sequences set forth in FIG. 10 or FIG. 13 with one, two, three, four, five, six, seven, eight, nine, or ten amino acid deletions, additions, substitutions, or combinations thereof. In some cases, a CAR provided herein can be designed to include a hinge that comprises, consists essentially of, or consists of one of the amino acid sequences set forth in FIG. 10 or FIG. 13 with two or less, three or less, four or less, five or less, six or less, seven or less, eight or less, nine or less, or ten or less amino acid deletions, additions, substitutions, or combinations thereof.

A CAR provided herein can be designed to include any appropriate transmembrane domain. For example, the transmembrane domain of a CAR provided herein can be, without limitation, a CD3ζ (transmembrane domain, a CD4 transmembrane domain, a CD8α transmembrane domain, a CD28 transmembrane domain, and a 4-1BB transmembrane domain. In some cases, a CAR provided herein can be designed to include a transmembrane domain that comprises, consists essentially of, or consists of one of the amino acid sequences set forth in FIG. 14. In some cases, a CAR provided herein can be designed to include a transmembrane domain that comprises, consists essentially of, or consists of one of the amino acid sequences set forth in FIG. 14 with one, two, three, four, five, six, seven, eight, nine, or ten amino acid deletions, additions, substitutions, or combinations thereof. In some cases, a CAR provided herein can be designed to include a transmembrane domain that comprises, consists essentially of, or consists of one of the amino acid sequences set forth in FIG. 14 with two or less, three or less, four or less, five or less, six or less, seven or less, eight or less, nine or less, or ten or less amino acid deletions, additions, substitutions, or combinations thereof.

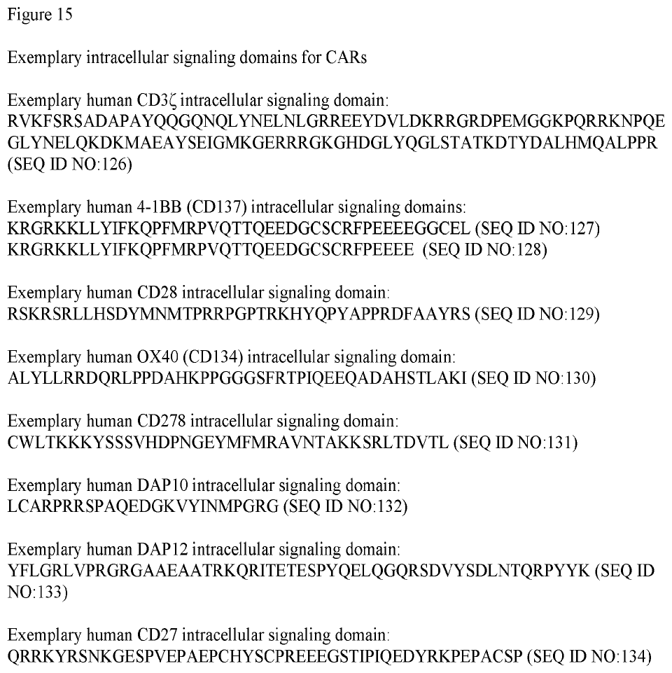

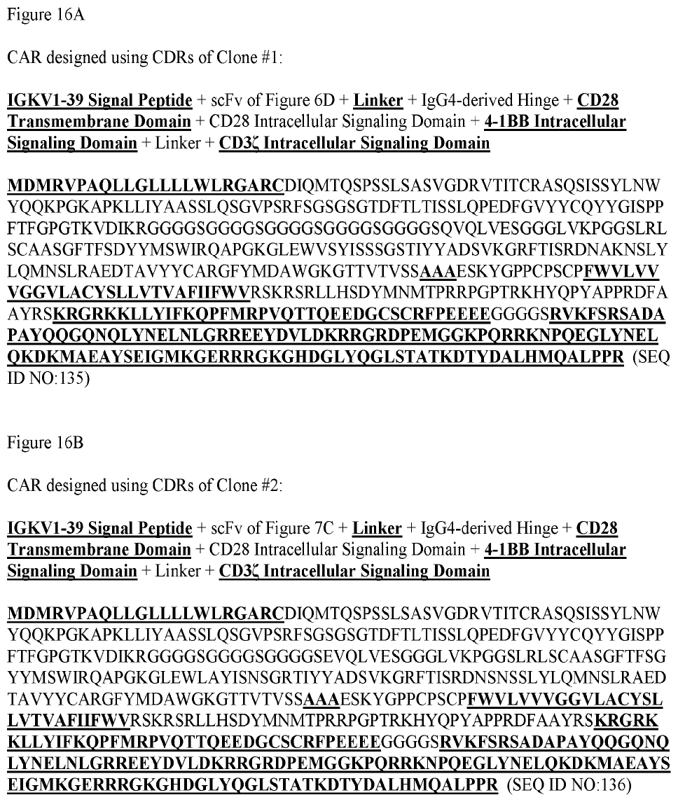

A CAR provided herein can be designed to include one or more intracellular signaling domains. For example, a CAR provided herein can be designed to include one, two, three, or four intracellular signaling domains. Any appropriate intracellular signaling domain or combination of intracellular signaling domains can be used to make a CAR described herein. Examples of intracellular signaling domains that can be used to make a CAR described herein include, without limitation, CD3ζ (intracellular signaling domains, CD27 intracellular signaling domains, CD28 intracellular signaling domains, OX40 (CD134) intracellular signaling domains, 4-1BB (CD137) intracellular signaling domains, CD278 intracellular signaling domains, DAP10 intracellular signaling domains, and DAP12 intracellular signaling domains. In some cases, a CAR described herein can be designed to be a first generation CAR having a CD3ζ (intracellular signaling domain. In some cases, a CAR described herein can be designed to be a second generation CAR having a CD28 intracellular signaling domain followed by a CD3ζ (intracellular signaling domain. In some cases, a CAR described herein can be designed to be a third generation CAR having (a) a CD28 intracellular signaling domain followed by (b) a CD27 intracellular signaling domain, an OX40 intracellular signaling domains, or a 4-1BB intracellular signaling domain followed by (c) a CD3ζ (intracellular signaling domain. In some cases, a CAR provided herein can be designed to include at least one intracellular signaling domain that comprises, consists essentially of, or consists of one of the amino acid sequences set forth in FIG. 15. In some cases, a CAR provided herein can be designed to include at least one intracellular signaling domain that comprises, consists essentially of, or consists of one of the amino acid sequences set forth in FIG. 15 with one, two, three, four, five, six, seven, eight, nine, or ten amino acid deletions, additions, substitutions, or combinations thereof, provided that that intracellular signaling domain has at least some activity to activate intracellular signaling. In some cases, a CAR provided herein can be designed to include at least one intracellular signaling domain that comprises, consists essentially of, or consists of one of the amino acid sequences set forth in FIG. 15 with two or less, three or less, four or less, five or less, six or less, seven or less, eight or less, nine or less, or ten or less amino acid deletions, additions, substitutions, or combinations thereof, provided that that intracellular signaling domain has at least some activity to activate intracellular signaling.

In some cases, a CAR targeting a CD94/NKG2A polypeptide can be designed to include an scFv having a heavy chain variable domain comprising SEQ ID NO:1, SEQ ID NO:2, and SEQ ID NO:3, followed by a linker such as a linker set forth in FIG. 10, followed by a light chain variable domain comprising SEQ ID NO:9, SEQ ID NO:10, and SEQ ID NO:11, followed by a hinge such as a hinge/linker set forth in FIG. 10 or FIG. 13 (e.g., an IgG4-derived hinge, a CD8α hinge, or a linker plus IgG4-derived hinge), followed by a transmembrane domain such as a transmembrane domain set forth in FIG. 14 (e.g., a human CD28 transmembrane domain or a CD8α transmembrane domain), followed by one or more intracellular signaling domains such as one or more intracellular signaling domain set forth in FIG. 15 (e.g., a human 4-1BB intracellular signaling domain followed by a human CD3ζ (intracellular signaling domain). For example, a CAR targeting a CD94/NKG2A polypeptide can be designed to include an scFv having a heavy chain variable domain comprising SEQ ID NO:1, SEQ ID NO:2, and SEQ ID NO:3, followed by SEQ ID NO:100, followed by a light chain variable domain comprising SEQ ID NO:9, SEQ ID NO:10, and SEQ ID NO:11, followed by the sequence “AAA”, followed by SEQ ID NO:113, followed by SEQ ID NO:124, followed by SEQ ID NO:129, followed by SEQ ID NO:128, followed by SEQ ID NO:97, followed by SEQ ID NO:126.

In some cases, a CAR targeting a CD94/NKG2A polypeptide can be designed to include an scFv having a heavy chain variable domain comprising SEQ ID NO:8, followed by a linker such as a linker set forth in FIG. 10, followed by a light chain variable domain comprising SEQ ID NO:16, followed by a hinge such as a hinge/linker set forth in FIG. 10 or FIG. 13 (e.g., an IgG4-derived hinge, a CD8α hinge, or a linker plus IgG4-derived hinge), followed by a transmembrane domain such as a transmembrane domain set forth in FIG. 14 (e.g., a human CD28 transmembrane domain or a CD8α transmembrane domain), followed by one or more intracellular signaling domains such as one or more intracellular signaling domain set forth in FIG. 15 (e.g., a human 4-1BB intracellular signaling domain followed by a human CD3ζ (intracellular signaling domain). For example, a CAR targeting a CD94/NKG2A polypeptide can be designed to include an scFv having a heavy chain variable domain comprising SEQ ID NO:8, followed by SEQ ID NO: 100, followed by a light chain variable domain comprising SEQ ID NO:16, followed by the sequence “AAA”, followed by SEQ ID NO:113, followed by SEQ ID NO:124, followed by SEQ ID NO:129, followed by SEQ ID NO:128, followed by SEQ ID NO:97, followed by SEQ ID NO:126.

In some cases, a CAR targeting a CD94/NKG2A polypeptide can be designed to include an scFv having a light chain variable domain comprising SEQ ID NO:9, SEQ ID NO:10, and SEQ ID NO:11, followed by a linker such as a linker set forth in FIG. 10, followed by a heavy chain variable domain comprising SEQ ID NO:1, SEQ ID NO:2, and SEQ ID NO:3, followed by a hinge such as a hinge/linker set forth in FIG. 10 or FIG. 13 (e.g., an IgG4-derived hinge, a CD8α hinge, or a linker plus IgG4-derived hinge), followed by a transmembrane domain such as a transmembrane domain set forth in FIG. 14 (e.g., a human CD28 transmembrane domain or a CD8α transmembrane domain), followed by one or more intracellular signaling domains such as one or more intracellular signaling domain set forth in FIG. 15 (e.g., a human 4-1BB intracellular signaling domain followed by a human CD3ζ (intracellular signaling domain). For example, a CAR targeting a CD94/NKG2A polypeptide can be designed to include an scFv having a light chain variable domain comprising SEQ ID NO:9, SEQ ID NO:10, and SEQ ID NO:11, followed by SEQ ID NO:100, followed by a heavy chain variable domain comprising SEQ ID NO:1, SEQ ID NO:2, and SEQ ID NO:3, followed by the sequence “AAA”, followed by SEQ ID NO:113, followed by SEQ ID NO:124, followed by SEQ ID NO:129, followed by SEQ ID NO:128, followed by SEQ ID NO:97, followed by SEQ ID NO:126 (see, e.g., FIG. 16).

In some cases, a CAR targeting a CD94/NKG2A polypeptide can be designed to include an scFv having a light chain variable domain comprising SEQ ID NO:16, followed by a linker such as a linker set forth in FIG. 10, followed by a heavy chain variable domain comprising SEQ ID NO:8, followed by a hinge such as a hinge/linker set forth in FIG. 10 or FIG. 13 (e.g., an IgG4-derived hinge, a CD8α hinge, or a linker plus IgG4-derived hinge), followed by a transmembrane domain such as a transmembrane domain set forth in FIG. 14 (e.g., a human CD28 transmembrane domain or a CD8α transmembrane domain), followed by one or more intracellular signaling domains such as one or more intracellular signaling domain set forth in FIG. 15 (e.g., a human 4-1BB intracellular signaling domain followed by a human CD3ζ (intracellular signaling domain). For example, a CAR targeting a CD94/NKG2A polypeptide can be designed to include an scFv having a light chain variable domain comprising SEQ ID NO:16, followed by SEQ ID NO:100, followed by a heavy chain variable domain comprising SEQ ID NO:8, followed by the sequence “AAA”, followed by SEQ ID NO:113, followed by SEQ ID NO:124, followed by SEQ ID NO:129, followed by SEQ ID NO:128, followed by SEQ ID NO:97, followed by SEQ ID NO:126 (see, e.g., FIG. 16).

In some cases, a CAR targeting a CD94/NKG2A polypeptide can be designed to include an scFv having a heavy chain variable domain comprising SEQ ID NO:17, SEQ ID NO:18, and SEQ ID NO:19, followed by a linker such as a linker set forth in FIG. 10, followed by a light chain variable domain comprising SEQ ID NO:25, SEQ ID NO:26, and SEQ ID NO:27, followed by a hinge such as a hinge/linker set forth in FIG. 10 or FIG. 13 (e.g., an IgG4-derived hinge, a CD8α hinge, or a linker plus IgG4-derived hinge), followed by a transmembrane domain such as a transmembrane domain set forth in FIG. 14 (e.g., a human CD28 transmembrane domain or a CD8α transmembrane domain), followed by one or more intracellular signaling domains such as one or more intracellular signaling domain set forth in FIG. 15 (e.g., a human 4-1BB intracellular signaling domain followed by a human CD3ζ (intracellular signaling domain). For example, a CAR targeting a CD94/NKG2A polypeptide can be designed to include an scFv having a heavy chain variable domain comprising SEQ ID NO:17, SEQ ID NO:18, and SEQ ID NO:19, followed by SEQ ID NO:100, followed by a light chain variable domain comprising SEQ ID NO:25, SEQ ID NO:26, and SEQ ID NO:27, followed by the sequence “AAA”, followed by SEQ ID NO:113, followed by SEQ ID NO:124, followed by SEQ ID NO:129, followed by SEQ ID NO:128, followed by SEQ ID NO:97, followed by SEQ ID NO:126.

In some cases, a CAR targeting a CD94/NKG2A polypeptide can be designed to include an scFv having a heavy chain variable domain comprising SEQ ID NO:24, followed by a linker such as a linker set forth in FIG. 10, followed by a light chain variable domain comprising SEQ ID NO:32, followed by a hinge such as a hinge/linker set forth in FIG. 10 or FIG. 13 (e.g., an IgG4-derived hinge, a CD8α hinge, or a linker plus IgG4-derived hinge), followed by a transmembrane domain such as a transmembrane domain set forth in FIG. 14 (e.g., a human CD28 transmembrane domain or a CD8α transmembrane domain), followed by one or more intracellular signaling domains such as one or more intracellular signaling domain set forth in FIG. 15 (e.g., a human 4-1BB intracellular signaling domain followed by a human CD3ζ (intracellular signaling domain). For example, a CAR targeting a CD94/NKG2A polypeptide can be designed to include an scFv having a heavy chain variable domain comprising SEQ ID NO:24, followed by SEQ ID NO:100, followed by a light chain variable domain comprising SEQ ID NO:32, followed by the sequence “AAA”, followed by SEQ ID NO:113, followed by SEQ ID NO:124, followed by SEQ ID NO:129, followed by SEQ ID NO:128, followed by SEQ ID NO:97, followed by SEQ ID NO:126.

In some cases, a CAR targeting a CD94/NKG2A polypeptide can be designed to include an scFv having a light chain variable domain comprising SEQ ID NO:25, SEQ ID NO:26, and SEQ ID NO:27, followed by a linker such as a linker set forth in FIG. 10, followed by a heavy chain variable domain comprising SEQ ID NO:17, SEQ ID NO:18, and SEQ ID NO:19, followed by a hinge such as a hinge/linker set forth in FIG. 10 or FIG. 13 (e.g., an IgG4-derived hinge, a CD8α hinge, or a linker plus IgG4-derived hinge), followed by a transmembrane domain such as a transmembrane domain set forth in FIG. 14 (e.g., a human CD28 transmembrane domain or a CD8α transmembrane domain), followed by one or more intracellular signaling domains such as one or more intracellular signaling domain set forth in FIG. 15 (e.g., a human 4-1BB intracellular signaling domain followed by a human CD3ζ (intracellular signaling domain). For example, a CAR targeting a CD94/NKG2A polypeptide can be designed to include an scFv having a light chain variable domain comprising SEQ ID NO:25, SEQ ID NO:26, and SEQ ID NO:27, followed by SEQ ID NO:100, followed by a heavy chain variable domain comprising SEQ ID NO:17, SEQ ID NO:18, and SEQ ID NO:19, followed by the sequence “AAA”, followed by SEQ ID NO:113, followed by SEQ ID NO:124, followed by SEQ ID NO:129, followed by SEQ ID NO:128, followed by SEQ ID NO:97, followed by SEQ ID NO:126 (see, e.g., FIG. 16B).

In some cases, a CAR targeting a CD94/NKG2A polypeptide can be designed to include an scFv having a light chain variable domain comprising SEQ ID NO:32, followed by a linker such as a linker set forth in FIG. 10, followed by a heavy chain variable domain comprising SEQ ID NO:24, followed by a hinge such as a hinge/linker set forth in FIG. 10 or FIG. 13 (e.g., an IgG4-derived hinge, a CD8α hinge, or a linker plus IgG4-derived hinge), followed by a transmembrane domain such as a transmembrane domain set forth in FIG. 14 (e.g., a human CD28 transmembrane domain or a CD8α transmembrane domain), followed by one or more intracellular signaling domains such as one or more intracellular signaling domain set forth in FIG. 15 (e.g., a human 4-1BB intracellular signaling domain followed by a human CD3ζ ( intracellular signaling domain). For example, a CAR targeting a CD94/NKG2A polypeptide can be designed to include an scFv having a light chain variable domain comprising SEQ ID NO:32, followed by SEQ ID NO:100, followed by a heavy chain variable domain comprising SEQ ID NO:24, followed by the sequence “AAA”, followed by SEQ ID NO:113, followed by SEQ ID NO:124, followed by SEQ ID NO:129, followed by SEQ ID NO:128, followed by SEQ ID NO:97, followed by SEQ ID NO:126 (see, e.g., FIG. 16B).

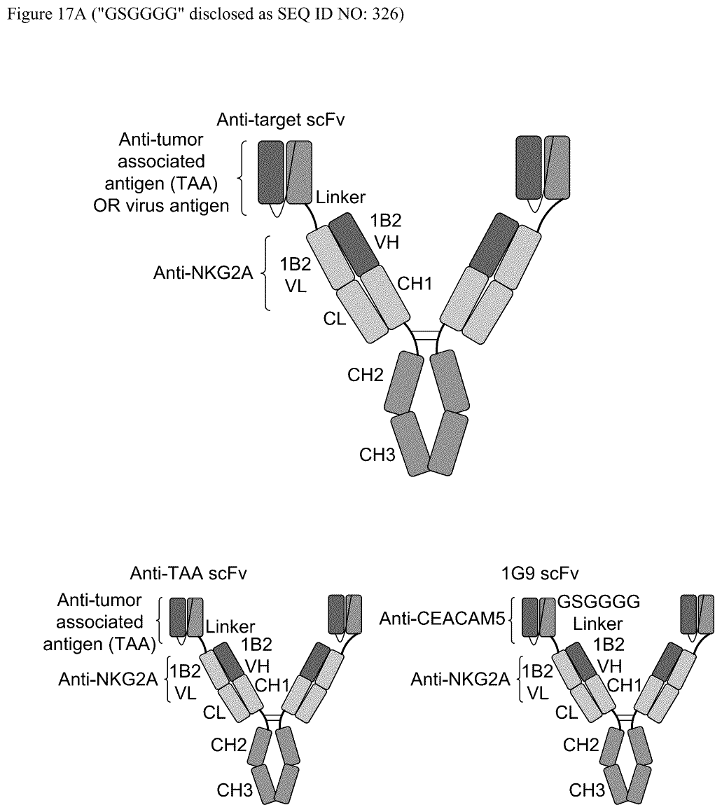

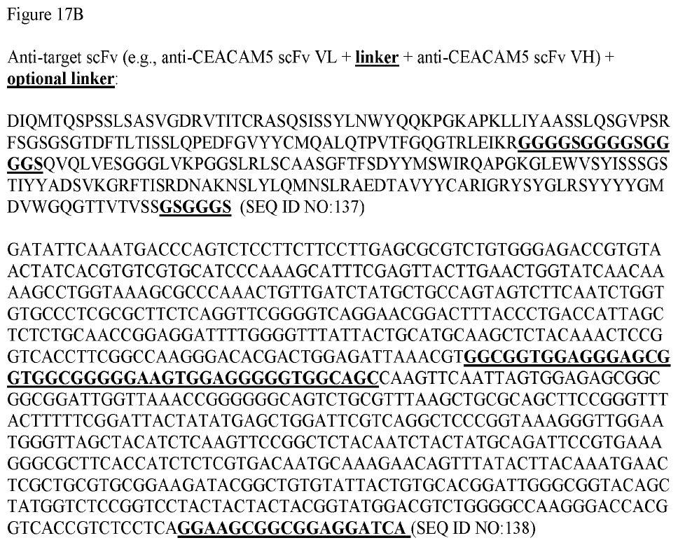

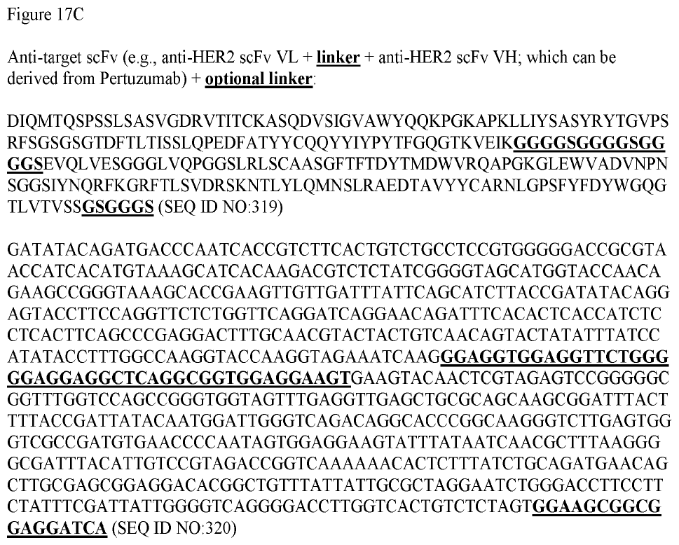

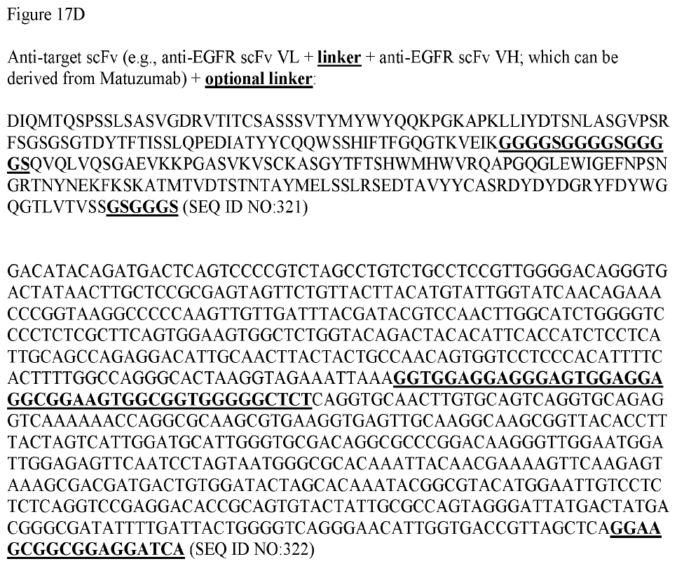

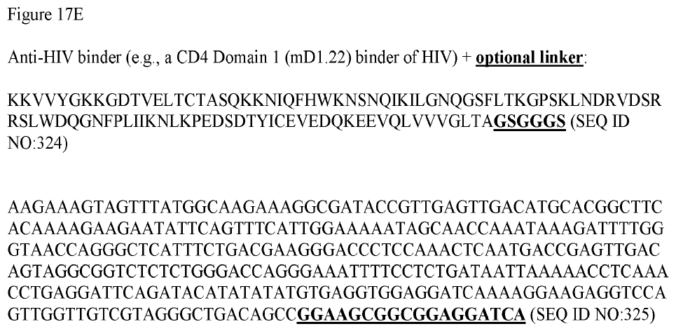

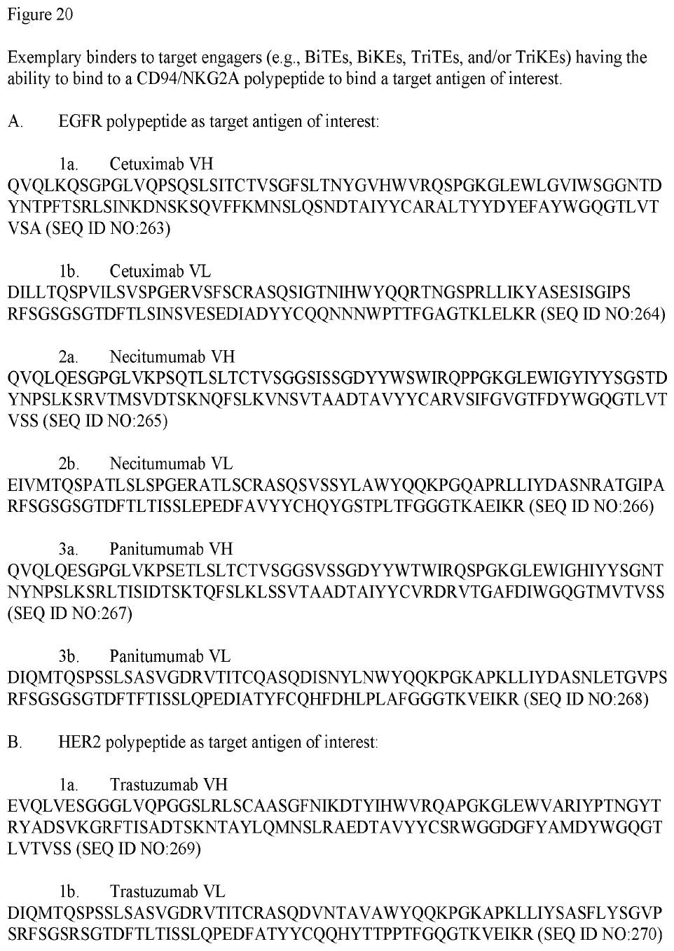

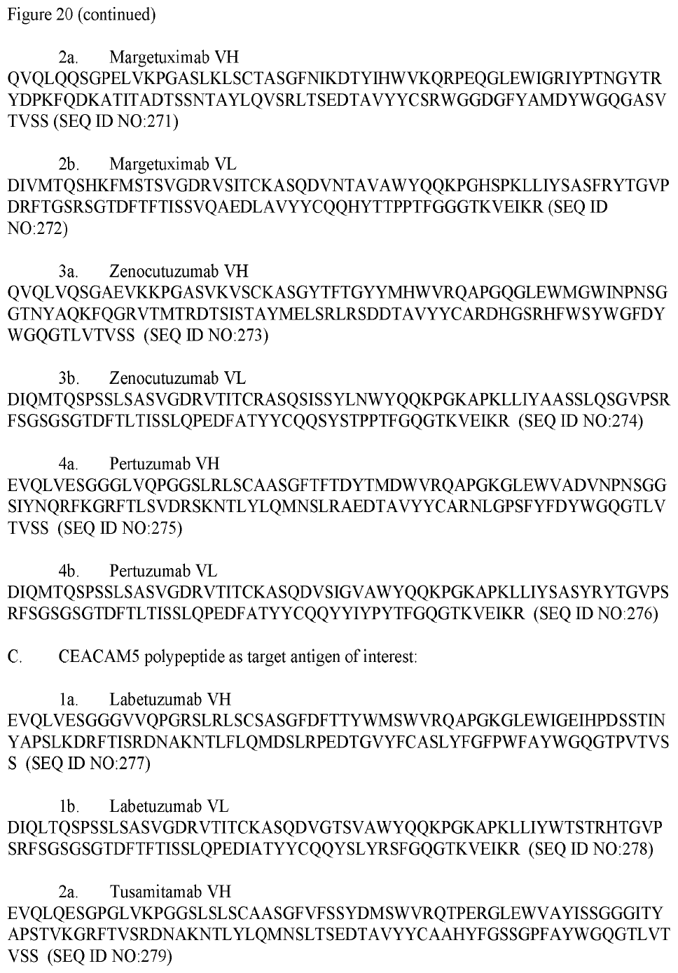

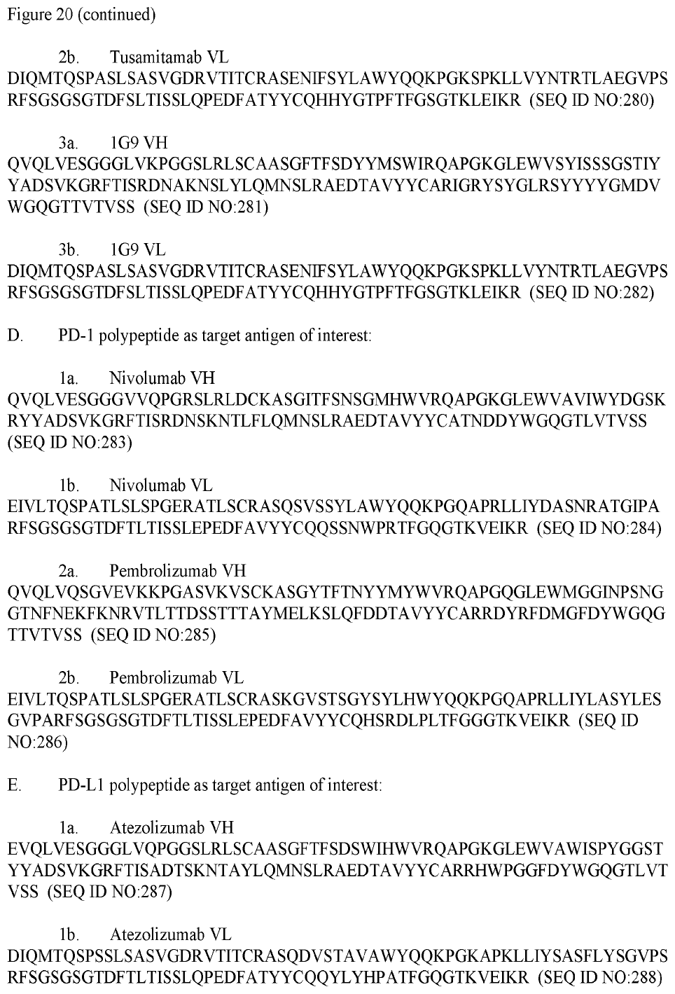

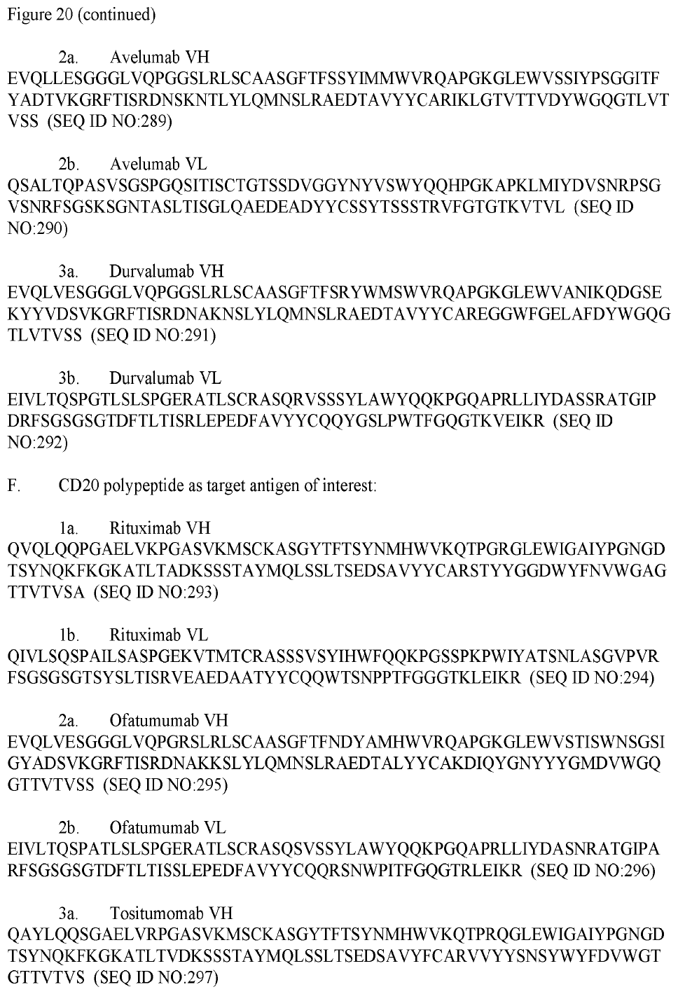

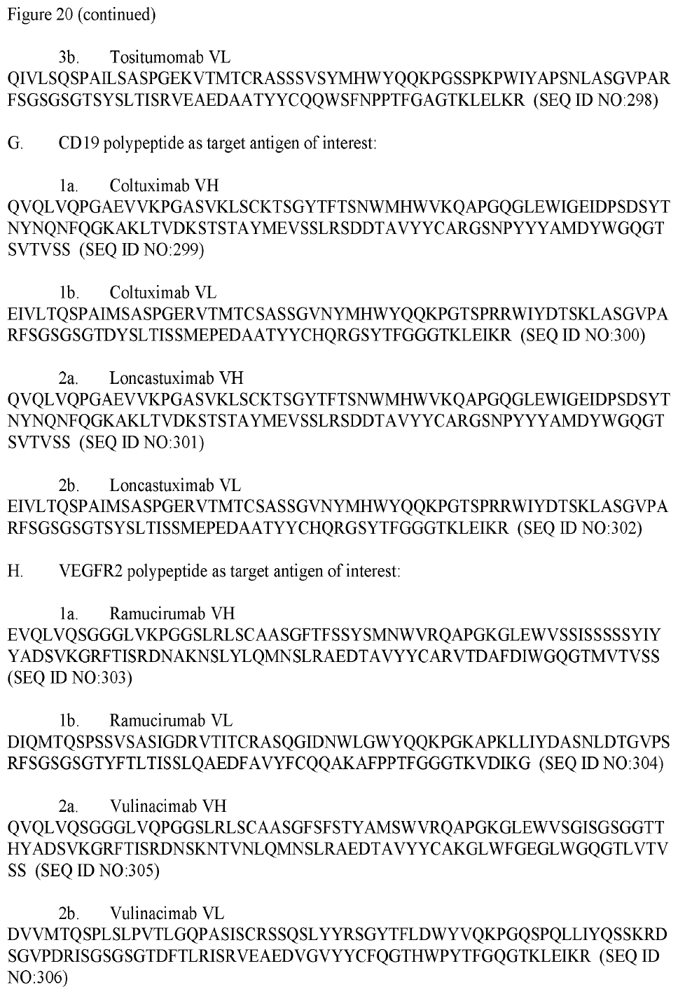

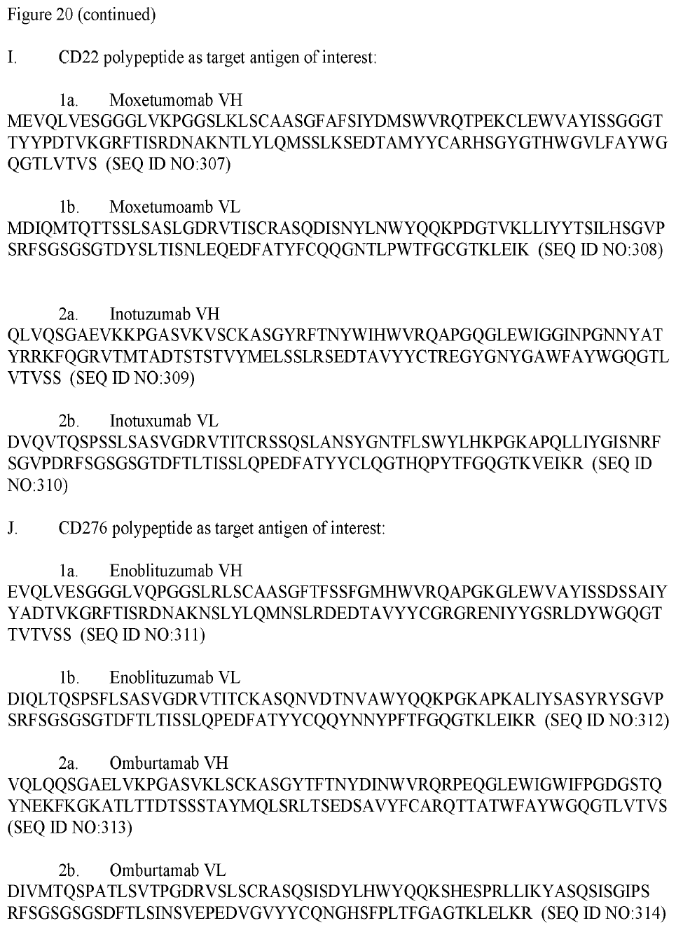

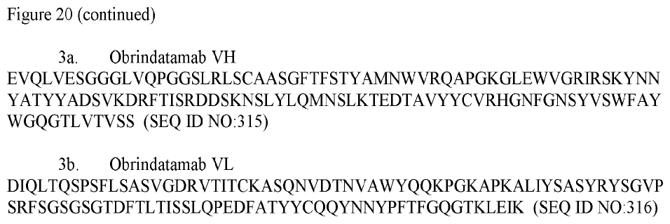

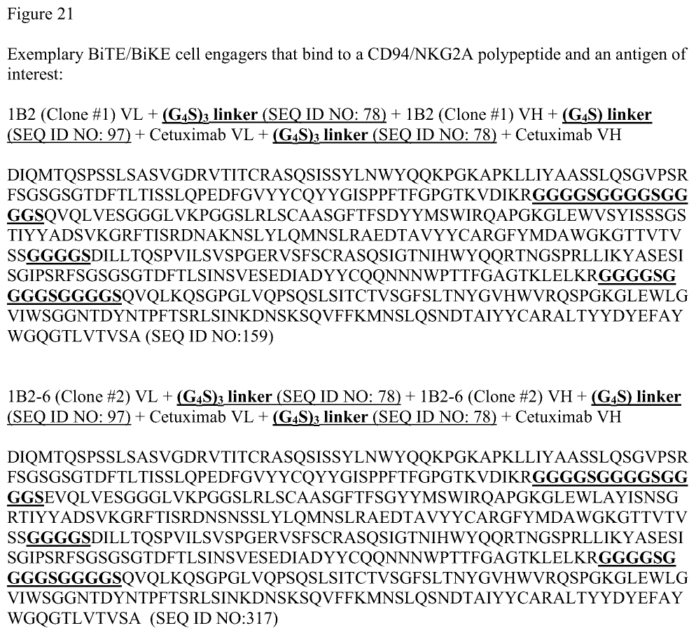

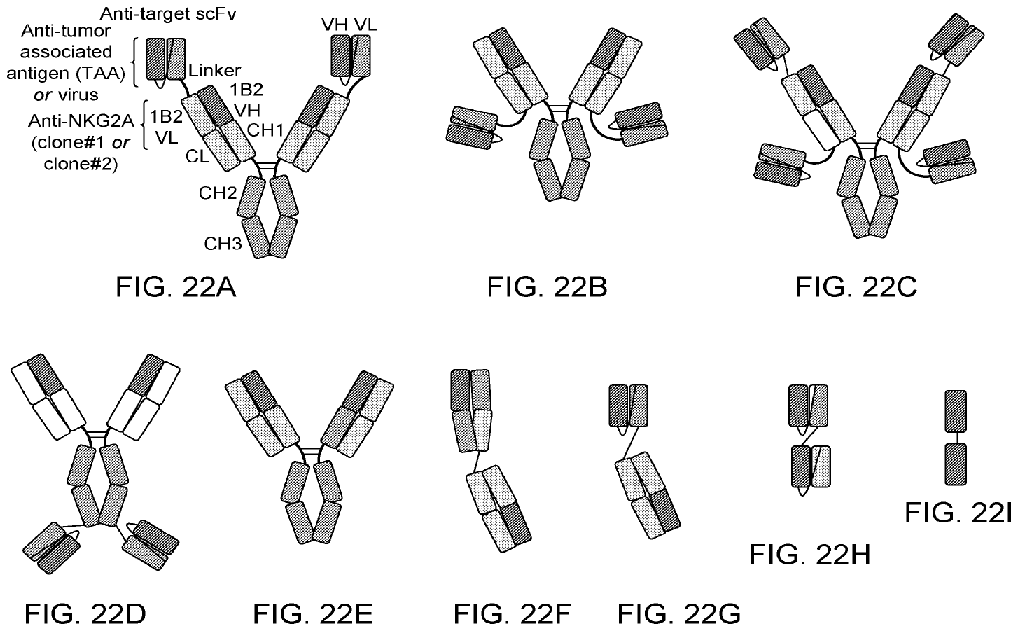

The term “cell engager” as used herein refers to a polypeptide that includes two or more antigen binding domains (e.g., two, three, or four antigen binding domains) and has the ability to link two cells together. Examples of cell engagers include, without limitation, BiTEs, TriTEs, BiKEs, and TriKEs. In general, a cell engager provided herein can be designed to include at least one antigen binding domain having the ability to bind to a CD94/NKG2A polypeptide (e.g., a human CD94/NKG2A polypeptide) expressed on the surface of a cell (e.g., a CD94/NKG2A⁺ CD8⁺ T cell and/or a CD94/NKG2A⁺ NK cell) and at least one antigen binding domain having the ability to bind to a target antigen of interest. In some cases, a cell engager described herein can link a CD94/NKG2A⁺ cell (e.g., a CD94/NKG2A⁺ CD8⁺ T cell and/or a CD94/NKG2A⁺ NK cell) to another cell (e.g., a target cell expressing a target antigen of interest) via the two or more antigen binding domains of the cell engager. Examples of cell engager structures of cell engagers provided herein include, without limitation, the structures set forth in FIG. 17A and/or FIG. 22. In some cases, the anti-CEACAM5 scFv depicted in FIG. 17B can be replaced with a different antigen binding domain having the ability to bind to a target antigen of interest expressed on the surface of a cell (e.g., a cancer cell and/or a virally infected cell) such as those shown in FIG. 17C or FIG. 17D. Examples of antigen binding domains having the ability to bind to a target antigen of interest expressed on the surface of a cell (e.g., a cancer cell and/or a virally infected cell) include, without limitation, antibodies, antigen binding fragments, and antibody domains having the ability to bind to an EGFR polypeptide (see, e.g., FIG. 17D), an IGF-1R polypeptide, an HER2 polypeptide (see, e.g., FIG. 17C), an HER3 polypeptide, an EphA4 polypeptide, a PD-1 polypeptide, a PD-L1 polypeptide, a CTLA4 polypeptide, a DLL4 polypeptide, an ADAM10 polypeptide, a c-Met polypeptide, a GPC1 polypeptide, a GPC2 polypeptide, a MUC1 polypeptide, a CEACAM1 polypeptide, a CEACAM5 polypeptide (see, e.g., FIG. 17B), a CEACAM6 polypeptide, a CEACAM7 polypeptide, an EpCAM polypeptide, a CD19 polypeptide, a CD20 polypeptide, a CD22 polypeptide, a CD47 polypeptide, a VEGFR1 polypeptide, aVEGFR2 polypeptide, a CD30 polypeptide, a CD33 polypeptide, a KIT polypeptide, a FGFR polypeptide, a PDGFR polypeptide, a gp120 polypeptide (e.g., an HIV gp120 polypeptide), a gp160 polypeptide (e.g., an HIV gp120 polypeptide), or a RBD polypeptide (e.g., a SARS-CoV-2 RBD polypeptide). Examples of antigen binding domains having the ability to bind to a target antigen of interest expressed on the surface of a cell (e.g., a cancer cell and/or a virally infected cell) include, without limitation, those set forth in FIG. 20. In some cases, a binder such as the HIV binder depicted in FIG. 17E can be used to design a cell engager having the ability to bind to HIV coat proteins.

As described herein, at least one antigen binding domain of a cell engager provided herein can be designed to bind to a CD94/NKG2A polypeptide (e.g., a human CD94/NKG2A polypeptide) and at least another antigen binding domain of the cell engager provided herein can be designed to bind to target antigen of interest (e.g., a CEACAM5 polypeptide). For example, a cell engager provided herein can be designed to include the components of an antibody, antigen binding fragment, and/or antibody domain described herein (e.g., a combination of CDRs) as an antigen binding domain provided that that antigen binding domain has the ability to bind to a CD94/NKG2A polypeptide (e.g., a human CD94/NKG2A polypeptide). In some examples, a cell engager provided herein can be designed to include an antigen binding domain that includes two sets of three CDRs (e.g., CDR1, CDR2, and CDR3 of a heavy chain and CDR1, CDR2, and CDR3 of a light chain) of an antigen binding fragment provided herein (e.g., SEQ ID NOs:1-3 and 9-11 or SEQ ID NOs:17-19 and 25-27). In some cases, an antigen binding domain of a cell engager targeting a CD94/NKG2A polypeptide can be designed to include a VH domain described herein or a scFv/Fab antibody described herein. In some cases, an antigen binding domain of a CAR described herein that has the ability to bind to a CD94/NKG2A polypeptide (e.g., a human CD94/NKG2A polypeptide) can be used as an antigen binding domain of a cell engager that binds to a CD94/NKG2A⁺ cells (e.g., a CD94/NKG2A⁺ CD8⁺ T cell and/or a CD94/NKG2A⁺ NK cell).

As described herein, a cell engager can be designed to include at least one antigen binding domain having the ability to bind to a CD94/NKG2A polypeptide (e.g., a human CD94/NKG2A polypeptide) and at least one other antigen binding domain that binds to a target antigen of interest. In some cases, a cell engager can include an antigen binding domain having the ability to bind to a target antigen of interest expressed on the surface of a cell (e.g., a cancer cell and/or a virally infected cell), an antigen binding domain having the ability to bind to a CD94/NKG2A polypeptide (e.g., a human CD94/NKG2A polypeptide) expressed on the surface of a cell (e.g., a CD94/NKG2A⁺ CD8⁺ T cell and/or a CD94/NKG2A⁺ NK cell), and one or more other antigen binding domains (e.g., one, two, three, or four other antigen binding domains) where each of those other antigen binding domains can bind to different antigens expressed on the surface of different cell types or can bind to different antigens expressed on the surface of the same cell type. For example, a TriTE and/or TriKE can be designed to have a first antigen binding domain having the ability to bind to a target antigen of interest (e.g., a CEACAM5 polypeptide) expressed on the surface of a cell (e.g., a cancer cell and/or a virally infected cell), a second antigen binding domain having the ability to bind to a CD94/NKG2A polypeptide (e.g., a human CD94/NKG2A polypeptide) expressed on the surface of a T cell or NK cell (e.g., a CD94/NKG2A⁺ CD8⁺ T cell and/or a CD94/NKG2A⁺ NK cell), and a third antigen binding domain having the ability to bind to an antigen expressed on the surface of a T cell (e.g., a CD3 polypeptide) or an antigen expressed on the surface of an NK cell (e.g., a CD16 polypeptide such as a CD16a polypeptide).

In some cases, when designing a cell engager such as a TriTE to link a CD94/NKG2A⁺ T cell and a target cell expressing a target antigen of interest, the cell engager can include a first antigen binding domain having the ability to bind to a CD94/NKG2A polypeptide (e.g., a human CD94/NKG2A polypeptide) expressed on the surface of a T cell, a second antigen binding domain having the ability to bind to a polypeptide expressed on the surface of a T cell, and a third antigen binding domain having the ability to bind to a target antigen of interest expressed on the surface of a target cell (e.g., a cancer cell and/or a virally infected cell). Examples of polypeptides expressed on the surface of a T cell that can be targeted by an antigen binding domain of a cell engager provided herein include, without limitation, CD3 polypeptides. Examples of antigen binding domains having the ability to bind to a polypeptide expressed on the surface of a T cell that can be used to make a cell engager provided herein (e.g., a TriTE) include, without limitation, anti-CD3 scFvs and anti-CD3 VH domains. Additional examples of amino acid sequences that can be used as antigen binding domains having the ability to bind to a polypeptide expressed on the surface of a T cell (e.g., CD3) are described in U.S. Pat. No. 6,750,325 (see, e.g., the sequence listing of U.S. Pat. No. 6,750,325).

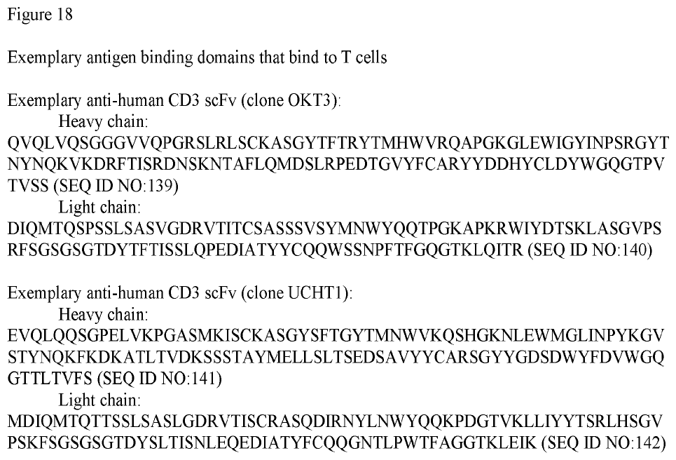

In some cases, a cell engager (e.g., a TriTE) provided herein can be designed to include an antigen binding domain that comprises, consists essentially of, or consists of one of the amino acid sequences set forth in FIG. 18. In some cases, a cell engager provided herein can be designed to include an antigen binding domain that comprises, consists essentially of, or consists of one of the amino acid sequences set forth in FIG. 18 with one, two, three, four, five, six, seven, eight, nine, or ten amino acid deletions, additions, substitutions, or combinations thereof, provided that the antigen binding domain has the ability to bind to a polypeptide expressed on the surface of a T cell. In some cases, a cell engager provided herein can be designed to include an antigen binding domain that comprises, consists essentially of, or consists of one of the amino acid sequences set forth in FIG. 18 with two or less, three or less, four or less, five or less, six or less, seven or less, eight or less, nine or less, or ten or less amino acid deletions, additions, substitutions, or combinations thereof, provided that the antigen binding domain has the ability to bind to a polypeptide expressed on the surface of a T cell.

In some cases, when designing a cell engager such as a TriKE to link a CD94/NKG2A⁺ NK cell and a target cell expressing a target antigen of interest, the cell engager can include a first antigen binding domain having the ability to bind to a CD94/NKG2A polypeptide (e.g., a human CD94/NKG2A polypeptide) expressed on the surface of an NK cell, a second antigen binding domain having the ability to bind to a polypeptide expressed on the surface of an NK cell, and a third antigen binding domain having the ability to bind to a target antigen of interest expressed on the surface of a target cell (e.g., a cancer cell and/or a virally infected cell). Examples of polypeptides expressed on the surface of an NK cell that can be targeted by an antigen binding domain of a cell engager provided herein include, without limitation, CD16 polypeptides (e.g., CD16a polypeptides), NKG2A polypeptides, NKG2D polypeptides, NKp30 polypeptides, NKp44 polypeptides, and NKp46 polypeptides. Examples of antigen binding domains having the ability to bind to a polypeptide expressed on the surface of an NK cell that can be used to make a cell engager provided herein (e.g., a TriKE) include, without limitation, anti-CD16a scFvs, anti-NKG2A scFvs, anti-NKG2D scFvs, anti-NKp30 scFvs (see, e.g., BioLegend Catalog #325207), anti-NKp44 scFvs, anti-NKp46 scFvs, anti-CD16a VH domains, anti-NKG2A VH domains, anti-NKG2D VH domains, anti-NKp30 VH domains, anti-NKp44 VH domains, and anti-NKp46 VH domains. Additional examples of amino acid sequences that can be used as antigen binding domains having the ability to bind to a polypeptide expressed on the surface of an NK cell (e.g., CD16, NKG2A, NKG2D, or NKp46) are described in McCall et al. (Mol. Immunol., 36(7):433-445 (1999); see, e.g., anti-CD16 scFv sequences); International Patent Application Publication No. PCT/US2017/048721 (see, e.g., the CDRs and sequence listing for anti-CD16a binding domains); U.S. Patent Application Publication No. 2011/0052606 (see, e.g., the CDRs and the sequence listing for anti-NKG2A antibodies such as Z199); U.S. Patent Application Publication No. 2011/0150870 (see, e.g., the CDRs and sequence listing for anti-NKG2D antibodies); U.S. Patent Application Publication No. 2018/0369373 (see, e.g., the CDRs and sequence listing for anti-NKp46 antibodies); and U.S. Patent Application Publication No. 2017/0368169 (see, e.g., the CDRs and sequence listing for anti-NKp46 antibodies).

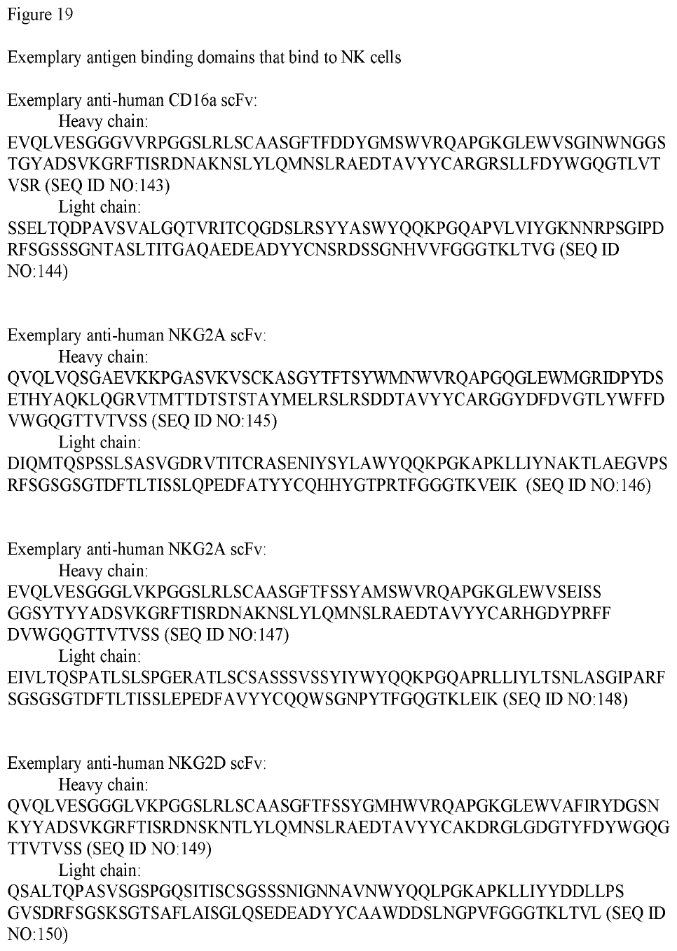

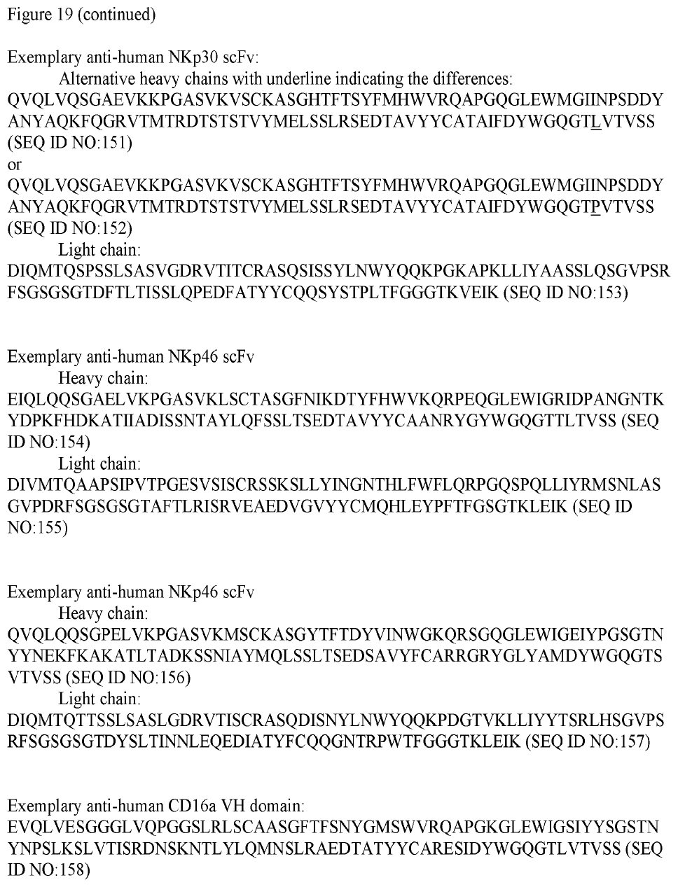

In some cases, a cell engager (e.g., a TriKE) provided herein can be designed to include an antigen binding domain (e.g., a scFv or VH) that comprises, consists essentially of, or consists of one or more of the amino acid sequences set forth in FIG. 19. In some cases, a cell engager provided herein can be designed to include an antigen binding domain (e.g., a scFv or VH) that comprises, consists essentially of, or consists of one of the amino acid sequences set forth in FIG. 19 with one, two, three, four, five, six, seven, eight, nine, or ten amino acid deletions, additions, substitutions, or combinations thereof, provided that the antigen binding domain has the ability to bind to a polypeptide expressed on the surface of an NK cell. In some cases, a cell engager provided herein can be designed to include an antigen binding domain (e.g., a scFv or VH) that comprises, consists essentially of, or consists of one of the amino acid sequences set forth in FIG. 19 with two or less, three or less, four or less, five or less, six or less, seven or less, eight or less, nine or less, or ten or less amino acid deletions, additions, substitutions, or combinations thereof, provided that the antigen binding domain has the ability to bind to a polypeptide expressed on the surface of an NK cell.

In some cases, a cell engager provided herein can be designed to include a linker located between each antigen binding domain. Any appropriate linker can be used to design a cell engager provided herein. Examples of linkers that can be used to make a cell engager described herein include, without limitation, the linker sequences set forth in FIG. 10. A cell engager provided herein can be designed to include a linker of any appropriate length. For example, a cell engager provided herein can be designed to include a linker that is from about 3 to about 100 (e.g., from about 3 to about 90, from about 3 to about 80, from about 3 to about 70, from about 3 to about 60, from about 3 to about 50, from about 3 to about 40, from about 3 to about 30, from about 3 to about 20, from about 3 to about 15, from about 5 to about 100, from about 10 to about 100, from about 20 to about 100, from about 30 to about 100, from about 40 to about 100, from about 50 to about 100, from about 60 to about 100, from about 70 to about 100, from about 10 to about 50, from about 10 to about 40, from about 10 to about 30, from about 10 to about 20, or from about 12 to about 17) amino acid residues in length. In some cases, a cell engager provided herein (e.g., a BiTE) can be designed to include a GGGGSGGGGSGGGGS (SEQ ID NO:78) linker. In some cases, a hinge of a CAR described herein can be used as a linker to make a cell engager described herein. For example, any one of the sequences set forth in FIG. 13 can be used as a linker of a cell engager described herein.

In some cases, a cell engager provided herein can be designed to include a linker that comprises, consists essentially of, or consists of one of the amino acid sequences set forth in FIG. 10 or FIG. 13. In some cases, a cell engager provided herein can be designed to include a linker that comprises, consists essentially of, or consists of one of the amino acid sequences set forth in FIG. 10 or FIG. 13 with one, two, three, four, five, six, seven, eight, nine, or ten amino acid deletions, additions, substitutions, or combinations thereof. In some cases, a cell engager provided herein can be designed to include a linker that comprises, consists essentially of, or consists of one of the amino acid sequences set forth in FIG. 10 or FIG. 13 with two or less, three or less, four or less, five or less, six or less, seven or less, eight or less, nine or less, or ten or less amino acid deletions, additions, substitutions, or combinations thereof.

In some cases, a cell engager (e.g., a BiTE, BiKE, TriTE, and/or TriKE) targeting an antigen of interest can be designed to include an scFv having a heavy chain variable domain comprising SEQ ID NO:1, SEQ ID NO:2, and SEQ ID NO:3, followed by an optional linker such as a linker set forth in FIG. 10, followed by a light chain variable domain comprising SEQ ID NO:9, SEQ ID NO:10, and SEQ ID NO:11, followed by an optional linker such as a hinge/linker set forth in FIG. 10 or FIG. 13 (e.g., SEQ ID NO:78), followed by an antigen binding domain having the ability to bind to an antigen of interest expressed on the surface of a cell (e.g., a cancer cell and/or a virally infected cell).

In some cases, a cell engager (e.g., a BiTE, BiKE, TriTE, and/or TriKE) targeting an antigen of interest can be designed to include an scFv having a light chain variable domain comprising SEQ ID NO:9, SEQ ID NO:10, and SEQ ID NO:11, followed by an optional linker such as a linker set forth in FIG. 10, followed by a heavy chain variable domain comprising SEQ ID NO:1, SEQ ID NO:2, and SEQ ID NO:3, followed by an optional linker such as a hinge/linker set forth in FIG. 10 or FIG. 13 (e.g., SEQ ID NO:78), followed by an antigen binding domain having the ability to bind to an antigen of interest expressed on the surface of a cell (e.g., a cancer cell and/or a virally infected cell).

In some cases, a cell engager (e.g., a BiTE, BiKE, TriTE, and/or TriKE) targeting an antigen of interest can be designed to include an scFv having a heavy chain variable domain comprising SEQ ID NO:8, followed by an optional linker such as a linker set forth in FIG. 10, followed by a light chain variable domain comprising SEQ ID NO:16, followed by an optional linker such as a hinge/linker set forth in FIG. 10 or FIG. 13 (e.g., SEQ ID NO:78), followed by an antigen binding domain having the ability to bind to an antigen of interest expressed on the surface of a cell (e.g., a cancer cell and/or a virally infected cell).

In some cases, a cell engager (e.g., a BiTE, BiKE, TriTE, and/or TriKE) targeting an antigen of interest can be designed to include an scFv having a light chain variable domain comprising SEQ ID NO:16, followed by a linker such as an optional linker set forth in FIG. 10, followed by a heavy chain variable domain comprising SEQ ID NO:8, followed by an optional linker such as a hinge/linker set forth in FIG. 10 or FIG. 13 (e.g., SEQ ID NO:78), followed by an antigen binding domain having the ability to bind to an antigen of interest expressed on the surface of a cell (e.g., a cancer cell and/or a virally infected cell).

In some cases, a cell engager (e.g., a BiTE, BiKE, TriTE, and/or TriKE) targeting an antigen of interest can be designed to include an scFv having a heavy chain variable domain comprising SEQ ID NO:17, SEQ ID NO:18, and SEQ ID NO:19, followed by an optional linker such as a linker set forth in FIG. 10, followed by a light chain variable domain comprising SEQ ID NO:25, SEQ ID NO:26, and SEQ ID NO:27, followed by an optional linker such as a hinge/linker set forth in FIG. 10 or FIG. 13 (e.g., SEQ ID NO:78), followed by an antigen binding domain having the ability to bind to an antigen of interest expressed on the surface of a cell (e.g., a cancer cell and/or a virally infected cell).

In some cases, a cell engager (e.g., a BiTE, BiKE, TriTE, and/or TriKE) targeting an antigen of interest can be designed to include an scFv having a light chain variable domain comprising SEQ ID NO:25, SEQ ID NO:26, and SEQ ID NO:27, followed by an optional linker such as a linker set forth in FIG. 10, followed by a heavy chain variable domain comprising SEQ ID NO:17, SEQ ID NO:18, and SEQ ID NO:19, followed by an optional linker such as a hinge/linker set forth in FIG. 10 or FIG. 13 (e.g., SEQ ID NO:78), followed by an antigen binding domain having the ability to bind to an antigen of interest expressed on the surface of a cell (e.g., a cancer cell and/or a virally infected cell).

In some cases, a cell engager (e.g., a BiTE, BiKE, TriTE, and/or TriKE) targeting an antigen of interest can be designed to include an scFv having a heavy chain variable domain comprising SEQ ID NO:24, followed by an optional linker such as a linker set forth in FIG. 10, followed by a light chain variable domain comprising SEQ ID NO:32, followed by an optional linker such as a hinge/linker set forth in FIG. 10 or FIG. 13 (e.g., SEQ ID NO:78), followed by an antigen binding domain having the ability to bind to an antigen of interest expressed on the surface of a cell (e.g., a cancer cell and/or a virally infected cell).

In some cases, a cell engager (e.g., a BiTE, BiKE, TriTE, and/or TriKE) targeting an antigen of interest can be designed to include an scFv having a light chain variable domain comprising SEQ ID NO:32, followed by an optional linker such as a linker set forth in FIG. 10, followed by a heavy chain variable domain comprising SEQ ID NO:24, followed by an optional linker such as a hinge/linker set forth in FIG. 10 or FIG. 13 (e.g., SEQ ID NO:78), followed by an antigen binding domain having the ability to bind to an antigen of interest expressed on the surface of a cell (e.g., a cancer cell and/or a virally infected cell).

In some cases, a cell engager (e.g., a BiTE, BiKE, TriTE, and/or TriKE) targeting an antigen of interest can be designed to include an IgG (e.g., IgG1) configuration having (a) a heavy chain comprising, consisting essentially of, or consisting of a heavy chain variable domain comprising SEQ ID NO:1, SEQ ID NO:2, and SEQ ID NO:3, an Ig hinge, and constant domains (e.g., CH1, CH2, and CH3 domains) and (b) a light chain comprising, consisting essentially of, or consisting of a light chain variable domain comprising SEQ ID NO:9, SEQ ID NO:10, and SEQ ID NO:11, a constant domain (e.g., a kappa or lambda constant domain), and an antigen binding domain having the ability to bind to an antigen of interest expressed on the surface of a cell (e.g., a cancer cell and/or a virally infected cell).

In some cases, a cell engager (e.g., a BiTE, BiKE, TriTE, and/or TriKE) targeting an antigen of interest can be designed to include an IgG (e.g., IgG1) configuration having (a) a heavy chain comprising, consisting essentially of, or consisting of a heavy chain variable domain comprising SEQ ID NO:8, an Ig hinge, and constant domains (e.g., CH1, CH2, and CH3 domains) and (b) a light chain comprising, consisting essentially of, or consisting of a light chain variable domain comprising SEQ ID NO:16, a constant domain (e.g., a kappa or lambda constant domain), and an antigen binding domain having the ability to bind to an antigen of interest expressed on the surface of a cell (e.g., a cancer cell and/or a virally infected cell).

In some cases, a cell engager (e.g., a BiTE, BiKE, TriTE, and/or TriKE) targeting an antigen of interest can be designed to include an IgG (e.g., IgG1) configuration having (a) a heavy chain comprising, consisting essentially of, or consisting of a heavy chain variable domain comprising SEQ ID NO:17, SEQ ID NO:18, and SEQ ID NO:19, an Ig hinge, and constant domains (e.g., CH1, CH2, and CH3 domains) and (b) a light chain comprising, consisting essentially of, or consisting of a light chain variable domain comprising SEQ ID NO:25, SEQ ID NO:26, and SEQ ID NO:27, a constant domain (e.g., a kappa or lambda constant domain), and an antigen binding domain having the ability to bind to an antigen of interest expressed on the surface of a cell (e.g., a cancer cell and/or a virally infected cell).

In some cases, a cell engager (e.g., a BiTE, BiKE, TriTE, and/or TriKE) targeting an antigen of interest can be designed to include an IgG (e.g., IgG1) configuration having (a) a heavy chain comprising, consisting essentially of, or consisting of a heavy chain variable domain comprising SEQ ID NO:24, an Ig hinge, and constant domains (e.g., CH1, CH2, and CH3 domains) and (b) a light chain comprising, consisting essentially of, or consisting of a light chain variable domain comprising SEQ ID NO:32, a constant domain (e.g., a kappa or lambda constant domain), and an antigen binding domain having the ability to bind to an antigen of interest expressed on the surface of a cell (e.g., a cancer cell and/or a virally infected cell).

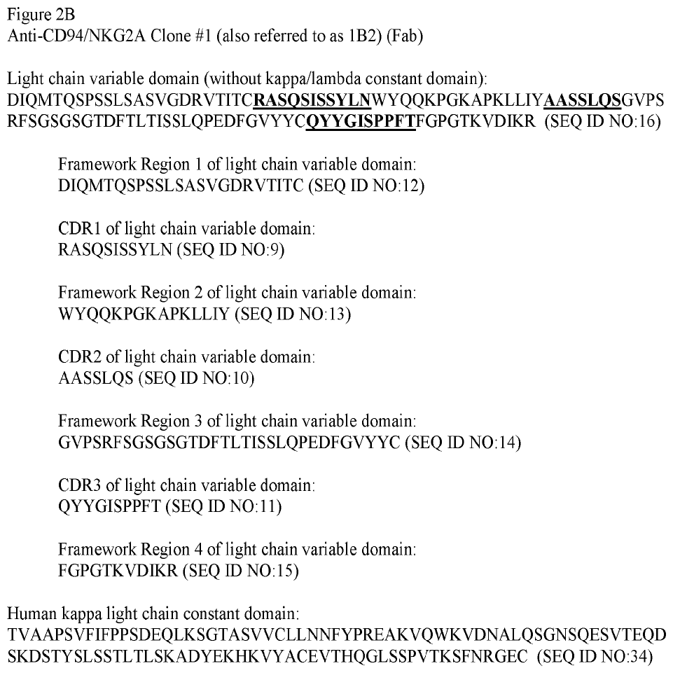

In one embodiment, a binder (e.g., an antibody, antigen binding fragment, antibody domain, a CAR, a cell engager, and/or an ADC) provided herein having the ability to bind to a CD94/NKG2A polypeptide (e.g., a human CD94/NKG2A polypeptide) can include (i) a heavy chain variable domain having a CDR1 having the amino acid sequence set forth in SEQ ID NO:1 (or a variant of SEQ ID NO:1 with one or two amino acid modifications), a CDR2 having the amino acid sequence set forth in SEQ ID NO:2 (or a variant of SEQ ID NO:2 with one or two amino acid modifications), and a CDR3 having the amino acid sequence set forth in SEQ ID NO:3 (or a variant of SEQ ID NO:3 with one or two amino acid modifications); and/or (ii) a light chain variable domain having a CDR1 having the amino acid sequence set forth in SEQ ID NO:9 (or a variant of SEQ ID NO:9 with one or two amino acid modifications), a CDR2 having the amino acid sequence set forth in SEQ ID NO:10 (or a variant of SEQ ID NO:10 with one or two amino acid modifications), and a CDR3 having the amino acid sequence set forth SEQ ID NO:11 (or a variant of SEQ ID NO:11 with one or two amino acid modifications). An example of such an antigen binding fragment having these CDRs and the ability to bind to a CD94/NKG2A polypeptide (e.g., a human CD94/NKG2A polypeptide) includes, without limitation, the Fab set forth in FIGS. 2A and 2B.

In some cases, a binder (e.g., an antibody, antigen binding fragment, antibody domain, a CAR, a cell engager, and/or an ADC) provided herein having the ability to bind to a CD94/NKG2A polypeptide (e.g., a human CD94/NKG2A polypeptide) and (a) a heavy chain variable domain having a CDR1 having the amino acid sequence set forth in SEQ ID NO:1 (or a variant of SEQ ID NO:1 with one or two amino acid modifications), a CDR2 having the amino acid sequence set forth in SEQ ID NO:2 (or a variant of SEQ ID NO:2 with one or two amino acid modifications), and a CDR3 having the amino acid sequence set forth in SEQ ID NO:3 (or a variant of SEQ ID NO:3 with one or two amino acid modifications) and/or (b) a light chain variable domain having a CDR1 having the amino acid sequence set forth in SEQ ID NO:9 (or a variant of SEQ ID NO:9 with one or two amino acid modifications), a CDR2 having the amino acid sequence set forth in SEQ ID NO:10 (or a variant of SEQ ID NO:10 with one or two amino acid modifications), and a CDR3 having the amino acid sequence set forth SEQ ID NO:11 (or a variant of SEQ ID NO:11 with one or two amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:4 (or a variant of SEQ ID NO:4 with one, two, three, four, five, six, seven, eight, nine, ten, or more amino acid modifications), a framework region 2 having the amino acid sequence set forth in SEQ ID NO:5 (or a variant of SEQ ID NO:5 with one, two, three, four, five, six, seven, eight, nine, ten, or more amino acid modifications), a framework region 3 having the amino acid sequence set forth in SEQ ID NO:6 (or a variant of SEQ ID NO:6 with one, two, three, four, five, six, seven, eight, nine, ten, or more amino acid modifications), and a framework region 4 having the amino acid sequence set forth in SEQ ID NO:7 (or a variant of SEQ ID NO:7 with one, two, three, four, five, six, seven, eight, nine, ten, or more amino acid modifications) and/or (b) a light chain variable domain that includes a framework region 1 having the amino acid sequence set forth in SEQ ID NO:12 (or a variant of SEQ ID NO:12 with one, two, three, four, five, six, seven, eight, nine, ten, or more amino acid modifications), a framework region 2 having the amino acid sequence set forth in SEQ ID NO:13 (or a variant of SEQ ID NO:13 with one, two, three, four, five, six, seven, eight, nine, ten, or more amino acid modifications), a framework region 3 having the amino acid sequence set forth in SEQ ID NO:14 (or a variant of SEQ ID NO:14 with one, two, three, four, five, six, seven, eight, nine, ten, or more amino acid modifications), and a framework region 4 having the amino acid sequence set forth in SEQ ID NO:15 (or a variant of SEQ ID NO:15 with one, two, three, four, five, six, seven, eight, nine, ten, or more amino acid modifications).

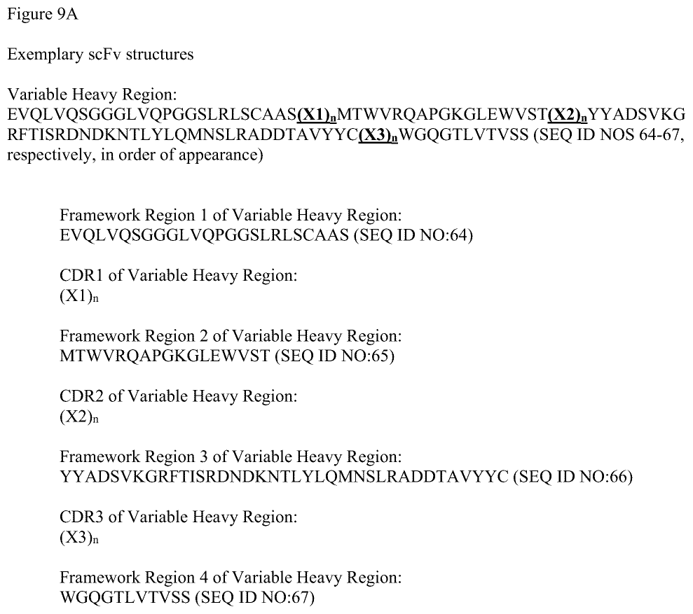

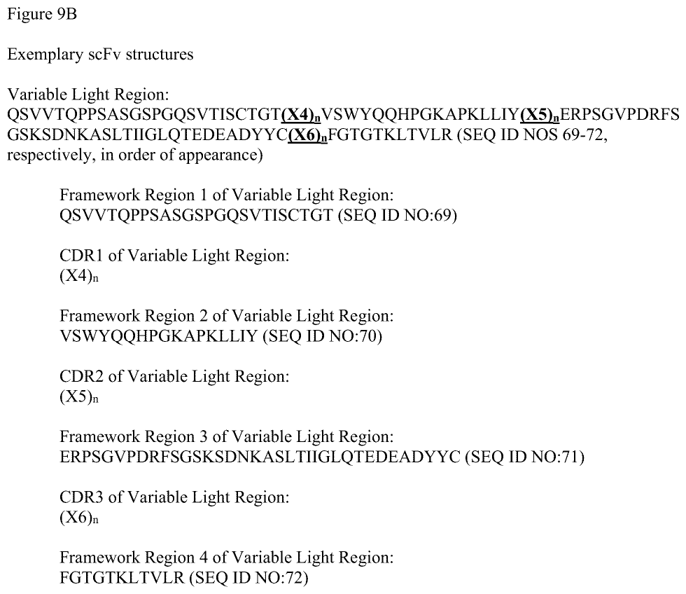

In some cases, a binder (e.g., an antibody, antigen binding fragment, antibody domain, a CAR, a cell engager, and/or an ADC) having any of the CDRs set forth in FIG. 2A or 2B can be designed to include framework regions as set forth in FIGS. 2A and 2B or can be designed to include one or more framework regions from another antibody, antibody fragment, or antibody domain. For example, an Fab can be designed to include the six CDRs set forth in FIGS. 2A and 2B and the framework regions set forth in FIGS. 2A and 2B except that framework region 1 having the amino acid set forth in SEQ ID NO:4 is replaced with a framework region 1 having the amino acid set forth in SEQ ID NO:20 or a framework region 1 having the amino acid set forth in SEQ ID NO:54, or a framework region 1 having the amino acid set forth in SEQ ID NO:64. In some cases, a scFv can be designed to include the six CDRs set forth in FIGS. 2A and 2B and the framework regions set forth in FIGS. 2A and 2B. In some cases, a scFv can be designed to include the six CDRs set forth in FIGS. 2A and 2B and the framework regions set forth in FIGS. 2A and 2B except that framework region 1 having the amino acid set forth in SEQ ID NO:4 is replaced with a framework region 1 having the amino acid set forth in SEQ ID NO:20, a framework region 1 having the amino acid set forth in SEQ ID NO:54, or a framework region 1 having the amino acid set forth in SEQ ID NO:64. In another example, a scFv can be designed to include the six CDRs set forth in FIGS. 2A and 2B and the framework regions set forth in FIGS. 6B and 6C, FIGS. 7A and 7B, FIGS. 8A and 8B, or FIGS. 9A and 9B.

In some cases, a binder (e.g., an antibody, antigen binding fragment, antibody domain, a CAR, a cell engager, and/or an ADC) provided herein having the ability to bind to a CD94/NKG2A polypeptide (e.g., a human CD94/NKG2A polypeptide) can include (a) a heavy chain variable domain that includes an amino acid sequence having at least 90 percent identity to the amino acid sequence set forth in SEQ ID NO:8 and/or (b) a light chain variable domain that includes an amino acid sequence having at least 90 percent identity to the amino acid sequence set forth in SEQ ID NO:16. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:8 and/or (b) a light chain variable domain that includes an amino acid sequence having at least 90, 91, 92, 93, 94, 95, 96, 97, 98, or 99 percent identity to the amino acid sequence set forth in SEQ ID NO:16. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:8 and/or (b) a light chain variable domain that includes an amino acid sequence having 100 percent identity to the amino acid sequence set forth in SEQ ID NO:16.

In some cases, a binder (e.g., an antibody, antigen binding fragment, antibody domain, a CAR, a cell engager, and/or an ADC) provided herein having the ability to bind to a CD94/NKG2A polypeptide (e.g., a human CD94/NKG2A polypeptide) can include (a) a heavy chain variable domain that includes an amino acid sequence having at least 90 percent identity to the amino acid sequence set forth in SEQ ID NO:8, provided that the heavy chain variable domain includes the amino acid sequences set forth in SEQ ID NOs:1, 2, and 3, and/or (b) a light chain variable domain that includes an amino acid sequence having at least 90 percent identity to the amino acid sequence set forth in SEQ ID NO:16, provided that the light chain variable domain includes the amino acid sequences set forth in SEQ ID NOs:9, 10, and 11. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:8, provided that the heavy chain variable domain includes the amino acid sequences set forth in SEQ ID NOs:1, 2, and 3, and/or (b) a light chain variable domain that includes an amino acid sequence having at least 90, 91, 92, 93, 94, 95, 96, 97, 98, or 99 percent identity to the amino acid sequence set forth in SEQ ID NO:16, provided that the light chain variable domain includes the amino acid sequences set forth in SEQ ID NOs:9, 10, and 11.

In some cases, a binder (e.g., an antibody, antigen binding fragment, antibody domain, a CAR, a cell engager, and/or an ADC) provided herein having the ability to bind to a CD94/NKG2A polypeptide (e.g., a human CD94/NKG2A polypeptide) can include (a) a heavy chain variable domain having the amino acid sequence set forth in SEQ ID NO:8 or the amino acid set forth in SEQ ID NO:8 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:16 or the amino acid set forth in SEQ ID NO:16 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to a CD94/NKG2A polypeptide (e.g., a human CD94/NKG2A polypeptide), can include a heavy chain variable domain having the amino acid sequence set forth in SEQ ID NO:8 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:1, 2, and 3, and can include a light chain variable domain having the amino acid sequence set forth in SEQ ID NO:16 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs:9, 10, and 11.

In some cases, a binder (e.g., an antibody, antigen binding fragment, antibody domain, a CAR, a cell engager, and/or an ADC) provided herein having the ability to bind to a CD94/NKG2A polypeptide (e.g., a human CD94/NKG2A polypeptide) can include (a) a heavy chain variable domain comprising (i) a CDR1 that comprises, consists essentially of, or consists of the amino acid sequence set forth in SEQ ID NO: 1, (ii) a CDR2 that comprises, consists essentially of, or consists of the amino acid sequence set forth in SEQ ID NO:2, and (iii) a CDR3 that comprises, consists essentially of, or consists of the amino acid sequence set forth in SEQ ID NO:3, and/or (b) a light chain variable domain comprising (i) a CDR1 that comprises, consists essentially of, or consists of the amino acid sequence set forth in SEQ ID NO:9, (ii) a CDR2 that comprises, consists essentially of, or consists of the amino acid sequence set forth in SEQ ID NO:10, and (iii) a CDR3 that comprises, consists essentially of, or consists of the amino acid sequence set forth in SEQ ID NO:11. As used herein, a “CDR1 that consists essentially of the amino acid sequence set forth in SEQ ID NO:1” is a CDR1 that has zero, one, or two amino acid substitutions within SEQ ID NO:1, that has zero, one, two, three, four, or five amino acid residues directly preceding SEQ ID NO:1, and/or that has zero, one, two, three, four, or five amino acid residues directly following SEQ ID NO:1, provided that the binder (e.g., an antibody, antigen binding fragment, antibody domain, a CAR, a cell engager, and/or an ADC) maintains its basic ability to bind to a CD94/NKG2A polypeptide (e.g., a human CD94/NKG2A polypeptide). Examples of a CDR1 that consists essentially of the amino acid sequence set forth in SEQ ID NO:1 include, without limitation, those set forth in Table 1.

TABLE 1
Exemplary CDR1s that consist essentially
of the amino acid sequence set forth in
SEQ ID NO: 1.

| Sequence | SEQ ID NO: |
|---|---|
| SYYMS | 166 |
| DYAMS | 167 |
| DYYTS | 168 |
| DYYMH | 169 |
| NYYMS | 170 |
| DNYMS | 171 |
| DYGMS | 172 |
| DYYMN | 173 |
| GYYMS | 174 |
| DAYMS | 175 |

As used herein, a “CDR2 that consists essentially of the amino acid sequence set forth in SEQ ID NO:2” is a CDR2 that has zero, one, or two amino acid substitutions within SEQ ID NO:2, that has zero, one, two, three, four, or five amino acid residues directly preceding SEQ ID NO:2, and/or that has zero, one, two, three, four, or five amino acid residues directly following SEQ ID NO:2, provided that the binder (e.g., an antibody, antigen binding fragment, antibody domain, a CAR, a cell engager, and/or an ADC) maintains its basic ability to bind to a CD94/NKG2A polypeptide (e.g., a human CD94/NKG2A polypeptide). Examples of a CDR2 that consists essentially of the amino acid sequence set forth in SEQ ID NO:2 include, without limitation, those set forth in Table 2.

TABLE 2
Exemplary CDR2s that consist essentially
of the amino acid sequence set forth in
SEQ ID NO: 2.

| Sequence | SEQ ID NO: |
|---|---|
| VISSSGSTIYYADSVKG | 176 |
| YVSSGSTIYYADSVKG | 177 |
| YIGSSGSTIYYADSVKG | 178 |
| YISYSGSTIYYADSVKG | 179 |
| YISSDGSTIYYADSVKG | 180 |
| YISSSSSTIYYADSVKG | 181 |
| YISSSGGTIYYADSVKG | 182 |
| YISSSGSSIYYADSVKG | 183 |
| YISSSGSTTYYADSVKG | 184 |
| YISSSGSTIHYADSVKG | 185 |

As used herein, a “CDR3 that consists essentially of the amino acid sequence set forth in SEQ ID NO:3” is a CDR3 that has zero, one, or two amino acid substitutions within SEQ TD NO:3, that has zero, one, two, three, four, or five amino acid residues directly preceding SEQ ID NO:3, and/or that has zero, one, two, three, four, or five amino acid residues directly following SEQ ID NO:3, provided that the binder (e.g., an antibody, antigen binding fragment, antibody domain, a CAR, a cell engager, and/or an ADC) maintains its basic ability to bind to a CD94/NKG2A polypeptide (e.g., a human CD94/NKG2A polypeptide). Examples of a CDR3 that consists essentially of the amino acid sequence set forth in SEQ ID NO:3 include, without limitation, those set forth in Table 3.

TABLE 3
Exemplary CDR3s that consist essentially
of the amino acid sequence set forth in
SEQ ID NO: 3.

| Sequence | SEQ ID NO: |
|---|---|
| GWYMDA | 186 |
| GYYMDA | 187 |
| GLYMDA | 188 |
| GIYMDA | 189 |
| SFYMDA | 190 |
| AFYMDA | 191 |
| VFYMDA | 192 |
| GHYMDA | 193 |
| GWHMDA | 194 |
| GWMMDA | 195 |

As used herein, a “CDR1 that consists essentially of the amino acid sequence set forth in SEQ ID NO:9” is a CDR1 that has zero, one, or two amino acid substitutions within SEQ TD NO:9, that has zero, one, two, three, four, or five amino acid residues directly preceding SEQ ID NO:9, and/or that has zero, one, two, three, four, or five amino acid residues directly following SEQ ID NO:9, provided that the binder (e.g., an antibody, antigen binding fragment, antibody domain, a CAR, a cell engager, and/or an ADC) maintains its basic ability to bind to a CD94/NKG2A polypeptide (e.g., a human CD94/NKG2A polypeptide). Examples of a CDR1 that consists essentially of the amino acid sequence set forth in SEQ ID NO:9 include, without limitation, those set forth in Table 4.

TABLE 4
Exemplary CDR1s that consist essentially
of the amino acid sequence set forth in
SEQ ID NO: 9.

| Sequence | SEQ ID NO: |
|---|---|
| QASQSISSYLN | 196 |
| RMSQSISSYLN | 197 |
| RARQSISSYLN | 198 |
| RASESISSYLN | 199 |
| RASQGISSYLN | 200 |
| RASQSIRSYLN | 201 |
| RASQSISNYLN | 202 |
| RASQSISSWLN | 203 |
| RASQSISSYLA | 204 |
| WASQSISSYLN | 205 |

As used herein, a “CDR2 that consists essentially of the amino acid sequence set forth in SEQ ID NO:10” is a CDR2 that has zero, one, or two amino acid substitutions within SEQ TD NO:10, that has zero, one, two, three, four, or five amino acid residues directly preceding SEQ ID NO:10, and/or that has zero, one, two, three, four, or five amino acid residues directly following SEQ ID NO:10, provided that the binder (e.g., an antibody, antigen binding fragment, antibody domain, a CAR, a cell engager, and/or an ADC) maintains its basic ability to bind to a CD94/NKG2A polypeptide (e.g., a human CD94/NKG2A polypeptide). Examples of a CDR2 that consists essentially of the amino acid sequence set forth in SEQ ID NO:10 include, without limitation, those set forth in Table 5.

TABLE 5
Exemplary CDR2s that consist essentially
of the amino acid sequence set forth in
SEQ ID NO: 10.

| Sequence | SEQ ID NO: |
|---|---|
| DASSLQS | 206 |
| AAKSLQS | 207 |
| AASTLQS | 208 |
| AASSLES | 209 |
| AASSLQT | 210 |
| SASSLQS | 211 |
| AAPSLQS | 212 |
| AASNLQS | 213 |
| AASSLHS | 214 |
| AASSLQP | 215 |

As used herein, a “CDR3 that consists essentially of the amino acid sequence set forth in SEQ ID NO:11” is a CDR3 that has zero, one, or two amino acid substitutions within SEQ TD NO:11, that has zero, one, two, three, four, or five amino acid residues directly preceding SEQ ID NO:11, and/or that has zero, one, two, three, four, or five amino acid residues directly following SEQ ID NO:11, provided that the binder (e.g., an antibody, antigen binding fragment, antibody domain, a CAR, a cell engager, and/or an ADC) maintains its basic ability to bind to a CD94/NKG2A polypeptide (e.g., a human CD94/NKG2A polypeptide). Examples of a CDR3 that consists essentially of the amino acid sequence set forth in SEQ ID NO:11 include, without limitation, those set forth in Table 6.

TABLE 6
Exemplary CDR3s that consist essentially
of the amino acid sequence set forth in
SEQ ID NO: 11.

| Sequence | SEQ ID NO: |
|---|---|
| QQYGISPPFT | 216 |
| QHYGISPPFT | 217 |
| QYHGISPPFT | 218 |
| QSYGISPPFT | 219 |
| QYSGISPPFT | 220 |
| QYYAISPPFT | 221 |
| QYYSISPPFT | 222 |
| QYYGITPPFT | 223 |
| QYYTISPPFT | 224 |
| QYYGIYPPFT | 225 |

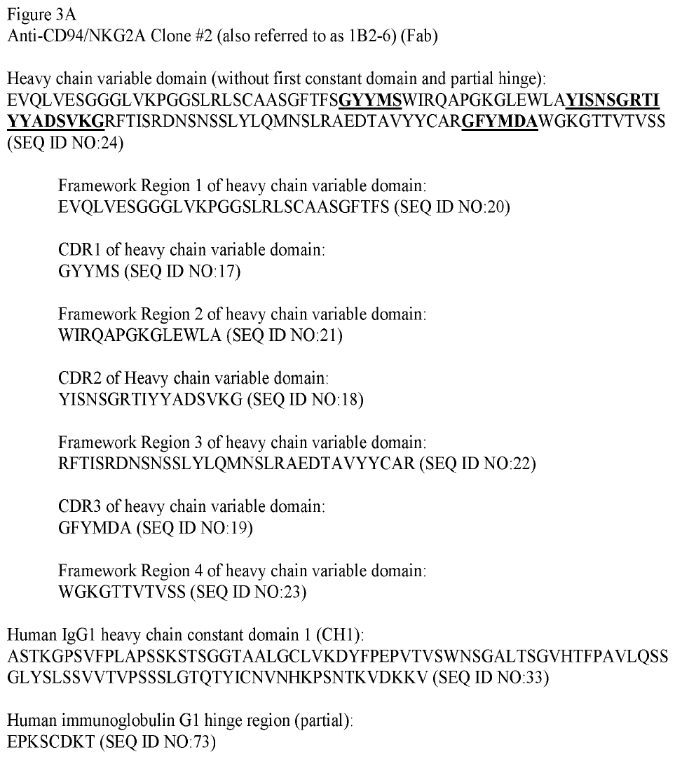

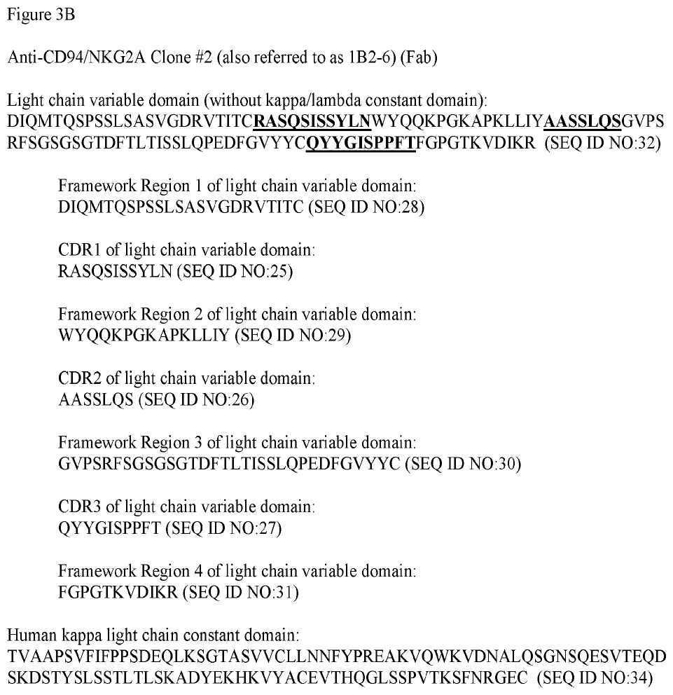

In another embodiment, a binder (e.g., an antibody, antigen binding fragment, antibody domain, a CAR, a cell engager, and/or an ADC) provided herein having the ability to bind to a CD94/NKG2A polypeptide (e.g., a human CD94/NKG2A polypeptide) can include (i) a heavy chain variable domain having a CDR1 having the amino acid sequence set forth in SEQ ID NO:17 (or a variant of SEQ ID NO:17 with one or two amino acid modifications), a CDR2 having the amino acid sequence set forth in SEQ ID NO:18 (or a variant of SEQ ID NO:18 with one or two amino acid modifications), and a CDR3 having the amino acid sequence set forth in SEQ ID NO:19 (or a variant of SEQ ID NO:19 with one or two amino acid modifications); and/or (ii) a light chain variable domain having a CDR1 having the amino acid sequence set forth in SEQ ID NO:25 (or a variant of SEQ ID NO:25 with one or two amino acid modifications), a CDR2 having the amino acid sequence set forth in SEQ ID NO:26 (or a variant of SEQ ID NO:26 with one or two amino acid modifications), and a CDR3 having the amino acid sequence set forth SEQ ID NO:27 (or a variant of SEQ ID NO:27 with one or two amino acid modifications). An example of such an antigen binding fragment having these CDRs and the ability to bind to a CD94/NKG2A polypeptide (e.g., a human CD94/NKG2A polypeptide) includes, without limitation, the Fab set forth in FIGS. 3A and 3B.

In some cases, a binder (e.g., an antibody, antigen binding fragment, antibody domain, a CAR, a cell engager, and/or an ADC) provided herein having the ability to bind to a CD94/NKG2A polypeptide (e.g., a human CD94/NKG2A polypeptide) and having (a) a heavy chain variable domain having a CDR1 having the amino acid sequence set forth in SEQ ID NO:17 (or a variant of SEQ ID NO:17 with one or two amino acid modifications), a CDR2 having the amino acid sequence set forth in SEQ ID NO:18 (or a variant of SEQ ID NO:18 with one or two amino acid modifications), and a CDR3 having the amino acid sequence set forth in SEQ ID NO:19 (or a variant of SEQ ID NO:19 with one or two amino acid modifications) and/or (b) a light chain variable domain having a CDR1 having the amino acid sequence set forth in SEQ ID NO:25 (or a variant of SEQ ID NO:25 with one or two amino acid modifications), a CDR2 having the amino acid sequence set forth in SEQ ID NO:26 (or a variant of SEQ ID NO:26 with one or two amino acid modifications), and a CDR3 having the amino acid sequence set forth SEQ ID NO:27 (or a variant of SEQ ID NO:27 with one or two amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:20 (or a variant of SEQ ID NO:20 with one, two, three, four, five, six, seven, eight, nine, ten, or more amino acid modifications), a framework region 2 having the amino acid sequence set forth in SEQ ID NO:21 (or a variant of SEQ ID NO:21 with one, two, three, four, five, six, seven, eight, nine, ten, or more amino acid modifications), a framework region 3 having the amino acid sequence set forth in SEQ ID NO:22 (or a variant of SEQ ID NO:22 with one, two, three, four, five, six, seven, eight, nine, ten, or more amino acid modifications), and a framework region 4 having the amino acid sequence set forth in SEQ ID NO:23 (or a variant of SEQ ID NO:23 with one, two, three, four, five, six, seven, eight, nine, ten, or more amino acid modifications) and/or (b) a light chain variable domain that includes a framework region 1 having the amino acid sequence set forth in SEQ ID NO:28 (or a variant of SEQ ID NO:28 with one, two, three, four, five, six, seven, eight, nine, ten, or more amino acid modifications), a framework region 2 having the amino acid sequence set forth in SEQ ID NO:29 (or a variant of SEQ ID NO:29 with one, two, three, four, five, six, seven, eight, nine, ten, or more amino acid modifications), a framework region 3 having the amino acid sequence set forth in SEQ ID NO:30 (or a variant of SEQ ID NO:30 with one, two, three, four, five, six, seven, eight, nine, ten, or more amino acid modifications), and a framework region 4 having the amino acid sequence set forth in SEQ ID NO:31 (or a variant of SEQ ID NO:31 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 3A or 3B can be designed to include framework regions as set forth in FIGS. 3A and 3B or can be designed to include one or more framework regions from another antibody or antibody fragment. For example, an Fab can be designed to include the six CDRs set forth in FIGS. 3A and 3B and the framework regions set forth in FIGS. 3A and 3B except that framework region 1 having the amino acid set forth in SEQ ID NO:20 is replaced with a framework region 1 having the amino acid set forth in SEQ ID NO:4 or a framework region 1 having the amino acid set forth in SEQ ID NO:54 or a framework region 1 having the amino acid set forth in SEQ ID NO:64. In some cases, a scFv can be designed to include the six CDRs set forth in FIGS. 3A and 3B and the framework regions set forth in FIGS. 3A and 3B. In some cases, a scFv can be designed to include the six CDRs set forth in FIGS. 3A and 3B and the framework regions set forth in FIGS. 3A and 3B except that framework region 1 having the amino acid set forth in SEQ ID NO:20 is replaced with a framework region 1 having the amino acid set forth in SEQ ID NO:4 or a framework region 1 having the amino acid set forth in SEQ ID NO:54 or a framework region 1 having the amino acid set forth in SEQ ID NO:64. In another example, a scFv can be designed to include the six CDRs set forth in FIGS. 3A and 3B and the framework regions set forth in FIGS. 6B and 6C, FIGS. 7A and 7B, FIGS. 8A and 8B, or FIGS. 9A and 9B.

In some cases, a binder (e.g., an antibody, antigen binding fragment, antibody domain, a CAR, a cell engager, and/or an ADC) provided herein having the ability to bind to a CD94/NKG2A polypeptide (e.g., a human CD94/NKG2A polypeptide) can include (a) a heavy chain variable domain that includes an amino acid sequence having at least 90 percent identity to the amino acid sequence set forth in SEQ ID NO:24 and/or (b) a light chain variable domain that includes an amino acid sequence having at least 90 percent identity to the amino acid sequence set forth in SEQ ID NO:32. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:24 and/or (b) a light chain variable domain that includes an amino acid sequence having at least 90, 91, 92, 93, 94, 95, 96, 97, 98, or 99 percent identity to the amino acid sequence set forth in SEQ ID NO:32. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:24 and/or (b) a light chain variable domain that includes an amino acid sequence having 100 percent identity to the amino acid sequence set forth in SEQ ID NO:32.

In some cases, a binder (e.g., an antibody, antigen binding fragment, antibody domain, a CAR, a cell engager, and/or an ADC) provided herein having the ability to bind to a CD94/NKG2A polypeptide (e.g., a human CD94/NKG2A polypeptide) can include (a) a heavy chain variable domain that includes an amino acid sequence having at least 90 percent identity to the amino acid sequence set forth in SEQ ID NO:24, provided that the heavy chain variable domain includes the amino acid sequences set forth in SEQ ID NOs:17, 18, and 19, and/or (b) a light chain variable domain that includes an amino acid sequence having at least 90 percent identity to the amino acid sequence set forth in SEQ ID NO:32, provided that the light chain variable domain includes the amino acid sequences set forth in SEQ ID NOs:25, 26, and 27. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:24, provided that the heavy chain variable domain includes the amino acid sequences set forth in SEQ ID NOs:17, 18, and 19, and/or (b) a light chain variable domain that includes an amino acid sequence having at least 90, 91, 92, 93, 94, 95, 96, 97, 98, or 99 percent identity to the amino acid sequence set forth in SEQ ID NO:32, provided that the light chain variable domain includes the amino acid sequences set forth in SEQ ID NOs:25, 26, and 27.

In some cases, a binder (e.g., an antibody, antigen binding fragment, antibody domain, a CAR, a cell engager, and/or an ADC) provided herein having the ability to bind to a CD94/NKG2A polypeptide (e.g., a human CD94/NKG2A polypeptide) can include (a) a heavy chain variable domain having the amino acid sequence set forth in SEQ ID NO:24 or the amino acid set forth in SEQ ID NO:24 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:32 or the amino acid set forth in SEQ ID NO:32 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to a CD94/NKG2A polypeptide (e.g., a human CD94/NKG2A polypeptide), can include a heavy chain variable domain having the amino acid sequence set forth in SEQ ID NO:24 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:17, 18, and 19, and can include a light chain variable domain having the amino acid sequence set forth in SEQ ID NO:32 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs:25, 26, and 27.

In some cases, a binder (e.g., an antibody, antigen binding fragment, antibody domain, a CAR, a cell engager, and/or an ADC) provided herein having the ability to bind to a CD94/NKG2A polypeptide (e.g., a human CD94/NKG2A polypeptide) can include (a) a heavy chain variable domain comprising (i) a CDR1 that comprises, consists essentially of, or consists of the amino acid sequence set forth in SEQ ID NO: 17, (ii) a CDR2 that comprises, consists essentially of, or consists of the amino acid sequence set forth in SEQ ID NO:18, and (iii) a CDR3 that comprises, consists essentially of, or consists of the amino acid sequence set forth in SEQ ID NO:19, and/or (b) a light chain variable domain comprising (i) a CDR1 that comprises, consists essentially of, or consists of the amino acid sequence set forth in SEQ ID NO:25, (ii) a CDR2 that comprises, consists essentially of, or consists of the amino acid sequence set forth in SEQ ID NO:26, and (iii) a CDR3 that comprises, consists essentially of, or consists of the amino acid sequence set forth in SEQ ID NO:27. As used herein, a “CDR1 that consists essentially of the amino acid sequence set forth in SEQ ID NO:17” is a CDR1 that has zero, one, or two amino acid substitutions within SEQ ID NO:17, that has zero, one, two, three, four, or five amino acid residues directly preceding SEQ ID NO:17, and/or that has zero, one, two, three, four, or five amino acid residues directly following SEQ ID NO:17, provided that the binder (e.g., an antibody, antigen binding fragment, antibody domain, a CAR, a cell engager, and/or an ADC) maintains its basic ability to bind to a CD94/NKG2A polypeptide (e.g., a human CD94/NKG2A polypeptide). Examples of a CDR1 that consists essentially of the amino acid sequence set forth in SEQ ID NO:17 include, without limitation, those set forth in Table 7.

TABLE 7
Exemplary CDR1s that consist essentially
of the amino acid sequence set forth in
SEQ ID NO: 17.

| Sequence | SEQ ID NO: |
|---|---|
| AYYMS | 226 |
| SYYMS | 227 |
| TYYMS | 228 |
| GSYMS | 229 |
| GTYMS | 230 |
| GHYMS | 231 |
| GYSMS | 232 |
| GYTMS | 233 |
| GYHMS | 234 |
| GYYMT | 235 |

As used herein, a “CDR2 that consists essentially of the amino acid sequence set forth in SEQ ID NO: 18” is a CDR2 that has zero, one, or two amino acid substitutions within SEQ TD NO: 18, that has zero, one, two, three, four, or five amino acid residues directly preceding SEQ ID NO:18, and/or that has zero, one, two, three, four, or five amino acid residues directly following SEQ ID NO: 18, provided that the binder (e.g., an antibody, antigen binding fragment, antibody domain, a CAR, a cell engager, and/or an ADC) maintains its basic ability to bind to a CD94/NKG2A polypeptide (e.g., a human CD94/NKG2A polypeptide). Examples of a CDR2 that consists essentially of the amino acid sequence set forth in SEQ ID NO: 18 include, without limitation, those set forth in Table 8.

TABLE 8
Exemplary CDR2s that consist essentially
of the amino acid sequence set forth in
SEQ ID NO: 18.

| Sequence | SEQ ID NO: |
|---|---|
| YIYNSGRTIYYADSVKG | 236 |
| YITNSGRTIYYADSVKG | 237 |
| YIANSGRTIYYADSVKG | 238 |
| YISQSGRTIYYADSVKG | 239 |
| YISNTGRTIYYADSVKG | 240 |
| YISNYGRTIYYADSVKG | 241 |
| YISNSGKTIYYADSVKG | 242 |
| YISNSGRSIYYADSVKG | 243 |
| YISNSARTIYYADSVKG | 244 |
| YISNSSRTIYYADSVKG | 245 |

As used herein, a “CDR3 that consists essentially of the amino acid sequence set forth in SEQ ID NO: 19” is a CDR3 that has zero, one, or two amino acid substitutions within SEQ ID NO: 19, that has zero, one, two, three, four, or five amino acid residues directly preceding SEQ ID NO: 19, and/or that has zero, one, two, three, four, or five amino acid residues directly following SEQ ID NO: 19, provided that the binder (e.g., an antibody, antigen binding fragment, antibody domain, a CAR, a cell engager, and/or an ADC) maintains its basic ability to bind to a CD94/NKG2A polypeptide (e.g., a human CD94/NKG2A polypeptide). Examples of a CDR3 that consists essentially of the amino acid sequence set forth in SEQ ID NO: 19 include, without limitation, those set forth in Table 9.

TABLE 9
Exemplary CDR3s that consist essentially
of the amino acid sequence set forth in
SEQ ID NO: 19.

| Sequence | SEQ ID NO: |
|---|---|
| GWYMDA | 246 |
| GYYMDA | 247 |
| GLYMDA | 248 |
| GIYMDA | 249 |
| SFYMDA | 250 |
| AFYMDA | 251 |
| VFYMDA | 252 |
| GHYMDA | 253 |
| GWHMDA | 254 |
| GWMMDA | 255 |

As used herein, a “CDR1 that consists essentially of the amino acid sequence set forth in SEQ ID NO:25” is a CDR1 that has zero, one, or two amino acid substitutions within SEQ ID NO:25, that has zero, one, two, three, four, or five amino acid residues directly preceding SEQ ID NO:25, and/or that has zero, one, two, three, four, or five amino acid residues directly following SEQ ID NO:25, provided that the binder (e.g., an antibody, antigen binding fragment, antibody domain, a CAR, a cell engager, and/or an ADC) maintains its basic ability to bind to a CD94/NKG2A polypeptide (e.g., a human CD94/NKG2A polypeptide). Examples of a CDR1 that consists essentially of the amino acid sequence set forth in SEQ ID NO:25 include, without limitation, those set forth in Table 10.

TABLE 10
Exemplary CDR1s that consist essentially
of the amino acid sequence set forth in
SEQ ID NO: 25.

| Sequence | SEQ ID NO: |
|---|---|
| QASQSISSYLN | 196 |
| RMSQSISSYLN | 197 |
| RARQSISSYLN | 198 |
| RASESISSYLN | 199 |
| RASQGISSYLN | 200 |
| RASQSIRSYLN | 201 |
| RASQSISNYLN | 202 |
| RASQSISSWLN | 203 |
| RASQSISSYLA | 204 |
| WASQSISSYLN | 205 |

As used herein, a “CDR2 that consists essentially of the amino acid sequence set forth in SEQ ID NO:26” is a CDR2 that has zero, one, or two amino acid substitutions within SEQ ID NO:26, that has zero, one, two, three, four, or five amino acid residues directly preceding SEQ ID NO:26, and/or that has zero, one, two, three, four, or five amino acid residues directly following SEQ ID NO:26, provided that the binder (e.g., an antibody, antigen binding fragment, antibody domain, a CAR, a cell engager, and/or an ADC) maintains its basic ability to bind to a CD94/NKG2A polypeptide (e.g., a human CD94/NKG2A polypeptide). Examples of a CDR2 that consists essentially of the amino acid sequence set forth in SEQ ID NO:26 include, without limitation, those set forth in Table 11.

TABLE 11
Exemplary CDR2s that consist essentially
of the amino acid sequence set forth in
SEQ ID NO: 26.

| Sequence | SEQ ID NO: |
|---|---|
| DASSLQS | 206 |
| AAKSLQS | 207 |
| AASTLQS | 208 |
| AASSLES | 209 |
| AASSLQT | 210 |
| SASSLQS | 211 |
| AAPSLQS | 212 |
| AASNLQS | 213 |
| AASSLHS | 214 |
| AASSLQP | 215 |

As used herein, a “CDR3 that consists essentially of the amino acid sequence set forth in SEQ ID NO:27” is a CDR3 that has zero, one, or two amino acid substitutions within SEQ ID NO:27, that has zero, one, two, three, four, or five amino acid residues directly preceding SEQ ID NO:27, and/or that has zero, one, two, three, four, or five amino acid residues directly following SEQ ID NO:27, provided that the binder (e.g., an antibody, antigen binding fragment, antibody domain, a CAR, a cell engager, and/or an ADC) maintains its basic ability to bind to a CD94/NKG2A polypeptide (e.g., a human CD94/NKG2A polypeptide). Examples of a CDR3 that consists essentially of the amino acid sequence set forth in SEQ ID NO:27 include, without limitation, those set forth in Table 12.

TABLE 12
Exemplary CDR3s that consist essentially
of the amino acid sequence set forth in
SEQ ID NO: 27.

| Sequence | SEQ ID NO: |
|---|---|
| QQYGISPPFT | 216 |
| QHYGISPPFT | 217 |
| QYHGISPPFT | 218 |
| QSYGISPPFT | 219 |
| QYSGISPPFT | 220 |
| QYYAISPPFT | 221 |
| QYYSISPPFT | 222 |
| QYYGITPPFT | 223 |
| QYYTISPPFT | 224 |
| QYYGIYPPFT | 225 |

When designing a single chain antibody (e.g., a scFv) having a heavy chain variable domain and a light chain variable domain, the two regions can be directly connected or can be connected using any appropriate linker sequence. For example, a heavy chain variable domain having the CDRs of SEQ ID NOs:1-3 or SEQ ID NOs:17-19 can be directly connected to a light chain variable domain having the CDRs of SEQ ID NOs:9-11 or SEQ ID NOs:25-27, respectively, via a linker sequence. Examples of linker sequences that can be used to connect a heavy chain variable domain and a light chain variable domain to create a scFv include, without limitation, those linkers set forth in FIG. 10.

As indicated herein, the amino acid sequences described herein can include amino acid modifications (e.g., the articulated number of amino acid modifications). Such amino acid modifications can include, without limitation, amino acid substitutions, amino acid deletions, amino acid additions, and combinations. In some cases, an amino acid modification can be made to improve the binding and/or contact with an antigen and/or to improve a functional activity of a binder (e.g., an antibody, antigen binding fragment, antibody domain, a CAR, a cell engager, and/or an ADC) provided herein. In some cases, an amino acid substitution within an articulated sequence identifier can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within an articulated sequence identifier can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding the antibody or fragment thereof. See, for example, Zoller, Curr Opin. Biotechnol. 3: 348-354 (1992). Both naturally occurring and non-naturally occurring amino acids (e.g., artificially-derivatized amino acids) can be used to generate an amino acid sequence variant provided herein.

A representative number of binders (e.g., antibodies, antigen binding fragments, and/or antibody domains) having the ability to bind to a CD94/NKG2A polypeptide (e.g., a human CD94/NKG2A polypeptide) are further described in Table 13.

TABLE 13
Representative number of binders.

| | SEQ ID NOs | SEQ ID NOs of | | SEQ ID NOs | SEQ ID NOs | |
|---|---|---|---|---|---|---|
| | of Heavy | Heavy Chain | SEQ ID NO | of Light | of Light Chain | SEQ ID NO |
| | Chain | Variable | of Heavy | Chain | Variable | of Light |
| | Variable | Domain/ | Chain | Variable | Domain/ | Chain |
| Clone # | Domain/ | Region | Variable | Domain/ | Region | Variable |
| (Antibody | Region | Framework | Domain/ | Region | Framework | Domain/ |
| type) | CDRs | Regions | Region | CDRs | Regions | Region |
| #1 (Fab) | 1, 2, 3 | 4, 5, 6, 7 | 8 | 9, 10, 11 | 12, 13, 14, 15 | 16 |
| #2 (Fab) | 17, 18, 19 | 20, 21, 22, 23 | 24 | 25, 26, 27 | 28, 29, 30, 31 | 32 |

Table 14 includes an alternative designation that can be used to refer to each of Clones #1-#2.

TABLE 14
Alternative nomenclature for Clones #1-#2.

| Clone # | Alternative names |
|---|---|
| 1 | 1B2 |
| 2 | 1B2-6 |

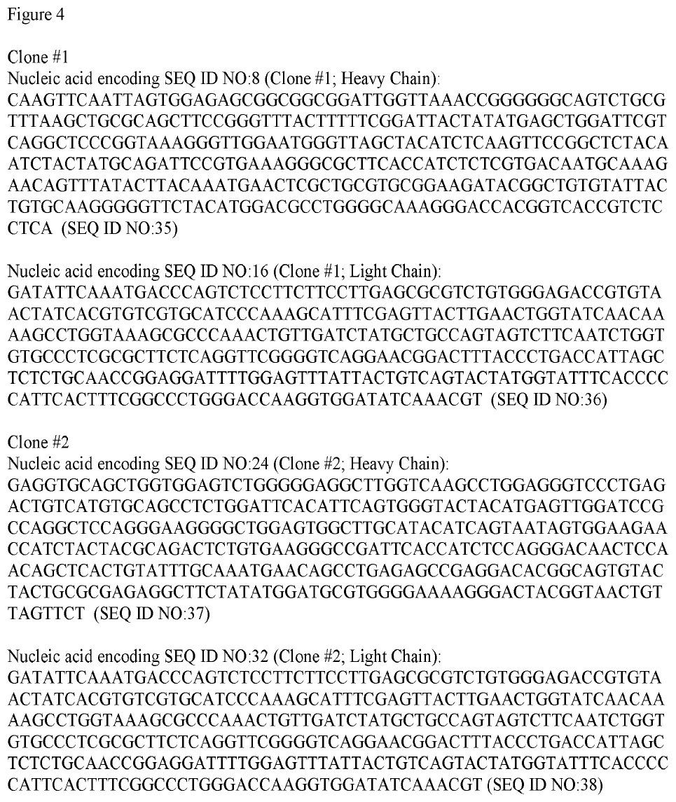

The binders (e.g., antibodies, antigen binding fragments, antibody domains, CARs, cell engagers, and/or ADCs) provided herein can be produced using any appropriate method. For example, the binders (e.g., antibodies, antigen binding fragments, antibody domains, CARs, and/or cell engagers) provided herein can be produced in recombinant host cells. For example, a nucleic acid encoding a binder (e.g., an antibody, antigen binding fragment, antibody domain, CAR, and/or cell engager) provided herein can be constructed, introduced into an expression vector, and expressed in suitable host cells. FIG. 4 is a sequence listing of nucleic acid sequences encoding exemplary binders (e.g., antibodies, antigen binding fragments, and/or antibody domains) described herein. In some cases, a binder (e.g., an antibody, antigen binding fragment, antibody domain, CAR, and/or cell engager) provided herein can be recombinantly produced in prokaryotic hosts such as E. coli, Bacillus brevis, Bacillus subtilis, Bacillus megaterium, Lactobacillus zeae/casei, or Lactobacillus paracasei. A binder (e.g., an antibody, antigen binding fragment, antibody domain, CAR, and/or cell engager) provided herein also can be recombinantly produced in eukaryotic hosts such as yeast (e.g., Pichia pastoris, Saccharomyces cerevisiae, Hansenula polymorpha, Schizosaccharomyces pombe, Schwanniomyces occidentalis, Kluyveromyces lactis, or Yarrowia lipolytica), filamentous fungi of the genera Trichoderma (e.g., T. reesei) and Aspergillus (e.g., A. niger and A. oryzae), protozoa such as Leishmania tarentolae, insect cells, or mammalian cells (e.g., mammalian cell lines such as Chinese hamster ovary (CHO) cells, Per.C6 cells, mouse myeloma NSO cells, baby hamster kidney (BHK) cells, or human embryonic kidney cell line HEK293). See, for example, the Frenzel et al. reference (Front Immunol., 4:217 (2013)).

In some cases, an antigen binding fragment or antibody domain provided herein can be produced by proteolytic digestion of an intact antibody. For example, an antigen binding fragment can be obtained by treating an antibody with an enzyme such as papain or pepsin. Papain digestion of whole antibodies can be used to produce F(ab)₂ or Fab fragments, while pepsin digestion of whole antibodies can be used to produce F(ab′)₂ or Fab′ fragments.

In some cases, a binder (e.g., an antibody, antigen binding fragment, antibody domain, CAR, cell engager, and/or ADC) provided herein can be substantially pure. The term “substantially pure” as used herein with reference to a binder (e.g., an antibody, antigen binding fragment, antibody domain, CAR, cell engager, and/or ADC) refers to the binder (e.g., an antibody, antigen binding fragment, antibody domain, CAR, cell engager, and/or ADC) as being substantially free of other polypeptides, lipids, carbohydrates, and nucleic acid with which it is naturally associated. Thus, a substantially pure binder (e.g., an antibody, antigen binding fragment, antibody domain, CAR, cell engager, and/or ADC) provided herein is any binder (e.g., an antibody, antigen binding fragment, antibody domain, CAR, cell engager, and/or ADC) that is removed from its natural environment and is at least 60 percent pure. A substantially pure binder (e.g., an antibody, antigen binding fragment, antibody domain, CAR, cell engager, and/or ADC) provided herein can be at least about 65, 70, 75, 80, 85, 90, 95, or 99 percent pure.

This document also provides bispecific binders (e.g., bispecific antibodies, bispecific antigen binding fragments, and/or bispecific antibody domains) that bind to two different epitopes with at least one being an epitope of a CD94/NKG2A polypeptide (e.g., a human CD94/NKG2A polypeptide). In some cases, a bispecific binder provided herein can be designed to bind to two different epitopes of the same CD94/NKG2A polypeptide (e.g., a human CD94/NKG2A polypeptide). In some cases, a bispecific binder provided herein can bind to a CD94/NKG2A polypeptide (e.g., a human CD94/NKG2A polypeptide) and to an epitope on a different polypeptide (e.g., a CD3 polypeptide). Bispecific binders can be produced by chemically conjugating two different binders (e.g., antibodies, antigen binding fragments, and/or antibody domains) together. Bispecific binders also can be produced by fusing two antibody-producing cells, e.g., hybridomas, to make a hybrid cell line that produces two different heavy and two different light chains within the same cell, which can result in, for example, bispecific IgG molecules. See, Brinkmann and Kontermann, MAbs., 9(2):182-212 (2017).

In some cases, a binder (e.g., an antibody, antigen binding fragment, antibody domain, CAR, and/or cell engager) provided herein can be fused or conjugated (e.g., covalently or non-covalently attached) to another polypeptide or other moiety to provide a fusion protein or conjugate. For example, a binder (e.g., an antibody, antigen binding fragment, antibody domain, CAR, and/or cell engager) provided herein can be conjugated (e.g., covalently or non-covalently attached) to a polymer (e.g., polyethylene glycol (PEG), polyethylenimine (PEI) modified with PEG (PEI-PEG), and/or polyglutamic acid (PGA) (N-(2-Hydroxypropyl) methacrylamide (HPMA) copolymers), hyaluronic acid, a fluorescent substance, a luminescent substance, a hapten, an enzyme, a metal chelate, a drug, a radioisotope, and/or a cytotoxic agent. Any appropriate method can be used to conjugate (e.g., covalently or non-covalently attach) another polypeptide or other moiety to a binder (e.g., an antibody, antigen binding fragment, antibody domain, CAR, and/or cell engager) provided herein. For example, another polypeptide or other moiety can be conjugated to a binder (e.g., an antibody, antigen binding fragment, antibody domain, CAR, and/or cell engager) provided herein using the methods described in U.S. Pat. No. 8,021,661.

In some cases, a binder (e.g., an antibody, antigen binding fragment, antibody domain, CAR, cell engager, and/or ADC) provided herein can be modified with a moiety that improves its stabilization and/or retention in circulation, for example, in blood, serum, or other tissues by, for example, at least 1.5-, 2-, 5-, 10-, or 50-fold. For example, a binder (e.g., an antibody, antigen binding fragment, antibody domain, CAR, cell engager, and/or ADC) provided herein can be attached (e.g., covalently or non-covalently attached) to a polymer such as a substantially non-antigenic polymer. Examples of substantially non-antigenic polymers that can be used as described herein include, without limitation, polyalkylene oxides and polyethylene oxides. In some cases, a polymer used herein can have any appropriate molecule weight. For example, a polymer having an average molecular weight from about 200 Daltons to about 35,000 Daltons (e.g., from about 1,000 to about 15,000 Daltons or from about 2,000 to about 12,500 Daltons) can be used. In some cases, a binder (e.g., an antibody, antigen binding fragment, antibody domain, CAR, cell engager, and/or ADC) provided herein can be attached (e.g., covalently or non-covalently) to a water soluble polymer. Examples of water soluble polymers that can be used as described herein include, without limitation, hydrophilic polyvinyl polymers, polyvinylalcohol, polyvinylpyrrolidone, polyalkylene oxide homopolymers, polyethylene glycol (PEG), polypropylene glycols, polyoxyethylenated polyols, and copolymers thereof and/or block copolymers thereof provided that the water solubility of the copolymer or block copolymers is maintained.

In some cases, a binder (e.g., an antibody, antigen binding fragment, antibody domain, CAR, cell engager, and/or ADC) provided herein can be attached (e.g., covalently or non-covalently attached) to one or more polyoxyalkylenes (e.g., polyoxyethylene, polyoxypropylene, or block copolymers of polyoxyethylene and polyoxypropylene), polymethacrylates, carbomers, branched or unbranched polysaccharides, or combinations thereof. For example, a binder (e.g., an antibody, antigen binding fragment, antibody domain, CAR, cell engager, and/or ADC) provided herein can be covalently attached to polyoxyethylene.

This document also provides ADCs. The term “ADC” as used herein refers to a conjugate that includes (a) an antigen binding domain and (b) at least one drug covalently linked directly or indirectly to that antigen binding domain. In some cases, an ADC described herein can include (a) an antigen binding domain having the ability to bind to a CD94/NKG2A polypeptide (e.g., a human CD94/NKG2A polypeptide) and (b) at least one drug covalently linked directly or indirectly to that antigen binding domain. Any appropriate binder (e.g., an antibody, antigen binding fragment, and/or antibody domain) provided herein and having the ability to bind to a CD94/NKG2A polypeptide (e.g., a human CD94/NKG2A polypeptide) can be used as an antigen binding domain to make an ADC described herein. For example, any of the binders set forth in Table 13 can be used to make an ADC having the ability to bind to a CD94/NKG2A polypeptide (e.g., a human CD94/NKG2A polypeptide). Examples of drugs that can be used to make an ADC described herein include, without limitation, IL-2, IL-12, and small molecule inhibitors of PD-L1 (e.g., BMS1166 or BMS202). In some cases, examples of drugs that can be used to make an ADC described herein include, without limitation, auristatins (e.g., monomethyl auristatin E (MMAE)), mertansine (DM-1), and pyrrolobenzodiazepine (PBD) dimers. Any appropriate ADC linker can be used to covalently attach one or more drugs to an antigen binding domain having the ability to bind to a CD94/NKG2A polypeptide (e.g., a human CD94/NKG2A polypeptide) to form an ADC provided herein. For example, cleavable or non-cleavable ADC linkers can be used to covalently attach one or more drugs to an antigen binding domain having the ability to bind to a CD94/NKG2A polypeptide (e.g., a human CD94/NKG2A polypeptide) to form an ADC provided herein. Examples of ADC linkers can be used to covalently attach one or more drugs to an antigen binding domain having the ability to bind to a CD94/NKG2A polypeptide (e.g., a human CD94/NKG2A polypeptide) to form an ADC provided herein include, without limitation, ADC disulfide linkers, ADC hydrazone linkers, ADC peptide linkers, ADC thioether linkers, and ADC PEG-containing linkers.

This document also provides nucleic acid molecules (e.g., isolated nucleic acid molecules) having a nucleic acid sequence encoding at least part of a binder (e.g., an antibody, antigen binding fragment, antibody domain, CAR, and/or cell engager) provided herein. For example, an isolated nucleic acid molecule provided herein can include a nucleic acid sequence encoding a heavy chain variable domain such as a heavy chain variable domain as set forth in FIG. 2A or 3A. In another example, an isolated nucleic acid molecule provided herein can include a nucleic acid sequence encoding a light chain variable domain such as a light chain variable domain as set forth in FIG. 2B or 3B. In some cases, an isolated nucleic acid molecule provided herein can include a nucleic acid sequence encoding both (a) a heavy chain variable domain and (b) a light chain variable domain, with or without, encoding a linker polypeptide set forth in FIG. 10. A nucleic acid provided herein (e.g., an isolated nucleic acid molecule) can be single stranded or double stranded nucleic acid of any appropriate type (e.g., DNA, RNA, or DNA/RNA hybrids).

This document also provides vectors (e.g., plasmid vectors or viral vectors) containing one or more nucleic acids provided herein. An example of a plasmid vector that can be designed to include one or more nucleic acids having a nucleic acid sequence encoding at least part of a binder (e.g., an antibody, antigen binding fragment, antibody domain, CAR, and/or cell engager) provided herein includes, without limitation, phagemids. Examples of viral vectors that can be designed to include one or more nucleic acids having a nucleic acid sequence encoding at least part of a binder (e.g., an antibody, antigen binding fragment, antibody domain, CAR, and/or cell engager) provided herein include, without limitation, retroviral vectors, parvovirus-based vectors (e.g., adenoviral-based vectors and adeno-associated virus (AAV)-based vectors), lentiviral vectors (e.g., herpes simplex (HSV)-based vectors), poxviral vectors (e.g., vaccinia virus-based vectors and fowlpox virus-based vectors), and hybrid or chimeric viral vectors. For example, a viral vector having an adenoviral backbone with lentiviral components such as those described elsewhere (Zheng et al., Nat. Biotech., 18(2): 176-80 (2000); WO 98/22143; WO 98/46778; and WO 00/17376) or viral vectors having an adenoviral backbone with AAV components such as those described elsewhere (Fisher et al., Hum. Gene Ther., 7:2079-2087 (1996)) can be designed to include one or more nucleic acids having a nucleic acid sequence encoding at least part of a binder (e.g., an antibody, antigen binding fragment, antibody domain, CAR, and/or cell engager) provided herein.

In some cases, a vector (e.g., a plasmid vector or a viral vector) provided herein can include a nucleic acid sequence encoding scFv or antibody domain (e.g., a VH domain) provided herein. In some cases, a vector (e.g., a plasmid vector or a viral vector) provided herein can include a nucleic acid sequence encoding CAR provided herein. In some cases, a vector (e.g., a plasmid vector or a viral vector) provided herein can include a nucleic acid sequence encoding cell engager provided herein.

A vector provided herein (e.g., a plasmid vector or viral vector provided herein) can include any appropriate promoter and other regulatory sequence (e.g., transcription and translation initiation and termination codons) operably linked the nucleic acid sequence encoding at least part of a binder (e.g., an antibody, antigen binding fragment, antibody domain, CAR, and/or cell engager) provided herein. In some cases, a promoter used to drive expression can be a constitutive promotor or a regulatable promotor. Examples of regulatable promoters that can be used as described herein include, without limitation, inducible promotors, repressible promotors, and tissue-specific promoters. Examples of viral promotors that can be used as described herein include, without limitation, adenoviral promotors, vaccinia virus promotors, CMV promotors (e.g., immediate early CMV promotors), and AAV promoters.

Any appropriate method can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein. For example, molecule cloning techniques can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein as described elsewhere (see, e.g., Sambrook et al., Molecular Cloning: A Laboratory Manual, 2nd edition, Cold Spring Harbor Laboratory, NY (1989); and Ausubel et al., Current Protocols in Molecular Biology, Green Publishing Associates and John Wiley & Sons, New York, N.Y. (1994)).

This document also provides host cells that include a nucleic acid provided herein (e.g., a nucleic acid having a nucleic acid sequence encoding at least part of a binder (e.g., an antibody, antigen binding fragment, antibody domain, CAR, and/or cell engager) provided herein). Host cells that can be designed to include one or more nucleic acids provided herein can be prokaryotic cells or eukaryotic cells. Examples of prokayotic cells that can be designed to include a nucleic acid provided herein include, without limitation, E. coli (e.g., Tb-1, TG-1, DH5a, XL-Blue MRF (Stratagene), SA2821, or Y1090 cells), Bacillus subtilis, Salmonella typhimurium, Serratia marcescens, or Pseudomonas (e.g., P. aerugenosa) cells. Examples of eukayotic cells that can be designed to include a nucleic acid provided herein include, without limitation, insect cells (e.g., Sf9 or Ea4 cells), yeast cells (e.g., S. cerevisiae cells), and mammalian cells (e.g., mouse, rat, hamster, monkey, or human cells). For example, VERO cells, HeLa cells, 3T3 cells, chinese hamster ovary (CHO) cells, W138 BHK cells, COS-7 cells, and MDCK cells can be designed to include a nucleic acid provided herein. Any appropriate method can be used to introduce one or more nucleic acids provided herein (e.g., a vector such as a plasmid vector or viral vector having a nucleic acid sequence encoding at least part of a binder provided herein) into a host cell. For example, calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, electroporation, or combinations thereof can be used to introduce a nucleic acid provided herein into a host cell (see, e.g., Sambrook et al., Molecular Biology: A Laboratory Manual, Cold Spring Harbor Laboratory, NY (1989); Davis et al., Basic Methods in Molecular Biology (1986); and Neumann et al., EMBO J., 1:841 (1982)).

In some cases, cells such as T cells, stem cells (e.g., induced pluripotent stem cells or mesenchymal stem cells), or NK cells can be designed to express one or more nucleic acids encoding a CAR described herein. For example, a population of T cells can be infected with viral vectors designed to express nucleic acid encoding a CAR described herein (e.g., a CAR having the ability to bind to a CD94/NKG2A polypeptide).

In some cases, cells such as T cells, stem cells (e.g., induced pluripotent stem cells or mesenchymal stem cells), or NK cells can be designed to express one or more nucleic acids encoding a cell engager described herein. For example, a population of T cells can be infected with viral vectors designed to express nucleic acid encoding a cell engager described herein (e.g., a cell engager having the ability to bind to a CD94/NKG2A polypeptide).

In some cases, a binder (e.g., an antibody, antigen binding fragment, antibody domain, CAR, and/or cell engager) provided herein can be produced using a method that includes (a) introducing nucleic acid encoding the polypeptide into a host cell; (b) culturing the host cell in culture medium under conditions sufficient to express the polypeptide; (c) harvesting the polypeptide from the cell or culture medium; and (d) purifying the polypeptide (e.g., to reach at least 50, 60, 70, 80, 90, 95, 97, 98, or 99 percent purity).

In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g. a human) having cancer to treat that mammal. In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g. a human) to reduce the number of cancer cells within the mammal and/or to increase the survival of the mammal suffering from cancer. For example, a binder (e.g., an antibody, antigen binding fragment, antibody domain, cell engager, and/or ADC) provided herein having the ability to bind to a CD94/NKG2A polypeptide (e.g., a human CD94/NKG2A polypeptide) can be formulated as a pharmaceutical composition for administration to a mammal (e.g. a human). In some cases, a pharmaceutical composition provided herein can include a pharmaceutically acceptable carrier such as a buffer, a salt, a surfactant, a sugar, a tonicity modifier, or combinations thereof as, for example, described elsewhere (Gervasi, et al., Eur. J. Pharmaceutics and Biopharmaceutics, 131:8-24 (2018)). Examples of pharmaceutically acceptable carriers that can be used to make a pharmaceutical composition provided herein include, without limitation, water, lactic acid, citric acid, sodium chloride, sodium citrate, sodium succinate, sodium phosphate, a surfactant (e.g., polysorbate 20, polysorbate 80, or poloxamer 188), dextran 40, or a sugar (e.g., sorbitol, mannitol, sucrose, dextrose, or trehalose), or combinations thereof. For example, a pharmaceutical composition designed to include a binder (e.g., an antibody, antigen binding fragment, antibody domain, CAR, cell engager, and/or ADC) provided herein (or a nucleic acid, a vector, or a host cell provided herein) can be formulated to include a buffer (e.g., an acetate, citrate, histidine, succinate, phosphate, or hydroxymethylaminomethane (Tris) buffer), a surfactant (e.g., polysorbate 20, polysorbate 80, or poloxamer 188), and a sugar such as sucrose. Other ingredients that can be included within a pharmaceutical composition provided herein include, without limitation, amino acids such as glycine or arginine, antioxidants such as ascorbic acid, methionine, or ethylenediaminetetraacetic acid (EDTA), anticancer agents such as enzalutamide, imanitib, gefitinib, erlotini, sunitinib, lapatinib, nilotinib, sorafenib, temsirolimus, everolimus, pazopanib, crizotinib, ruxolitinib, axitinib, bosutinib, cabozantinib, ponatinib, regorafenib, ibrutinib, trametinib, perifosine, bortezomib, carfilzomib, batimastat, ganetespib, obatoclax, navitoclax, taxol, paclitaxel, or bevacizumab, or combinations thereof. For example, a pharmaceutical composition provided herein can be formulated to include one or more binders (e.g., one or more antibodies, one or more antigen binding fragments, one or more antibody domains, one or more cells designed to express a CAR having the ability to bind to a CD94/NKG2A polypeptide, one or more cell engagers, and/or one or more ADCs) provided herein in combination with one or more checkpoint inhibitors such as anti-PD-1 antibodies or PD-1 inhibitors (e.g., cemiplimab, nivolumab, pembrolizumab, JTX-4014, spartalizumab, camrelizumab, sintilimab, tislelizumab, toripalimab, dostarlimab, INCMGA00012, AMP-224, or AMP-514), anti-PD-L1 antibodies or PD-L1 inhibitors (e.g., avelumab, durvalumab, atezolizumab, KN035, CK-301, AUNP12, CA-170, or BMS-986189), and/or anti-CTLA-4 antibodies (e.g., ipilimumab).

In some cases, when a pharmaceutical composition is formulated to include one or more binders (e.g., one or more antibodies, one or more antigen binding fragments, one or more antibody domains, one or more cells designed to express a CAR having the ability to bind to a CD94/NKG2A polypeptide, one or more cell engagers, and/or one or more ADCs) provided herein, any appropriate concentration of the binder can be used. For example, a pharmaceutical composition provided herein can be formulated to be a liquid that includes from about 1 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, CAR⁺ cell population, cell engager, and/or ADC) provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, cell engager, and/or ADC) provided herein. In some cases, a pharmaceutical composition containing a binder (e.g., an antibody, antigen binding fragment, and/or antibody domain) provided herein can be formulated as a dosage form with a titer of the binder being from about 1×10⁵ to about 1×10¹² (e.g., from about 1×10⁵ to about 1×10¹⁰, from about 1×10⁵ to about 1×10⁸, from about 1×10⁶ to about 1×10¹², from about 1×10⁶ to about 1×10¹², from about 1×10⁸ to about 1×10¹², from about 1×10⁹ to about 1×10¹², from about 1×10⁶ to about 1×10¹¹, or from about 1×10⁷ to about 1×10¹⁰).

In some cases, when a pharmaceutical composition is formulated to include one or more nucleic acids (e.g., vectors such as viral vectors) encoding at least part of a binder (e.g., an antibody, antigen binding fragment, antibody domain, CAR, and/or cell engager) provided herein, any appropriate concentration of the nucleic acid can be used. For example, a pharmaceutical composition provided herein can be formulated to be a liquid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a nucleic acid provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a nucleic acid provided herein.

In some cases, a pharmaceutical composition designed to include a binder (e.g., an antibody, antigen binding fragment, antibody domain, cell engager, and/or ADC) provided herein can be formulated to include one or more agents capable of reducing aggregation of the binder when formulated. Examples of such agents that can be used as described herein include, without limitation, methionine, arginine, lysine, aspartic acid, glycine, glutamic acid, and combinations thereof. In some cases, one or more of these amino acids can be included within the formulation at a concentration from about 0.5 mM to about 145 mM (e.g., from about 1 mM to about 145 mM, from about 10 mM to about 145 mM, from about 100 mM to about 145 mM, from about 0.5 mM to about 125 mM, from about 0.5 mM to about 100 mM, from about 0.5 mM to about 75 mM, or from about 10 mM to about 100 mM).

A pharmaceutical composition provided herein can be in any appropriate form. For example, a pharmaceutical composition provided herein can designed to be a liquid, a semi-solid, or a solid. In some cases, a pharmaceutical composition provided herein can be a liquid solution (e.g., an injectable and/or infusible solution), a dispersion, a suspension, a tablet, a pill, a powder, a microemulsion, a liposome, or a suppository. In some cases, a pharmaceutical composition provided herein can be lyophilized. In some cases, a pharmaceutical composition provided herein (e.g., a pharmaceutical composition that includes one or more binders (e.g., one or more antibodies, one or more antigen binding fragments, one or more antibody domains, one or more cell engagers, and/or one or more ADCs) provided herein can be formulated with a carrier or coating designed to protect against rapid release. For example, a pharmaceutical composition provided herein can be formulated as a controlled release formulation or as a regulated release formulation as described elsewhere (U.S. Patent Application Publication Nos. 2019/0241667; 2019/0233522; and 2019/0233498).

This document also provides methods for administering a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) to a mammal (e.g., a human). For example, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g., a human) having cancer to treat that mammal. In some cases, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g. a human) to reduce the number of cancer cells within the mammal and/or to increase the survival of the mammal suffering from cancer.

Any appropriate cancer can be treated using a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein). For example, a mammal (e.g., a human) having cancer can be treated by administering a composition (e.g., a pharmaceutical composition) containing one or more binders (e.g., one or more antibodies, one or more antigen binding fragments, one or more antibody domains, one or more cell engagers, and/or one or more ADCs) provided herein to that mammal. Examples of cancers that can be treated as described herein include, without limitation, lung cancer, prostate cancer, esophageal cancer, stomach cancer, colorectal cancer, liver cancer, vaginal cancer, cervical cancer, pancreatic cancer, head and neck cancer, blood cancer, skin cancer, brain cancer, bone cancer, and breast cancer. In some cases, a mammal (e.g., a human) having a cancer (e.g., a lung cancer, a prostate cancer, an esophageal cancer, a stomach cancer, a colorectal cancer, a liver cancer, a vaginal cancer, or a cervical cancer) can be administered a composition (e.g., a pharmaceutical composition) containing one or more binders (e.g., one or more antibodies, one or more antigen binding fragments, one or more antibody domains, one or more cell engagers, and/or one or more ADCs) provided herein to treat that mammal (e.g., to reduce the number of cancer cells within the mammal).

Any appropriate viral infection can be treated using a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein). For example, a mammal (e.g., a human) having an HIV viral infection, a hepatitis viral infection, a herpes viral infection, an influenza viral infection, a coronavirus infection, a cytomegalovirus infection, or a hendra viral infection can be treated by administering a composition (e.g., a pharmaceutical composition) containing one or more binders (e.g., one or more antibodies, one or more antigen binding fragments, one or more antibody domains, one or more cell engagers, and/or one or more ADCs) provided herein to that mammal.

Any appropriate method can be used to administer a composition (e.g., a pharmaceutical composition) provided herein to a mammal (e.g., a human). For example, a composition provided herein (e.g., a pharmaceutical composition containing one or more binders provided herein such as one or more antibodies, one or more antigen binding fragments, one or more antibody domains, one or more cell engagers, and/or one or more ADCs provided herein) can be administered to a mammal (e.g., a human) intravenously (e.g., via an intravenous injection or infusion), subcutaneously (e.g., via a subcutaneous injection), intraperitoneally (e.g., via an intraperitoneal injection), orally, via inhalation, or intramuscularly (e.g., via intramuscular injection). In some cases, the route and/or mode of administration of a composition (e.g., a pharmaceutical composition provided herein) can be adjusted for the mammal being treated.

In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that reduces the number of cancer cells and/or virally infected cells within a mammal having cancer without producing significant toxicity to the mammal. In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that increases the survival time of a mammal having cancer (and/or a viral infection) as compared to a control mammal having comparable cancer (and/or a viral infection) and not treated with the composition. For example, an effective amount of a binder (e.g., an antibody, antigen binding fragment, antibody domain, cell engager, and/or ADC) provided herein can be from about 0.001 mg/kg to about 100 mg/kg (e.g., from about 0.001 mg/kg to about 90 mg/kg, from about 0.001 mg/kg to about 80 mg/kg, from about 0.001 mg/kg to about 70 mg/kg, from about 0.001 mg/kg to about 60 mg/kg, from about 0.001 mg/kg to about 50 mg/kg, from about 0.001 mg/kg to about 40 mg/kg, from about 0.001 mg/kg to about 30 mg/kg, from about 0.005 mg/kg to about 100 mg/kg, from about 0.01 mg/kg to about 100 mg/kg, from about 0.05 mg/kg to about 100 mg/kg, from about 0.1 mg/kg to about 100 mg/kg, from about 0.5 mg/kg to about 100 mg/kg, from about 1 mg/kg to about 100 mg/kg, from about 5 mg/kg to about 100 mg/kg, from about 0.01 mg/kg to about 25 mg/kg, from about 0.1 mg/kg to about 30 mg/kg, from about 0.15 mg/kg to about 25 mg/kg, from about 0.2 mg/kg to about 20 mg/kg, from about 0.5 mg/kg to about 20 mg/kg, from about 1 mg/kg to about 30 mg/kg, from about 1 mg/kg to about 25 mg/kg, from about 1 mg/kg to about 20 mg/kg, from about 2 mg/kg to about 20 mg/kg, from about 5 mg/kg to about 30 mg/kg, from about 10 mg/kg to about 30 mg/kg, from about 15 mg/kg to about 30 mg/kg, from about 20 mg/kg to about 30 mg/kg, from about 3 mg/kg to about 30 mg/kg, from about 0.5 mg/kg to about 10 mg/kg, from about 1 mg/kg to about 10 mg/kg, from about 1 mg/kg to about 5 mg/kg, or from about 1 mg/kg to about 3 mg/kg). The effective amount can remain constant or can be adjusted as a sliding scale or variable dose depending on the mammal's response to treatment. Various factors can influence the actual effective amount used for a particular application. For example, the severity of cancer when treating a mammal having cancer, the route of administration, the severity of a viral infection when treating a mammal having a viral infection, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective amount of a composition provided herein (e.g., a pharmaceutical composition containing one or more binders provided herein) that is administered.

In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that reduces the number of cancer cells (and/or virally infected cells) within a mammal having cancer (and/or a viral infection) without producing significant toxicity to the mammal. In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that increases the survival time of a mammal having cancer (and/or a viral infection) as compared to a control mammal having comparable cancer (and/or a viral infection) and not treated with the composition. For example, an effective frequency of administration of a pharmaceutical composition provided herein such as a pharmaceutical composition containing one or more binders provided herein can be from about twice daily to about once a year (e.g., from about twice daily to about once a month, from about twice daily to about once a week, from about once daily to about once a month, or from one once daily to about once a week). In some cases, the frequency of administration of a pharmaceutical composition provided herein such as a pharmaceutical composition containing one or more binders provided herein can be daily. The frequency of administration of a pharmaceutical composition provided herein such as a pharmaceutical composition containing one or more binders provided herein can remain constant or can be variable during the duration of treatment. Various factors can influence the actual effective frequency used for a particular application. For example, the severity of a cancer, the severity of a viral infection, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective frequency of administration of a composition provided herein (e.g., a pharmaceutical composition containing one or more binders provided herein).

In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that reduces the number of cancer cells (and/or virally infected cells), within a mammal without producing significant toxicity to the mammal. In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that increases the survival time of a mammal having cancer (and/or a viral infection) as compared to a control mammal having comparable cancer (and/or a viral infection) and not treated with the composition. For example, an effective duration of administration of a pharmaceutical composition provided herein such as a pharmaceutical composition containing one or more binders provided herein can vary from a single time point of administration to several weeks to several months (e.g., 4 to 12 weeks). Multiple factors can influence the actual effective duration used for a particular application. For example, the severity of a cancer, the severity of a viral infection, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective duration of administration of a composition provided herein (e.g., a pharmaceutical composition containing one or more binders provided herein).

In some cases, a binder (e.g., an antibody, antigen binding fragment, and/or antibody domain) provided herein can be used to detect the presence or absence of a CD94/NKG2A polypeptide (e.g., a human CD94/NKG2A polypeptide) in vitro, in situ, or in vivo (e.g., in vivo imaging within a mammal such as a human). For example, a binder (e.g., an antibody, antigen binding fragment, and/or antibody domain) provided herein can be designed to include a label (e.g., a covalently attached radioactive, enzymatic, colorimetric, or fluorescent label). The labelled binder can be used to detect the presence or absence of a CD94/NKG2A polypeptide (e.g., a human CD94/NKG2A polypeptide) within a biological sample in vitro. Examples of biological samples that can be assessed using a binder (e.g., an antibody, antigen binding fragment, and/or antibody domain) provided herein include, without limitation, serum samples, plasma samples, tissue samples, biopsy samples, cell line samples, and tissue culture samples. In some cases, a biological sample that can be assessed as described herein can include mammalian body tissues and/or cells such as leukocytes, T cells, NK cells, ovary tissue or cells, prostate tissue or cells, heart tissue or cells, placenta tissue or cells, pancreas tissue or cells, liver tissue or cells, spleen tissue or cells, lung tissue or cells, breast tissue or cells, head and neck tissue or cells, endometrium tissue or cells, colon tissue or cells, colorectal tissue or cells, cervix tissue or cells, stomach tissue or cells, or umbilical tissue or cells that may express a CD94/NKG2A polypeptide (e.g., a human CD94/NKG2A polypeptide). In some cases, a binder (e.g., an antibody, antigen binding fragment, and/or antibody domain) provided herein can be immobilized, e.g., on a support, and retention of a CD94/NKG2A polypeptide (e.g., a human CD94/NKG2A polypeptide) from a biological sample on the support can be detected, and/or vice versa. In some cases, a binder (e.g., an antibody, antigen binding fragment, and/or antibody domain) provided herein can be used in applications such as fluorescence polarization, microscopy, ELISA, centrifugation, chromatography, and/or cell sorting (e.g., fluorescence activated cell sorting).

In some cases, a binder (e.g., an antibody, antigen binding fragment, and/or antibody domain) provided herein containing a label (e.g., a covalently attached radioactive label) can be used to detect the presence or absence of a CD94/NKG2A polypeptide (e.g., a human CD94/NKG2A polypeptide) within a mammal (e.g., a human). For example, a binder (e.g., an antibody, antigen binding fragment, and/or antibody domain) provided herein that is labelled (e.g., covalently labelled) with a radiolabel or an MRI detectable label can be administered to a mammal (e.g., a human), and that mammal can be assessed using a means for detecting the detectable label. In some cases, a mammal can be scanned to evaluate the location(s) of a labelled binder provided herein within the mammal. For example, the mammal can be imaged using NMR or other tomographic techniques.

Examples of labels that can be attached (e.g., covalently or non-covalently attached) to a binder (e.g., an antibody, antigen binding fragment, and/or antibody domain) provided herein include, without limitation, radiolabels such as ¹³¹I, ¹¹¹In, ¹²³I, ^{99m}Tc, ³²P, ³³P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as a peroxidase or a phosphatase. In some cases, short-range radiation emitters such as isotopes detectable by short-range detector probes can be used.

The invention will be further described in the following examples, which do not limit the scope of the invention described in the claims.

### EXAMPLES

### Example 1—Obtaining Binders Having the Ability to Bind to a Human CD94/NKG2A Polypeptide

A vector was generated to express a CD94/NKG2A polypeptide having amino acid residues 57 to 179 of CD94 and 113 to 233 of NKG2A fused to human IgG1 Fc. Between CD94 and NKG2A, the GSGGSGG (SEQ ID NO:258) linker was used (FIG. 1). This CD94/NKG2A-IgG1 fusion polypeptide was used to identify one Fab antibody fragment (Clone #1; FIGS. 2A-B). To identify this Fab, the CD94/NKG2A-IgG1 fusion polypeptide was applied to three rounds of a panning process.

A phage library was pre-blocked with 3% bovine serum albumin (BSA) in PBS (w/v) for 1 hour at 25° C. Blocked phages incubated with 10 nM biotinylated single chain CD94/NKG2A-Fc for 1 hour at 25° C. in the presence of 50 nM of single chain CD94/NKG2C-Fc. Bound phages were separated by streptavidin coated magnetic beads and washed 10 times with 1 mL of PBS pH 7.4 containing 0.1% Tween-20 (w/v). Elution of bound phages was conducted by adding either 1 μM anti-NKG2A antibody or 5 nM HLA-E (HLA-E*01:01 HLA-A leader 3-11 Monomer-VMAPRTLVL (SEQ ID NO: 325)) tetramer. For 2nd and 3rd rounds of panning, reduced concentration of biotinylated single chain CD94/NKG2A (5 nM and 1 nM, respectively) and increased competitor ratio of single chain CD94/NKG2C were applied. After 3 rounds of panning, individual clones were analyzed in ELISA and then selected clones were sequenced after plasmid rescue.

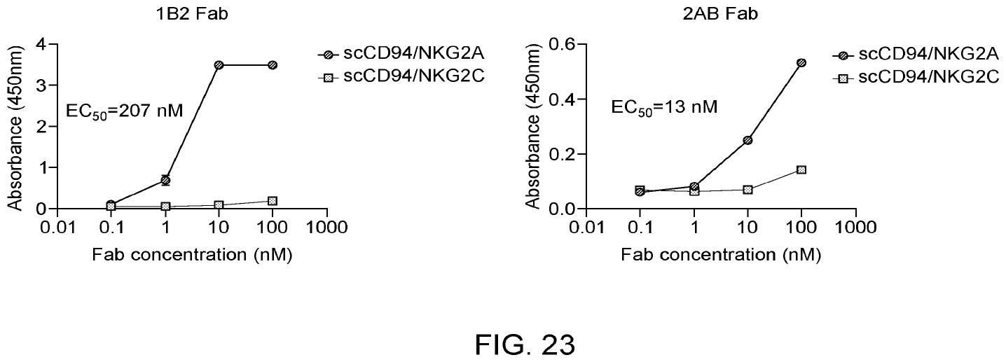

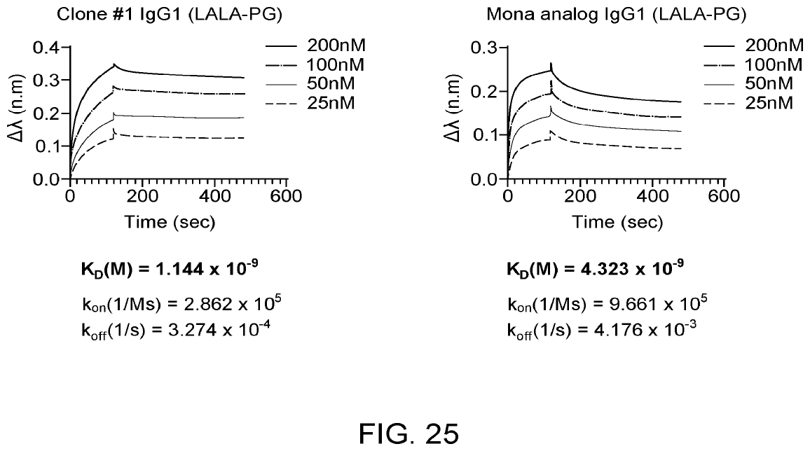

The Fab Clone #1 bound CD94/NKG2A polypeptides with high affinity and specificity. The Fab Clone #1 showed 2.7 nM EC₅₀ value in ELISA against CD94/NKG2A, while Clone #1 did not cross react with a CD94/NKG2C polypeptide in an ELISA experiment (FIG. 23). IgG Clone #1 showed 1.1 nM K_{D} (equilibrium dissociation constant) in BLItz biolayer interferometer (FIG. 25). Moreover, IgG Clone #1 competed with both tetrameric HLA-E for binding to CD94/NKG2A polypeptides, while it did not affect to binding against CD94/NKG2C (FIG. 26). These results demonstrate that Clone #1 is specific to NKG2A, not to NKG2C, and it binds to the HLA-E binding interface of NKG2A resulting in Clone #1 blocking inhibitory interactions between NKG2A and HLA-E (or potentially other HLA molecules or receptors that bind to a similar interface of NKG2A for HLA-E).

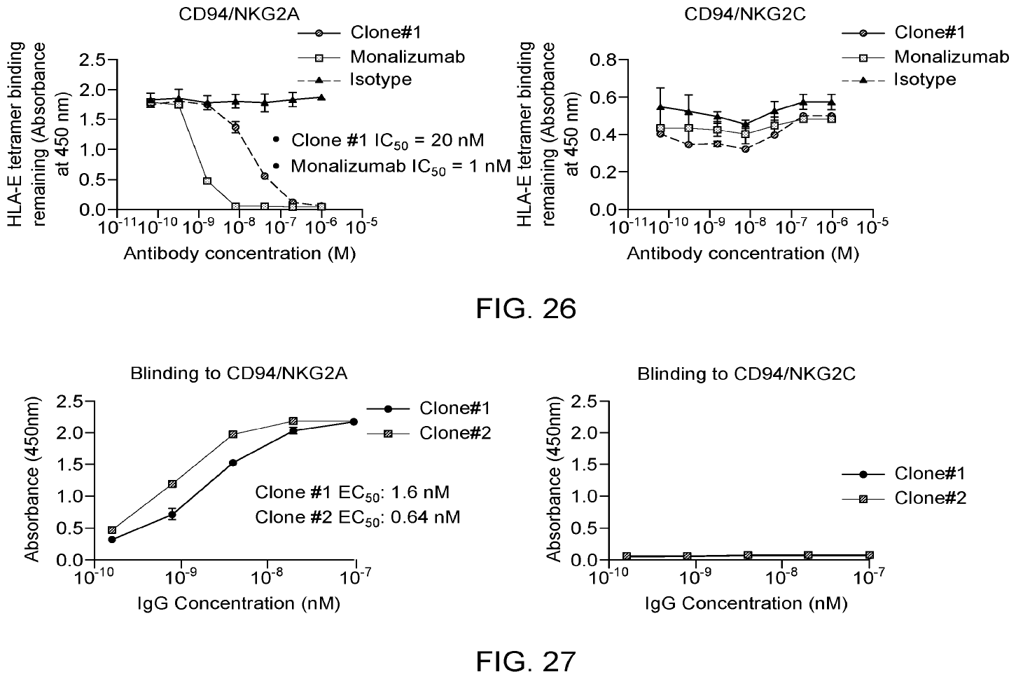

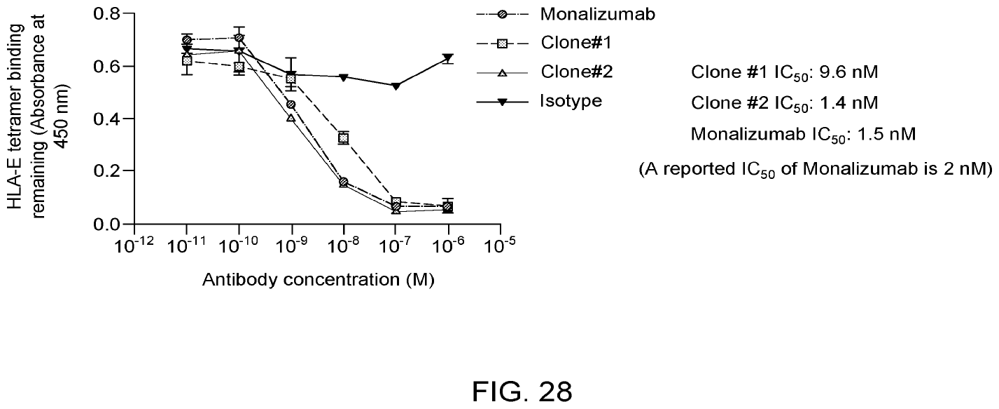

To improve binding affinity of Clone #1, yeast display technology and light chain shuffling strategy were applied to generate Clone #2. The IgG Clone #2 exhibited improved binding in ELISA compared to Clone #1, and the EC₅₀ was measured as 0.6 nM (FIG. 27). Clone #2 also did not show cross-reactivity to NKG2C. These results demonstrate that Clone #2 retains the specificity to NKG2A. IgG Clone #2 also exhibited improved competition for binding to CD94/NKG2A polypeptides compared to the original Clone #1, which was comparable to that of monalizumab (FIG. 28).

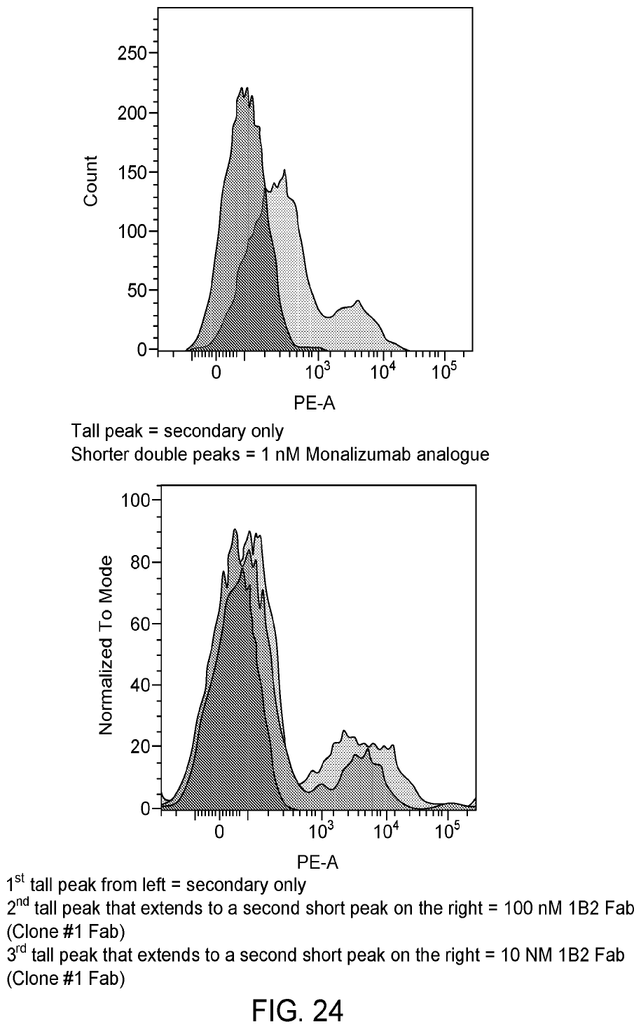

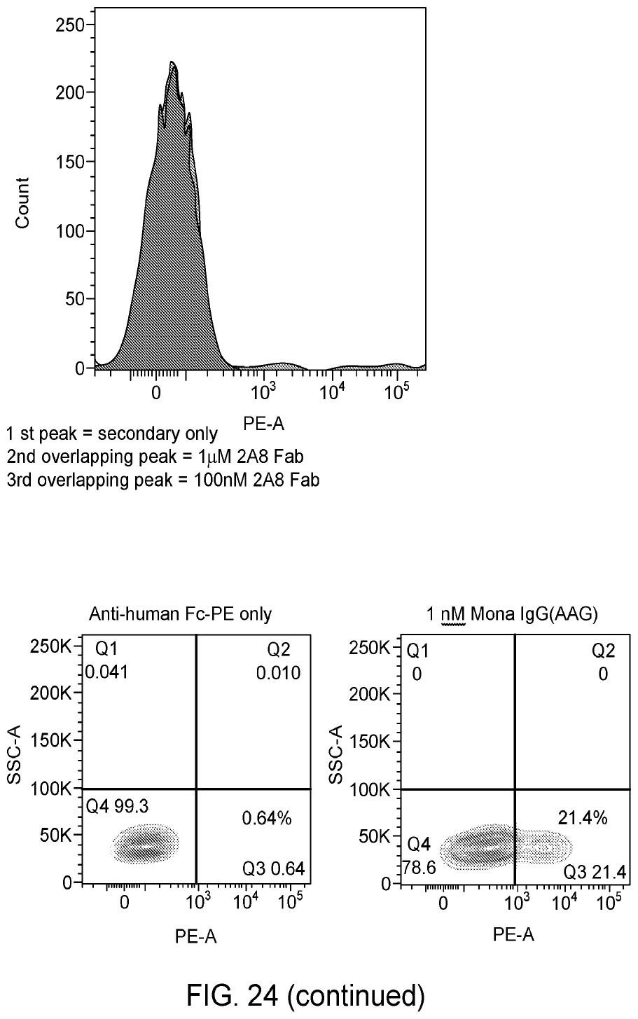

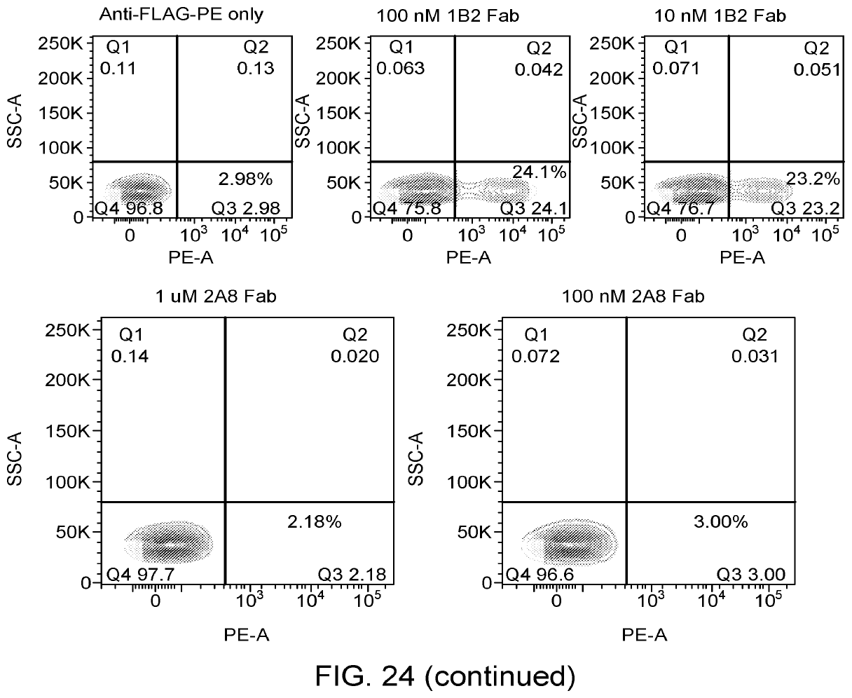

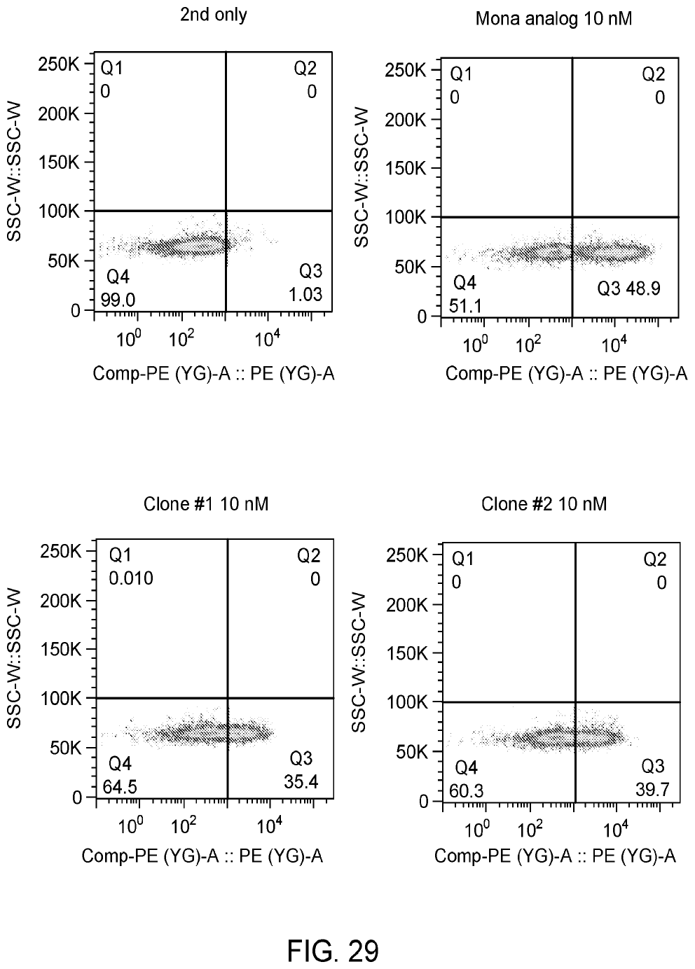

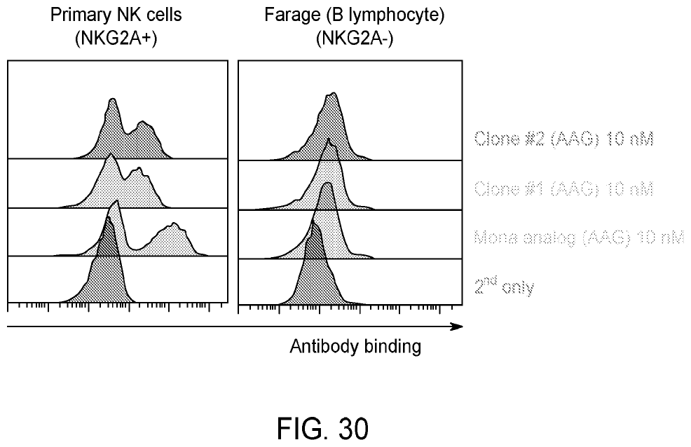

To confirm the cell surface NKG2A binding of antibodies (monalizumab analog, Clone #1, and Clone #2), primary NK cells were enriched from normal human peripheral blood mononuclear cells (PBMCs) using NK cell isolation kit. Primary NK cells were treated with antibodies (10 nM) for 1 hour at 4° C. and then stained using an Alexa647-conjugated goat anti-human IgG for 0.5 hour at 4° C. Staining was detected for the monalizumab analog, Clone #1, and Clone #2, thereby indicating that all three antibodies bound to intact NKG2A receptor on the surface of NK cell as well as to recombinant NKG2A (FIGS. 24, 29, and 30).

### Example 2—Functional Assay with Binders Having the Ability to Bind to a Human CD94/NKG2A Polypeptide

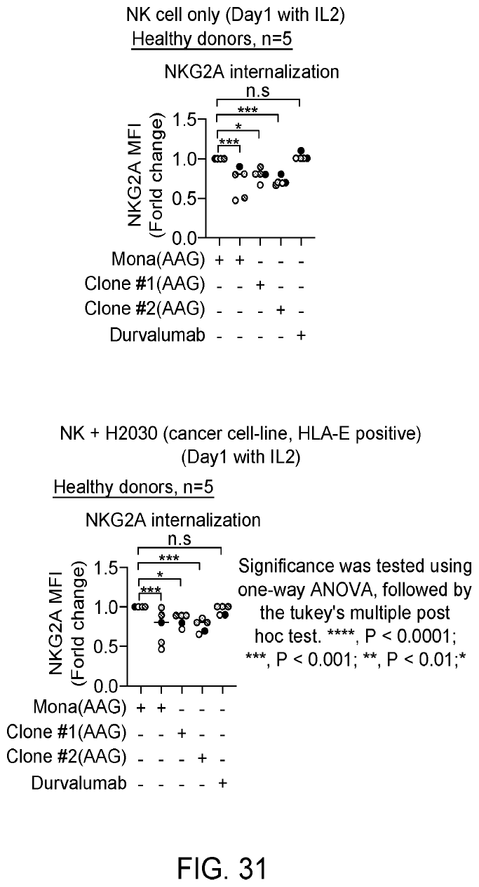

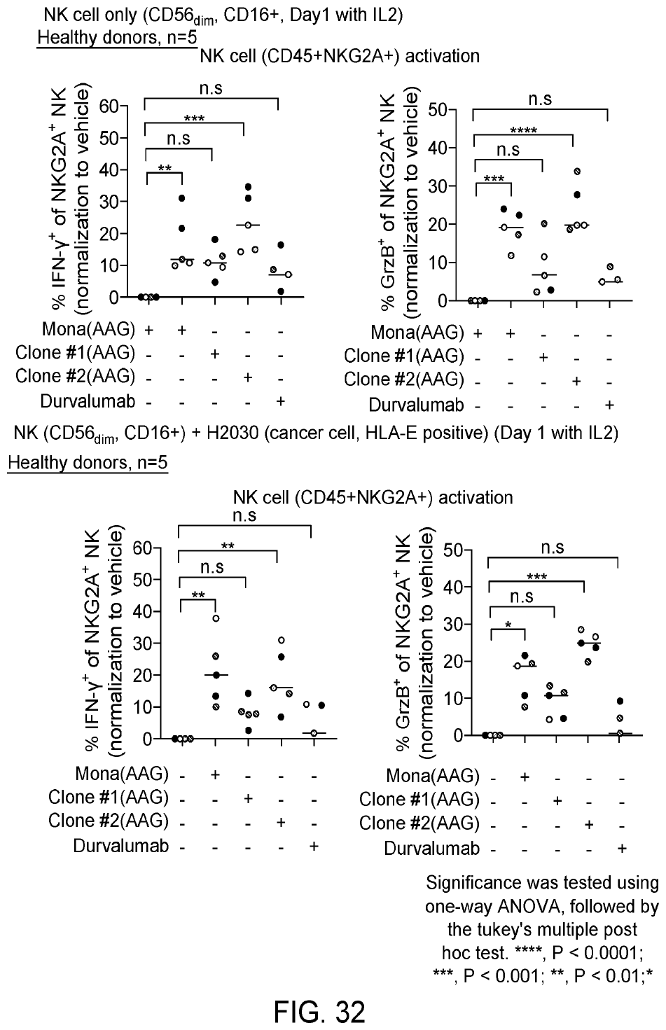

To conduct functional assays for Clone #1 and #2, purified human primary NK cells from PBMCs were used as effector cells. NK cells were activated with 50 IU/mL hIL-2 and treated with the antibodies (monalizumab analog, Clone #1, and Clone #2) for 24 hours at 37° C. in absence or presence of HLA-E-positive H2030 cells (E:T ratio of 5:1). First, changes of NKG2A expression levels of NK cells were investigated. Antibody-treated NK cells were washed with cold PBS, and then the cell surface NKG2A was detected using FITC- or PE-conjugated anti-human CD159a (NKG2A) antibody. For gating of NK cells in the co-culture system with H2030 cells, APC conjugated anti-human CD45 antibody was used. Treatment with the antibodies induced NKG2A down-regulation on the cell surface, which was potentially caused by receptor internalization (FIG. 31). Next, to examine NK cell activation, IFNγ and granzyme B were analyzed by intracellular staining. The GolgiPlug containing brefeldin A was added for the final 4 hours of culture to inhibit protein transport from the endoplasmic reticulum to the Golgi apparatus. All intracellular staining was performed using a BD cytofix/cytoperm kit. After treatment with antibodies, cells were fixed and intracellularly stained with a PE-conjugated anti-human IFNγ antibody and a PE-conjugated anti-human granzyme B antibody. Throughout all flow-cytometry experiments, labeled cells were washed twice and analyzed on the BD LSR II flow cytometry. Observed flow cytometry results demonstrated that treatment with Clone #2 or the monalizumab analog activated NK cells and increased secretion of IFNγ and granzyme B with or without co-culture with H2030 cell lines, while treatment with Clone #1 and durvalumab (an anti-PD-L1 antibody) did not show activation of NK cells (FIG. 32). Although Clone #1 did not exhibit statistical significance, the activating tendency was observed, compared to durvalumab. These results demonstrate that targeting NKG2A activates NK cells through blocking of interaction to HLA-E and/or decreasing surface expression level of NKG2A, and also binding affinity is involved in the activation of NK cells.

### Example 3—Designing Cell Engagers from Binders Having the Ability to Bind to a Human CD94/NKG2A Polypeptide

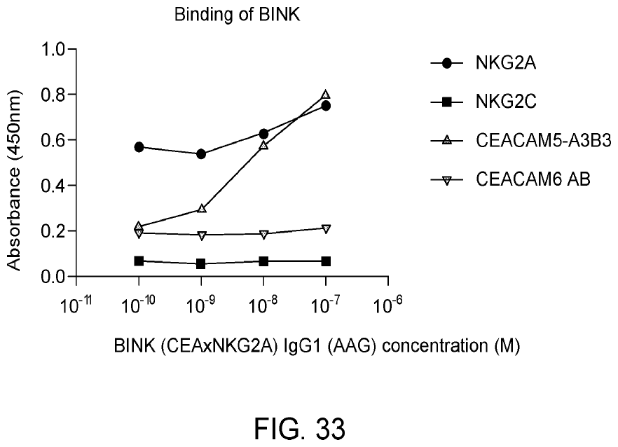

The following was performed to demonstrate the design of a bispecific NK cell/T cell engager (BiKE/BiTE, also referred to as a BINK herein) having the ability to bind to a CD94/NKG2A polypeptide on NK cells or T cells and a tumor associated antigen (TAA) on cancer cells. The CEACAM5 (CD66e) polypeptide was chosen as an example of TAA. The anti-CEACAM5 scFv was fused to the N-terminus of the Clone #1 or Clone #2 light chain via a GSGGGS (SEQ ID NO:318) linker (see, e.g., FIGS. 17A and 17B). Plasmid DNA encoding the cloned BINK was transfected to HEK239F cells and expressed for 5-7 days post-transfection. Expressed BINK polypeptides were purified by affinity chromatography with a protein A resin. Elution of bound BINK polypeptides was performed by adding 50 mM glycine buffer pH 3.0, and then storage buffer was changed to phospho-buffered saline pH 7.4 (PBS) by PD-10 desalting column. Protein purity was estimated in either SDS-PAGE or size exclusion chromatography packed with Superdex 200 increase 10/300 GL. The concentration of each BINK polypeptide was determined by Nano Drop spectrophotometer 2000C. Binding and specificity of each BINK polypeptide to a single chain CD94/NKG2A polypeptide, a single chain CD94/NKG2C polypeptide, a CEACAM5 A3B3 domain, or a CEACAM6 AB domain were analyzed through indirect ELISA. Briefly, Fc-fused proteins were coated on a 96 well plate at 200 ng/well (50 μL volume) in PBS for 2 hours at 25° C. Blocking was carried out with 3% skim-milk in PBS for overnight at 4° C. The next day, various concentrations of the BINK polypeptides were added to antigen coated plates and incubated for 1 hour at 25° C. After washing three times with PBS-T (PBS pH 7.4 containing 0.1% Tween-20), an HRP-conjugated goat anti-human kappa antibody (1:3000 dilution) was added to detect binding of the BINK polypeptide and incubated for 1 hour at 25° C. After washing three times with PBS-T, 50 μL of TMB (Thermo, PI34028) was added as a substrate, and the enzymatic reaction was stopped by adding 2N sulfonic acid. The BINK molecule exhibited binding specificity to NKG2A and CECAM5-A3B3 as designed (FIG. 33).

### Example 4—Designing Additional Cell Engagers from Binders Having the Ability to Bind to a Human CD94/NKG2A Polypeptide

The following was performed to demonstrate the design of additional bispecific NK cell/T cell engagers (BiKEs/BiTEs, also referred to as BiNKs herein) having the ability to bind to a CD94/NKG2A polypeptide on NK cells or T cells and a tumor associated antigen (TAA) on cancer cells. In one example, a HER2 polypeptide was chosen as an example of TAA. In another example, a EGFR polypeptide was chosen as an example of TAA.

An anti-HER2 scFv was fused to the N-terminus of Clone #1 light chain via a GSGGGS (SEQ ID NO:318) linker to create a BiNK referred to as “BiNK (anti-HER2×anti-NKG2A)” (see, e.g., FIGS. 17A and 17C). An anti-EGFR scFv was fused to the N-terminus of Clone #1 light chain via a GSGGGS (SEQ ID NO:318) linker to create a referred to as “BiNK (anti-EGFR×anti-NKG2A)” (see, e.g., FIGS. 17A and 17D). Briefly, plasmid DNA encoding the cloned BiNK was transfected to HEK239F cells and expressed for 5-7 days post-transfection. Expressed BiNK polypeptides were purified, and binding of each BiNK polypeptide to a single chain CD94/NKG2A polypeptide, a single chain CD94/NKG2C polypeptide, a HER2 polypeptide, or an EGFR polypeptide were analyzed through indirect ELISA.

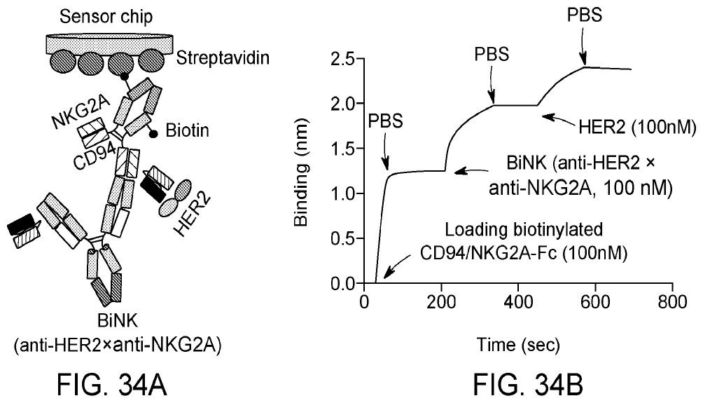

BiNK (anti-HER2×anti-NKG2A) bound to single chain CD94/NKG2A polypeptide and HER2 (FIGS. 34A and 34B) and did not bind to single chain CD94/NKG2C polypeptide. Flow cytometry also confirmed that BiNK (anti-EGFR×anti-NKG2A) bound to single chain CD94/NKG2A polypeptide and EGFR and did not bind to single chain CD94/NKG2C polypeptide.

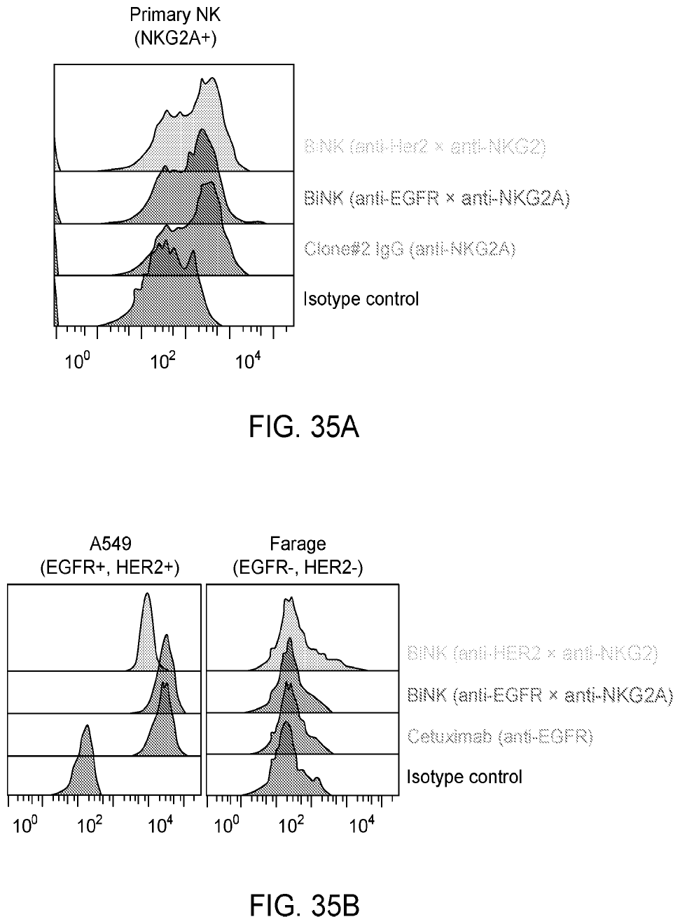

Primary NK cells express CD94/NKG2A polypeptides, and both BiNK (anti-HER2×anti-NKG2A) and BiNK (anti-EGFR×anti-NKG2A) as well as Clone #2 in the IgG format exhibited binding to primary NK cells (FIG. 35A). A549 cells express both HER2 and EGFR polypeptides, and both BiNK (anti-HER2×anti-NKG2A) and BiNK (anti-EGFR×anti-NKG2A) as well as Cetuximab exhibited binding to A549 cells (FIG. 35B). Farage cells do not express HER2, EGFR, or NKG2A polypeptides, and none of BiNK (anti-HER2×anti-NKG2A), BiNK (anti-EGFR×anti-NKG2A), and Cetuximab exhibited appreciable binding to Farage cells (FIG. 35B).

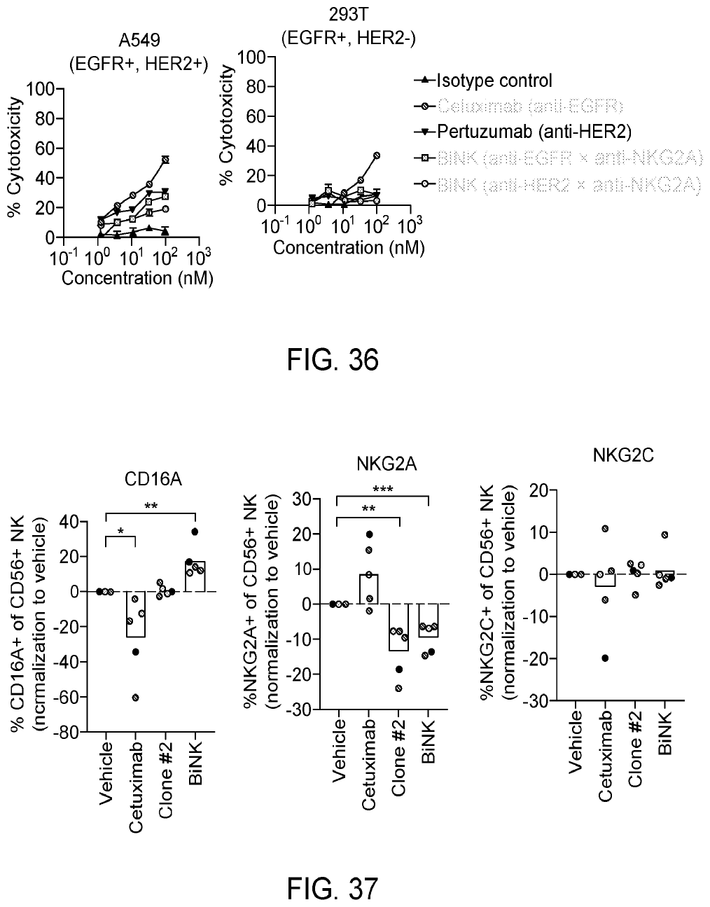

When assessing cell killing by NK cells in the presence of different binders, BiNK (anti-HER2×anti-NKG2A), BiNK (anti-EGFR×anti-NKG2A), Cetuximab, and Pertuzumab promoted the killing of A549 cells (FIG. 36).

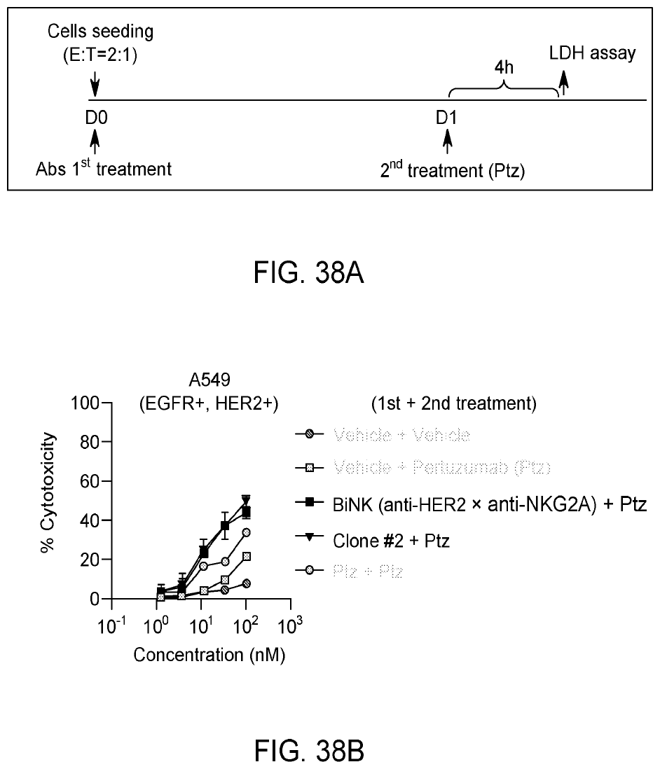

In the co-incubation culture system of primary NK cells and A549 cells, BiNK (anti-EGFR×anti-NKG2A) treatment increased the CD16A⁺ NK cell population (%) of CD56⁺ NK cells and decreased NKG2A⁺ cell population (%) of CD56⁺ NK cells, but did not change the NKG2C population, compared to the vehicle treated group (FIG. 37). According to the increased CD16⁺ population by BiNK, when assessing cell killing in the time different treatment schedules (FIG. 38A), BiNK (anti-HER2×anti-NKG2A) enhanced the ADCC activity of anti-HER2 IgG1 Pertuzumab (FIG. 38B).

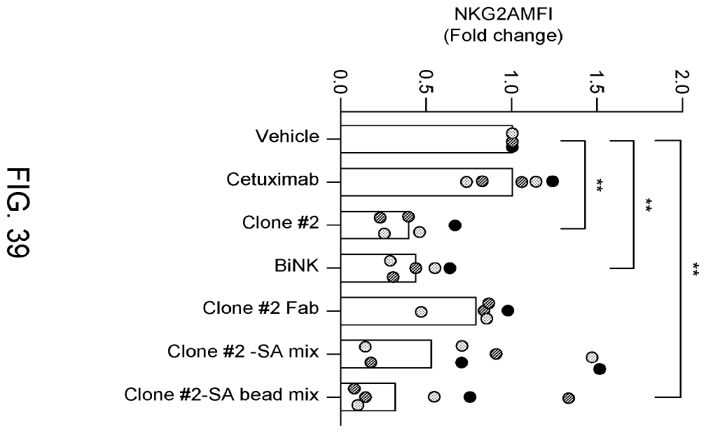

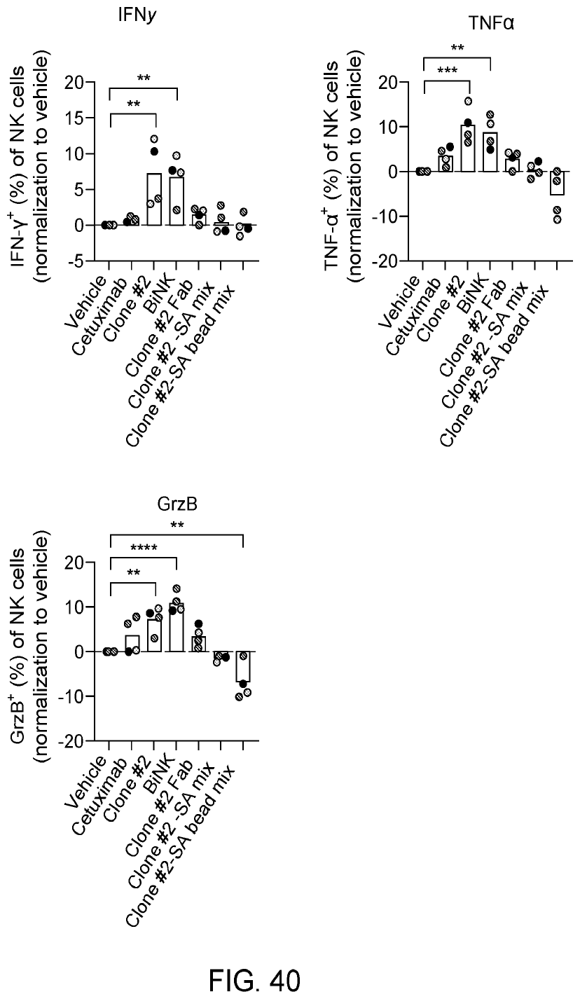

To evaluate how much NKG2A binding capacity is required for NK cell activation, the NKG2A binders were constructed with different NKG2A binding capacity. Both Clone #2 IgG (anti-NKG2A) and BiNK (anti-EGFR×anti-NKG2A), which has bivalent binding to NKG2A, downregulated NKG2A expression (FIG. 39) and increased the production of IFNγ, TNFα, and Granzyme B (GrzB) of primary NK cells (FIG. 40). Clone #2-SA bead mix with multivalent binding, which mimics HLA-E/NKG2A interaction, decreased TNFα, and GrzB (FIG. 40), even though it also decreased NKG2A expression level (FIG. 39). These results demonstrate that the bivalent binding to NKG2A is involved in NK cell activation.

### Example 5—Methods and Materials

### Purification of Fab and IgG1-LALA-PG (L234A, L235A, and P329G Mutations)

The plasmid was transformed into HB2151 E. coli competent cells, and then colonies were selected in ampicillin containing LB plate (100 μg/mL final concentration) for overnight in incubator at 37° C. Next day, a colony was inoculated in liquid LB+ampicillin media and cultured in 37° C. shaking incubator. 0.1 mM of isopropyl β-D-1-thiogalactopyranoside (IPTG) as final concentration was added to culture at OD₆₀₀ of between about 0.4-0.6 corresponding to around 4×10⁸ cells/mL. The culture was relocated to a shaking incubator set as 30° C., 200 rpm and incubated overnight. Next day, E. coli cells were harvested and resuspended in 1/10 volume of periplasm extraction buffer containing polymyxin B (0.5 mg/mL in PBS pH 7.4) and then incubated on ice for an hour. Supernatant was collected by centrifugation at 12,000×g for 10 minutes, then loaded into pre-packed Ni-NTA resin. The bound Fab was eluted by adding 300 mM imidazole in PBS pH 7.4, and then imidazole was removed by using PD-10 desalting column (GE, 45-000-148).

For IgG preparation, IgG cloned plasmid DNA was transfected to HEK239F cells and expressed for 5-7 days post-transfection. Expressed IgG was purified by affinity chromatography with protein A resin (Captiva, NC0997253). Elution of bound IgG was performed by adding 50 mM glycine buffer pH 3.0, and then storage buffer was changed to phospho-buffered saline pH 7.4 (PBS) by PD-10 desalting column. Protein purity was estimated in either SDS-PAGE or size exclusion chromatography packed with Superdex 200 increase 10/300 GL (GE healthcare, 28990944). The concentration of each protein was determined by Nano Drop spectrophotometer 2000C (Thermo, ND2000C).

### Binding of Fab or IgG in Enzyme-Linked Immunosorbent Assay (ELISA)

Binding and specificity of Fab or IgG to single chain CD94/NKG2A-Fc or single chain CD94/NKG2C-Fc were analyzed through indirect ELISA. Briefly, Fc-fused proteins were coated on a 96 well plate (Corning, 3690) at 200 ng/well (50 μL volume) in PBS for 2 hours at room temperature. Blocking was carried out with 3% BSA in PBS for overnight at 4° C. Next day, various concentration of Fab or IgG was applied to antigen coated plates and incubated for 1 hour at 25° C. After three times washing, anti-FLAG mouse antibody (M2 clone)-HRP conjugated (Sigma, A8592, 1:3000 dilution) or anti-human kappa goat antibody-HRP conjugated (Invitrogen, A18853, 1:3000 dilution) was applied to detect binding of either Fab or IgG. Same volume of TMB (Thermo, PI34028) was added as a substrate, and then enzymatic reactions were stopped by adding of 2N sulfonic acid.

### Cell Lines

H2030, A549, Farage, and 293T cells were purchased from ATCC. H2030, A549, and Farage cells were cultured RPMI 1640 (ATCC) supplemented with 10% v/v FBS (Gibco) and 1% penicillin-streptomycin (P/S, Gibco). 293T cells were maintained in DMEM (Gibco) supplemented with 10% v/v FBS and 1% P/S. Primary NK cells were cultured with MACS basal NK media with supplementary (Miltenyi Biotec, 130-114-429), 10% v/v human serum (Sigma-Aldrich), and hIL-2 (50 IU/mL, Miltenyi Biotec).

### Flow Cytometry Analysis

Primary NK cells were enriched from normal human peripheral blood mononuclear cells (PBMCs, Zenbio Inc.) using NK cell isolation kit (Miltenyi Biotec, 130-092-657). The purify of isolated NK cells was confirmed by staining of APC conjugated anti-human CD56 antibody and PE conjugated anti-human CD16 antibody. To determine the NKG2A⁺ population of primary NK cells, cells were stained with Alexa488 (AR488)-conjugated anti-NKG2A IgG1. To confirm the cell surface binding of antibodies (e.g., monalizumab, Clone #1 IgG, and Clone #2 IgG), cells were treated with antibodies (10 nM) for 1 hour at 4° C. and then stained with Alexa647-conjugated goat anti-human IgG (Invitrogen, A21445) for 0.5 hours at 4° C. For investigation of NKG2A expression level of NK cells, enriched NK cells were activated with 50 IU/mL of hIL-2 and treated with the indicated antibodies (100 nM) for 24 hours at 37° C. After incubation with antibodies, cells were washed with cold PBS, and then the cell surface NKG2A was detected using a FITC- or PE-conjugated anti-human CD159a (NKG2A) antibody. For gating of NK cells in the co-culture system with H2030 or A549 cells, APC conjugated anti-human CD45 antibody was used. Monoclonal antibodies specific for CD56 (12-0567-42), CD16 (12-0168-42), CD159a (130-113-566), and CD45 (17-0459-42) were purchased from Thermofisher. For intracellular staining, GolgiPlug containing brefeldin A (Invitrogen, 00-4506-51) was added for the final 4 hours of culture to inhibit protein transport from the endoplasmic reticulum to the Golgi apparatus.

All intracellular staining was performed using a BD cytofix/cytoperm kit (BD Biosciences, BD554714). Monoclonal antibodies specific for IFNγ (12-7319-42) and granzyme B (12-8896-42) were purchased from Thermofisher. Anti-PD-L1 antibody, durvalumab, was purchased from Selleckchem (A2013). Data were acquired using the flow cytometry BD LSR II (San Jose, CA) and analyzed with FlowJo 10.7.1 (Tree Star).

Functional Assay with Peripheral Human NK Cells

The purified human NK cells from PBMCs were used as effector cells. NK cells were incubated with target cell line (H2030 or A549) (E:T ratio, 5:1 or 2:1), in the presence of 50 IU/mL hIL-2 for 24 hours at 37° C. Cells were dispensed into 96-well plates with or without the indicated binders (e.g., monalizumab, Clone #1 IgG, Clone #2 IgG, durvalumab, or a BiNK) at 100 nM for 24 hours at 37° C. After treatment with the binders, cells were fixed and intracellular stained with PE-conjugated anti-human IFN7 antibody, PE-conjugated anti-human TNFα antibody and PE-conjugated anti-human granzyme B antibody. Cells were washed twice and analyzed on the BD LSR II flow cytometry.

### In Vitro Cytotoxicity Assay

The ADCC or cell killing activity of effector cells incubated with anti-NKG2A binders with the LALA-PG mutation were measured through release of cytosolic LDH from dead target cells using LDH-Glo cytotoxicity assay kit (Promega, J2381). The purified NK cells from normal PBMCs (Zenbio Inc.) were used as effector cells and were incubated with A549 or 293T cells (1×10⁴ cells/well in 96-well plate) as target cells at effector-to-target (E:T) ratios of 5:1 or 2:1, in the presence of the indicated binders for 4 hours at 37° C. Percent cytotoxicity was calculated with the following formula: 100×(experimental-effector minimum-target minimum)/(target maximum-target minimum).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions).

2-10. (canceled)

11. An antigen binding fragment comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions).

12-24. (canceled)

25. A chimeric antigen receptor comprising an antigen binding domain, a hinge, a transmembrane domain, and one or more signaling domains, wherein said antigen binding domain comprises:
(i) a heavy chain variable domain or region comprising the amino acid sequences set forth in SEO ID NO:1 (or SEO ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions).

26-31. (canceled)

32. A cell engager comprising a first antigen binding domain, a linker, and a second antigen binding domain, wherein said first antigen binding domain comprises:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEO ID NO:9 (or SEO ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions).

33-45. (canceled)

46. A nucleic acid comprising a nucleic acid sequence encoding at least part of an antibody or an antigen-binding fragment comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or
(ii) a heavy chain variable domain or region comprising the amino acid sequences set forth in SEO ID NO:17 (or SEO ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions).

47-50. (canceled)

51. A nucleic acid comprising a nucleic acid sequence encoding a cell engager of claim 32.

52-54. (canceled)

55. A host cell that expresses a chimeric antigen receptor of claim 25.

56. (canceled)

57. An antibody-drug conjugate (ADC) comprising an antigen binding domain covalently linked to a drug, wherein said antigen binding domain comprises:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEO ID NO:3 (or SEO ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions).

58-60. (canceled)

61. A composition comprising an antibody or an antigen binding fragment comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEO ID NO:11 with one, two, or three amino acid additions, deletions, or substitutions); or
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions).

62-63. (canceled)

64. A composition comprising a cell engager of claim 32.

65-68. (canceled)

69. A method of treating a mammal having cancer or a viral infection, wherein said method comprises administering, to said mammal, a composition comprising a cell engager of claim 32, a cell of claim 55, an ADC of claim 57, or an antibody or an antigen binding fragment comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEO ID NO:10 (or SEO ID NO:10 with one, two, or three amino acid additions, deletions, or substitutions), and SEQ ID NO:11 (or SEQ ID NO:11 with one, two, or three amino acid additions, deletions, or substitutions); or
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions).

70-84. (canceled)

85. A method for binding a binding molecule to a CD94/NKG2A polypeptide, wherein said method comprises contacting said CD94/NKG2A polypeptide with a chimeric antigen receptor of, a cell engager of claim 32, an ADC of claim 57, or an antibody or an antigen binding fragment comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEO ID NO:11 with one, two, or three amino acid additions, deletions, or substitutions); or
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions).

86-89. (canceled)

90. A composition comprising an antibody or antibody binding fragment, wherein said antibody or said antibody binding fragment comprises the ability to bind to a CD94/NKG2A polypeptide present on an NK cell or T cell without inhibiting the cytotoxic activity of said NK cell or said T cell.

91-94. (canceled)

95. A composition comprising an antibody or antibody binding fragment, wherein said antibody or said antibody binding fragment comprises the ability to bind to a CD94/NKG2A polypeptide present on an NK cell or T cell and to reduce expression of CD94/NKG2A polypeptides on the surface of said NK cell or said T cell.

96-99. (canceled)

100. A cell engager comprising a first antigen binding domain, a linker, and a second antigen binding domain, wherein said first antigen binding domain comprises the ability to bind to a CD94/NKG2A polypeptide present on an NK cell or T cell without inhibiting the cytotoxic activity of said NK cell or said T cell.

101-114. (canceled)

115. A method for binding a molecule to a CD94/NKG2A polypeptide on the surface of an NK cell or a T cell without inhibiting the cytotoxic activity of said NK cell or said T cell, wherein said method comprises contacting said NK cell or said T cell with a cell engager of claim 100.

116-119. (canceled)

120. A method for reducing expression of CD94/NKG2A polypeptides on the surface of a NK cell or a T cell, wherein said method comprises contacting said NK cell or said T cell with a cell engager of claim 100.

121-124. (canceled)

125. A method for killing HER2⁺ cancer cells, wherein said method comprises contacting immune cells positive for CD94/NKG2A polypeptides with (a) a cell engager of claim 100 and (b) an anti-HER2 antibody.

126-130. (canceled)

131. A method treating a mammal having cancer, wherein said method comprises administering a cell engager of claim 100 to said mammal.

132-135. (canceled)


SEQUENCE LISTING (326 sequences)

SEQ ID NO: 1 - protein, 5 aa, synthetic construct
DYYMS

SEQ ID NO: 2 - protein, 17 aa, synthetic construct
YISSSGSTIYYADSVKG

SEQ ID NO: 3 - protein, 6 aa, synthetic construct
GFYMDA

SEQ ID NO: 4 - protein, 30 aa, synthetic construct
QVQLVESGGGLVKPGGSLRLSCAASGFTFS

SEQ ID NO: 5 - protein, 14 aa, synthetic construct
WIRQAPGKGLEWVS

SEQ ID NO: 6 - protein, 32 aa, synthetic construct
RFTISRDNAKNSLYLQMNSLRAEDTAVYYCAR

SEQ ID NO: 7 - protein, 11 aa, synthetic construct
WGKGTTVTVSS

SEQ ID NO: 8 - protein, 115 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGFYMDAWGKGTTVTVSS

SEQ ID NO: 9 - protein, 11 aa, synthetic construct
RASQSISSYLN

SEQ ID NO: 10 - protein, 7 aa, synthetic construct
AASSLQS

SEQ ID NO: 11 - protein, 10 aa, synthetic construct
QYYGISPPFT

SEQ ID NO: 12 - protein, 23 aa, synthetic construct
DIQMTQSPSSLSASVGDRVTITC

SEQ ID NO: 13 - protein, 15 aa, synthetic construct
WYQQKPGKAPKLLIY

SEQ ID NO: 14 - protein, 32 aa, synthetic construct
GVPSRFSGSGSGTDFTLTISSLQPEDFGVYYC

SEQ ID NO: 15 - protein, 11 aa, synthetic construct
FGPGTKVDIKR

SEQ ID NO: 16 - protein, 109 aa, synthetic construct
DIQMTQSPSSLSASVGDRVTITCRASQSISSYLNWYQQKPGKAPKLLIYAASSLQSGVPSRFSGSGSGTDFTLTISSLQPEDFGVYYCQYYGISPPFTFGPGTKVDIKR

SEQ ID NO: 17 - protein, 5 aa, synthetic construct
GYYMS

SEQ ID NO: 18 - protein, 17 aa, synthetic construct
YISNSGRTIYYADSVKG

SEQ ID NO: 19 - protein, 6 aa, synthetic construct
GFYMDA

SEQ ID NO: 20 - protein, 30 aa, synthetic construct
EVQLVESGGGLVKPGGSLRLSCAASGFTFS

SEQ ID NO: 21 - protein, 14 aa, synthetic construct
WIRQAPGKGLEWLA

SEQ ID NO: 22 - protein, 32 aa, synthetic construct
RFTISRDNSNSSLYLQMNSLRAEDTAVYYCAR

SEQ ID NO: 23 - protein, 11 aa, synthetic construct
WGKGTTVTVSS

SEQ ID NO: 24 - protein, 115 aa, synthetic construct
EVQLVESGGGLVKPGGSLRLSCAASGFTFSGYYMSWIRQAPGKGLEWLAYISNSGRTIYYADSVKGRFTISRDNSNSSLYLQMNSLRAEDTAVYYCARGFYMDAWGKGTTVTVSS

SEQ ID NO: 25 - protein, 11 aa, synthetic construct
RASQSISSYLN

SEQ ID NO: 26 - protein, 7 aa, synthetic construct
AASSLQS

SEQ ID NO: 27 - protein, 10 aa, synthetic construct
QYYGISPPFT

SEQ ID NO: 28 - protein, 23 aa, synthetic construct
DIQMTQSPSSLSASVGDRVTITC

SEQ ID NO: 29 - protein, 15 aa, synthetic construct
WYQQKPGKAPKLLIY

SEQ ID NO: 30 - protein, 32 aa, synthetic construct; identical to SEQ ID NO: 14

SEQ ID NO: 31 - protein, 11 aa, synthetic construct
FGPGTKVDIKR

SEQ ID NO: 32 - protein, 109 aa, synthetic construct; identical to SEQ ID NO: 16

SEQ ID NO: 33 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 34 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - DNA, 345 bp, synthetic construct
  source 1..345: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggctgtgtattactgtgcaagggggttctacatggacgcctggggcaaagggaccacggtcaccgtctcctca

SEQ ID NO: 36 - DNA, 327 bp, synthetic construct
  source 1..327: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaggattttggagtttattactgtcagtactatggtatttcacccccattcactttcggccctgggaccaaggtggatatcaaacgt

SEQ ID NO: 37 - DNA, 345 bp, synthetic construct
  source 1..345: mol_type = other DNA
gaggtgcagctggtggagtctgggggaggcttggtcaagcctggagggtccctgagactgtcatgtgcagcctctggattcacattcagtgggtactacatgagttggatccgccaggctccagggaaggggctggagtggcttgcatacatcagtaatagtggaagaaccatctactacgcagactctgtgaagggccgattcaccatctccagggacaactccaacagctcactgtatttgcaaatgaacagcctgagagccgaggacacggcagtgtactactgcgcgagaggcttctatatggatgcgtggggaaaagggactacggtaactgttagttct

SEQ ID NO: 38 - DNA, 327 bp, synthetic construct; identical to SEQ ID NO: 36
  source 1..327: mol_type = other DNA

SEQ ID NO: 39 - protein, 15 aa, Homo sapiens
EPKSCDKTHTCPPCP

SEQ ID NO: 40 - DNA, 45 bp, Homo sapiens
  source 1..45: mol_type = other DNA
gagcccaaatcttgtgacaaaactcacacatgcccaccgtgccca

SEQ ID NO: 41 - DNA, 651 bp, Homo sapiens
  source 1..651: mol_type = other DNA
gcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtataccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 42 - protein, 217 aa, Homo sapiens
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43-44 - skipped (2 entries)

SEQ ID NO: 45 - protein, 249 aa, synthetic construct
DIQMTQSPSSLSASVGDRVTITCRASQSISSYLNWYQQKPGKAPKLLIYAASSLQSGVPSRFSGSGSGTDFTLTISSLQPEDFGVYYCQYYGISPPFTFGPGTKVDIKRGGGGSGGGGSGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGFYMDAWGKGTTVTVSS

SEQ ID NO: 46 - protein, 239 aa, synthetic construct
DIQMTQSPSSLSASVGDRVTITCRASQSISSYLNWYQQKPGKAPKLLIYAASSLQSGVPSRFSGSGSGTDFTLTISSLQPEDFGVYYCQYYGISPPFTFGPGTKVDIKRGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGFYMDAWGKGTTVTVSS

SEQ ID NO: 47 - protein, 249 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGFYMDAWGKGTTVTVSSGGGGSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFGVYYCQYYGISPPFTFGPGTKVDIKR

SEQ ID NO: 48 - protein, 239 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGFYMDAWGKGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFGVYYCQYYGISPPFTFGPGTKVDIKR

SEQ ID NO: 49-50 - skipped (2 entries)

SEQ ID NO: 51 - protein, 239 aa, synthetic construct
DIQMTQSPSSLSASVGDRVTITCRASQSISSYLNWYQQKPGKAPKLLIYAASSLQSGVPSRFSGSGSGTDFTLTISSLQPEDFGVYYCQYYGISPPFTFGPGTKVDIKRGGGGSGGGGSGGGGSEVQLVESGGGLVKPGGSLRLSCAASGFTFSGYYMSWIRQAPGKGLEWLAYISNSGRTIYYADSVKGRFTISRDNSNSSLYLQMNSLRAEDTAVYYCARGFYMDAWGKGTTVTVSS

SEQ ID NO: 52 - protein, 239 aa, synthetic construct
EVQLVESGGGLVKPGGSLRLSCAASGFTFSGYYMSWIRQAPGKGLEWLAYISNSGRTIYYADSVKGRFTISRDNSNSSLYLQMNSLRAEDTAVYYCARGFYMDAWGKGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFGVYYCQYYGISPPFTFGPGTKVDIKR

SEQ ID NO: 53 - skipped

SEQ ID NO: 54 - protein, 25 aa, synthetic construct
EVQLVQSGGGLVQPGGSLRLSCSAS

SEQ ID NO: 55 - protein, 17 aa, synthetic construct
MHWVRQAPGKGLEYVSA

SEQ ID NO: 56 - protein, 38 aa, synthetic construct
YYADSVKGRFTISRDNSKNTLYLQMNSLRAEDTAVYYC

SEQ ID NO: 57 - protein, 11 aa, synthetic construct
WGQGTMVTVSS

SEQ ID NO: 58 - skipped

SEQ ID NO: 59 - protein, 26 aa, synthetic construct
EATLTQSPSSLSASVGDRVTITCRAS

SEQ ID NO: 60 - protein, 17 aa, synthetic construct
LNWYQQKPGRAPKLLIY

SEQ ID NO: 61 - protein, 36 aa, synthetic construct
SLQSGVPSRFSGSGSGTDFTLTISSLQPEDFATYYC

SEQ ID NO: 62 - protein, 11 aa, synthetic construct
FGQGTRLEIKR

SEQ ID NO: 63 - skipped

SEQ ID NO: 64 - protein, 25 aa, synthetic construct
EVQLVQSGGGLVQPGGSLRLSCAAS

SEQ ID NO: 65 - protein, 17 aa, synthetic construct
MTWVRQAPGKGLEWVST

SEQ ID NO: 66 - protein, 38 aa, synthetic construct
YYADSVKGRFTISRDNDKNTLYLQMNSLRADDTAVYYC

SEQ ID NO: 67 - protein, 11 aa, synthetic construct
WGQGTLVTVSS

SEQ ID NO: 68 - skipped

SEQ ID NO: 69 - protein, 25 aa, synthetic construct
QSVVTQPPSASGSPGQSVTISCTGT

SEQ ID NO: 70 - protein, 17 aa, synthetic construct
VSWYQQHPGKAPKLLIY

SEQ ID NO: 71 - protein, 36 aa, synthetic construct
ERPSGVPDRFSGSKSDNKASLTIIGLQTEDEADYYC

SEQ ID NO: 72 - protein, 11 aa, synthetic construct
FGTGTKLTVLR

SEQ ID NO: 73 - protein, 8 aa, Homo sapiens
EPKSCDKT

SEQ ID NO: 74 - protein, 179 aa, Homo sapiens
MAVFKTTLWRLISGTLGIICLSLMSTLGILLKNSFTKLSIEPAFTPGPNIELQKDSDCCSCQEKWVGYRCNCYFISSEQKTWNESRHLCASQKSSLLQLQNTDELDFMSSSQQFYWIGLSYSEEHTAWLWENGSALSQYLFPSFETFNTKNCIAYNPNGNALDESCEDKNRYICKQQLI

SEQ ID NO: 75 - protein, 233 aa, Homo sapiens
MDNQGVIYSDLNLPPNPKRQQRKPKGNKNSILATEQEITYAELNLQKASQDFQGNDKTYHCKDLPSAPEKLIVGILGIICLILMASVVTIVVIPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 76 - protein, 15 aa, synthetic construct
GGGGSGGSGSGGGGS

SEQ ID NO: 77 - protein, 15 aa, synthetic construct
GGGGSGGSGSSGGGS

SEQ ID NO: 78 - protein, 15 aa, synthetic construct
GGGGSGGGGSGGGGS

SEQ ID NO: 79 - protein, 15 aa, synthetic construct
GGGGSGSGASSGGGS

SEQ ID NO: 80 - protein, 15 aa, synthetic construct
GGGGSGSGASGGGGS

SEQ ID NO: 81 - protein, 15 aa, synthetic construct
SGGGSGGGGSGGGGS

SEQ ID NO: 82 - protein, 18 aa, synthetic construct
GSTSGSGKPGSGEGSTKG

SEQ ID NO: 83 - protein, 19 aa, synthetic construct
PNGASQSSSASHTGSAPGS

SEQ ID NO: 84 - protein, 19 aa, synthetic construct
PNGASNSGSAPDTSSAPGS

SEQ ID NO: 85 - protein, 19 aa, synthetic construct
PNGASHSGSAPNTSSAPGS

SEQ ID NO: 86 - protein, 19 aa, synthetic construct
PNGASESGSASKTSSASGS

SEQ ID NO: 87 - protein, 19 aa, synthetic construct
PNGASNSGSAPKTGSASGS

SEQ ID NO: 88 - protein, 19 aa, synthetic construct
PNGASKSGSASQTSSAPGS

SEQ ID NO: 89 - protein, 19 aa, synthetic construct
PNGASHSSSASQTGSAPGS

SEQ ID NO: 90 - protein, 12 aa, synthetic construct
PNGASKRSAPGS

SEQ ID NO: 91 - protein, 19 aa, synthetic construct
PNGASHSGSAPHTSSASGS

SEQ ID NO: 92 - protein, 15 aa, synthetic construct
RGRGRGRGRSRGGGS

SEQ ID NO: 93 - protein, 15 aa, synthetic construct
SHGGSHGGGSGGGGS

SEQ ID NO: 94 - protein, 5 aa, synthetic construct
GQAGR

SEQ ID NO: 95 - protein, 8 aa, synthetic construct
GGGSGGGG

SEQ ID NO: 96 - protein, 5 aa, synthetic construct
SGGGG

SEQ ID NO: 97 - protein, 5 aa, synthetic construct
GGGGS

SEQ ID NO: 98 - protein, 30 aa, synthetic construct
GGGGSGGGGSGGGGSGGGGSGGGGSGGGGS

SEQ ID NO: 99 - protein, 16 aa, synthetic construct
GGGGGSGGGGSGGGGS

SEQ ID NO: 100 - protein, 26 aa, synthetic construct
GGGGGSGGGGSGGGGSGGGGSGGGGS

SEQ ID NO: 101 - protein, 25 aa, synthetic construct
GGGGSGGGGSGGGGSGGGGSGGGGS

SEQ ID NO: 102 - skipped

SEQ ID NO: 103 - protein, 22 aa, synthetic construct
MDMRVPAQLLGLLLLWLRGARC

SEQ ID NO: 104 - DNA, 66 bp, synthetic construct
  source 1..66: mol_type = other DNA
atggatatgagggtccccgcacaattgctcggtctccttctgctttggctccggggcgcgcggtgc

SEQ ID NO: 105 - protein, 22 aa, synthetic construct
MDMRVLAQLLGLLLLCFPGARC

SEQ ID NO: 106 - protein, 20 aa, synthetic construct
MEAPAQLLFLLLLWLPDTTG

SEQ ID NO: 107 - protein, 20 aa, synthetic construct
MVLQTQVFISLLLWISGAYG

SEQ ID NO: 108 - protein, 19 aa, synthetic construct
MLPSQLIGFLLLWVPASRG

SEQ ID NO: 109 - protein, 22 aa, synthetic construct
MDMRVPAQLLGLLLLWLSGARC

SEQ ID NO: 110 - protein, 15 aa, synthetic construct
EPKSCDKTHTCPPCP

SEQ ID NO: 111 - protein, 15 aa, synthetic construct
DKTHTCPPCPAPPVA

SEQ ID NO: 112 - protein, 12 aa, synthetic construct
ESKYGPPCPPCP

SEQ ID NO: 113 - protein, 12 aa, synthetic construct
ESKYGPPCPSCP

SEQ ID NO: 114 - protein, 49 aa, synthetic construct
KPTTTPAPRPPTPAPTIASQPLSLRPEACRPAAGGAVHTRGLDFACDIY

SEQ ID NO: 115 - protein, 39 aa, synthetic construct
IEVMYPPPYLDNERSNGTIIHVKGKHLCPSPLFPGPSKP

SEQ ID NO: 116 - protein, 47 aa, synthetic construct
TTTPAPRPPTPAPTIASQPLSLRPEACRPAAGGAVHTRGLDFACDIY

SEQ ID NO: 117 - protein, 45 aa, synthetic construct
TTTPAPRPPTPAPTIASQPLSLRPEACRPAAGGAVHTRGLDFACD

SEQ ID NO: 118 - protein, 21 aa, synthetic construct
LCYLLDGILFIYGVILTALFL

SEQ ID NO: 119 - protein, 22 aa, synthetic construct
MALIVLGGVAGLLLFIGLGIFF

SEQ ID NO: 120 - protein, 21 aa, synthetic construct
IYIWAPLAGTCGVLLLSLVIT

SEQ ID NO: 121 - protein, 24 aa, synthetic construct
IYIWAPLAGTCGVLLLSLVITLYC

SEQ ID NO: 122 - protein, 22 aa, synthetic construct
IWAPLAGTCGVLLLSLVITLYC

SEQ ID NO: 123 - protein, 19 aa, synthetic construct
IWAPLAGTCGVLLLSLVIT

SEQ ID NO: 124 - protein, 27 aa, synthetic construct
FWVLVVVGGVLACYSLLVTVAFIIFWV

SEQ ID NO: 125 - protein, 21 aa, synthetic construct
FWLPIGCAAFVVVCILGCILI

SEQ ID NO: 126 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 127 - protein, 42 aa, synthetic construct
KRGRKKLLYIFKQPFMRPVQTTQEEDGCSCRFPEEEEGGCEL

SEQ ID NO: 128 - protein, 37 aa, synthetic construct
KRGRKKLLYIFKQPFMRPVQTTQEEDGCSCRFPEEEE

SEQ ID NO: 129 - protein, 41 aa, synthetic construct
RSKRSRLLHSDYMNMTPRRPGPTRKHYQPYAPPRDFAAYRS

SEQ ID NO: 130 - protein, 42 aa, synthetic construct
ALYLLRRDQRLPPDAHKPPGGGSFRTPIQEEQADAHSTLAKI

SEQ ID NO: 131 - protein, 38 aa, synthetic construct
CWLTKKKYSSSVHDPNGEYMFMRAVNTAKKSRLTDVTL

SEQ ID NO: 132 - protein, 24 aa, synthetic construct
LCARPRRSPAQEDGKVYINMPGRG

SEQ ID NO: 133 - protein, 52 aa, synthetic construct
YFLGRLVPRGRGAAEAATRKQRITETESPYQELQGQRSDVYSDLNTQRPYYK

SEQ ID NO: 134 - protein, 48 aa, synthetic construct
QRRKYRSNKGESPVEPAEPCHYSCPREEEGSTIPIQEDYRKPEPACSP

SEQ ID NO: 135 - protein, 509 aa, synthetic construct
MDMRVPAQLLGLLLLWLRGARCDIQMTQSPSSLSASVGDRVTITCRASQSISSYLNWYQQKPGKAPKLLIYAASSLQSGVPSRFSGSGSGTDFTLTISSLQPEDFGVYYCQYYGISPPFTFGPGTKVDIKRGGGGSGGGGSGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGFYMDAWGKGTTVTVSSAAAESKYGPPCPSCPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGGGSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 136 - protein, 499 aa, synthetic construct
MDMRVPAQLLGLLLLWLRGARCDIQMTQSPSSLSASVGDRVTITCRASQSISSYLNWYQQKPGKAPKLLIYAASSLQSGVPSRFSGSGSGTDFTLTISSLQPEDFGVYYCQYYGISPPFTFGPGTKVDIKRGGGGSGGGGSGGGGSEVQLVESGGGLVKPGGSLRLSCAASGFTFSGYYMSWIRQAPGKGLEWLAYISNSGRTIYYADSVKGRFTISRDNSNSSLYLQMNSLRAEDTAVYYCARGFYMDAWGKGTTVTVSSAAAESKYGPPCPSCPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGGGSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 137 - protein, 256 aa, synthetic construct
DIQMTQSPSSLSASVGDRVTITCRASQSISSYLNWYQQKPGKAPKLLIYAASSLQSGVPSRFSGSGSGTDFTLTISSLQPEDFGVYYCMQALQTPVTFGQGTRLEIKRGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARIGRYSYGLRSYYYYGMDVWGQGTTVTVSSGSGGGS

SEQ ID NO: 138 - DNA, 768 bp, synthetic construct
  source 1..768: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaggattttggggtttattactgcatgcaagctctacaaactccggtcaccttcggccaagggacacgactggagattaaacgtggcggtggagggagcggtggcgggggaagtggagggggtggcagccaagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggctgtgtattactgtgcacggattgggcggtacagctatggtctccggtcctactactactacggtatggacgtctggggccaagggaccacggtcaccgtctcctcaggaagcggcggaggatca

SEQ ID NO: 139 - protein, 119 aa, synthetic construct [sequence omitted]

SEQ ID NO: 140 - protein, 107 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 141 - protein, 122 aa, synthetic construct
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 142 - protein, 108 aa, synthetic construct
MDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 143 - protein, 117 aa, synthetic construct
EVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSR

SEQ ID NO: 144 - protein, 108 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVG

SEQ ID NO: 145 - protein, 125 aa, synthetic construct
QVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSS

SEQ ID NO: 146 - protein, 107 aa, synthetic construct
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIK

SEQ ID NO: 147 - protein, 119 aa, synthetic construct
EVQLVESGGGLVKPGGSLRLSCAASGFTFSSYAMSWVRQAPGKGLEWVSEISSGGSYTYYADSVKGRFTISRDNAKNSLYLQMNSLRAEDTAVYYCARHGDYPRFFDVWGQGTTVTVSS

SEQ ID NO: 148 - protein, 107 aa, synthetic construct
EIVLTQSPATLSLSPGERATLSCSASSSVSSYIYWYQQKPGQAPRLLIYLTSNLASGIPARFSGSGSGTDFTLTISSLEPEDFAVYYCQQWSGNPYTFGQGTKLEIK

SEQ ID NO: 149 - protein, 121 aa, synthetic construct
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 150 - protein, 110 aa, synthetic construct
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 151 - protein, 114 aa, synthetic construct
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTLVTVSS

SEQ ID NO: 152 - protein, 114 aa, synthetic construct
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTPVTVSS

SEQ ID NO: 153 - protein, 107 aa, synthetic construct [sequence omitted]

SEQ ID NO: 154 - protein, 114 aa, synthetic construct
EIQLQQSGAELVKPGASVKLSCTASGFNIKDTYFHWVKQRPEQGLEWIGRIDPANGNTKYDPKFHDKATIIADISSNTAYLQFSSLTSEDTAVYYCAANRYGYWGQGTTLTVSS

SEQ ID NO: 155 - protein, 112 aa, synthetic construct
DIVMTQAAPSIPVTPGESVSISCRSSKSLLYINGNTHLFWFLQRPGQSPQLLIYRMSNLASGVPDRFSGSGSGTAFTLRISRVEAEDVGVYYCMQHLEYPFTFGSGTKLEIK

SEQ ID NO: 156 - protein, 120 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 157 - protein, 107 aa, synthetic construct
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 158 - protein, 113 aa, synthetic construct
EVQLVESGGGLVQPGGSLRLSCAASGFTFSNYGMSWVRQAPGKGLEWIGSIYYSGSTNYNPSLKSLVTISRDNSKNTLYLQMNSLRAEDTATYYCARESIDYWGQGTLVTVSS

SEQ ID NO: 159 - protein, 486 aa, synthetic construct
DIQMTQSPSSLSASVGDRVTITCRASQSISSYLNWYQQKPGKAPKLLIYAASSLQSGVPSRFSGSGSGTDFTLTISSLQPEDFGVYYCQYYGISPPFTFGPGTKVDIKRGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGFYMDAWGKGTTVTVSSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 160 - DNA, 1335 bp, synthetic construct
  source 1..1335: mol_type = other DNA
caagtccaacttgtggaatcaggcggtggtcttgttaagccgggtggaagtctccggctttcctgtgccgcctccggttttacttttagcgactactatatgagttggattcgccaagcccctggcaaagggcttgaatgggtttcctatatttctagtagcggcagtaccatatattacgccgactccgtaaaggggcgattcacgattagtagagataacgctaagaactctctgtatttgcaaatgaactctctgcgggcagaggacaccgcagtgtactactgcgcgagaggcttctatatggatgcgtggggaaaagggactacggtaactgttagttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtataccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 161 - protein, 445 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGFYMDAWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - DNA, 1335 bp, synthetic construct
  source 1..1335: mol_type = other DNA
gaggtgcagctggtggagtctgggggaggcttggtcaagcctggagggtccctgagactgtcatgtgcagcctctggattcacattcagtgggtactacatgagttggatccgccaggctccagggaaggggctggagtggcttgcatacatcagtaatagtggaagaaccatctactacgcagactctgtgaagggccgattcaccatctccagggacaactccaacagctcactgtatttgcaaatgaacagcctgagagccgaggacacggcagtgtactactgcgcgagaggcttctatatggatgcgtggggaaaagggactacggtaactgttagttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtataccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 163 - protein, 445 aa, synthetic construct
EVQLVESGGGLVKPGGSLRLSCAASGFTFSGYYMSWIRQAPGKGLEWLAYISNSGRTIYYADSVKGRFTISRDNSNSSLYLQMNSLRAEDTAVYYCARGFYMDAWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - DNA, 645 bp, synthetic construct
  source 1..645: mol_type = other DNA
gacatacagatgactcaatctccaagctcattgtccgcgtctgtcggtgacagagtcacgattacatgccgggcttctcagtccatttcatcatatctcaactggtatcaacagaaacccggtaaggctccaaagctccttatttatgccgcatcctctctgcagagcggggttccgagccggttcagtggaagtggctcaggaactgatttcacattgacaatatctagcctccagccggaagattttggggtgtactactgccagtattatggcatctctccaccgttcacgtttggcccaggaacgaaagtagacataaagcgtacggtcgctgcgcccagtgtatttatctttcctcccagcgatgagcaactcaaaagtggaaccgccagtgtggtgtgtttgttgaataacttttatcccagagaagctaaggtacaatggaaggtcgataacgcccttcagtctggaaatagtcaggaaagtgtcacagagcaggattccaaggactccacgtatagtcttagtagtaccttgactctgagcaaagcagactatgagaagcacaaggtgtatgcttgtgaagtgacgcaccagggcctctcctcccccgtgacgaagagctttaatcgcggagagtgc

SEQ ID NO: 165 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFGVYYCQYYGISPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 5 aa, synthetic construct
SYYMS

SEQ ID NO: 167 - protein, 5 aa, synthetic construct
DYAMS

SEQ ID NO: 168 - protein, 5 aa, synthetic construct
DYYTS

SEQ ID NO: 169 - protein, 5 aa, synthetic construct
DYYMH

SEQ ID NO: 170 - protein, 5 aa, synthetic construct
NYYMS

SEQ ID NO: 171 - protein, 5 aa, synthetic construct
DNYMS

SEQ ID NO: 172 - protein, 5 aa, synthetic construct
DYGMS

SEQ ID NO: 173 - protein, 5 aa, synthetic construct
DYYMN

SEQ ID NO: 174 - protein, 5 aa, synthetic construct
GYYMS

SEQ ID NO: 175 - protein, 5 aa, synthetic construct
DAYMS

SEQ ID NO: 176 - protein, 17 aa, synthetic construct
VISSSGSTIYYADSVKG

SEQ ID NO: 177 - protein, 16 aa, synthetic construct
YVSSGSTIYYADSVKG

SEQ ID NO: 178 - protein, 17 aa, synthetic construct
YIGSSGSTIYYADSVKG

SEQ ID NO: 179 - protein, 17 aa, synthetic construct
YISYSGSTIYYADSVKG

SEQ ID NO: 180 - protein, 17 aa, synthetic construct
YISSDGSTIYYADSVKG

SEQ ID NO: 181 - protein, 17 aa, synthetic construct
YISSSSSTIYYADSVKG

SEQ ID NO: 182 - protein, 17 aa, synthetic construct
YISSSGGTIYYADSVKG

SEQ ID NO: 183 - protein, 17 aa, synthetic construct
YISSSGSSIYYADSVKG

SEQ ID NO: 184 - protein, 17 aa, synthetic construct
YISSSGSTTYYADSVKG

SEQ ID NO: 185 - protein, 17 aa, synthetic construct
YISSSGSTIHYADSVKG

SEQ ID NO: 186 - protein, 6 aa, synthetic construct
GWYMDA

SEQ ID NO: 187 - protein, 6 aa, synthetic construct
GYYMDA

SEQ ID NO: 188 - protein, 6 aa, synthetic construct
GLYMDA

SEQ ID NO: 189 - protein, 6 aa, synthetic construct
GIYMDA

SEQ ID NO: 190 - protein, 6 aa, synthetic construct
SFYMDA

SEQ ID NO: 191 - protein, 6 aa, synthetic construct
AFYMDA

SEQ ID NO: 192 - protein, 6 aa, synthetic construct
VFYMDA

SEQ ID NO: 193 - protein, 6 aa, synthetic construct
GHYMDA

SEQ ID NO: 194 - protein, 6 aa, synthetic construct
GWHMDA

SEQ ID NO: 195 - protein, 6 aa, synthetic construct
GWMMDA

SEQ ID NO: 196 - protein, 11 aa, synthetic construct
QASQSISSYLN

SEQ ID NO: 197 - protein, 11 aa, synthetic construct
RMSQSISSYLN

SEQ ID NO: 198 - protein, 11 aa, synthetic construct
RARQSISSYLN

SEQ ID NO: 199 - protein, 11 aa, synthetic construct
RASESISSYLN

SEQ ID NO: 200 - protein, 11 aa, synthetic construct
RASQGISSYLN

SEQ ID NO: 201 - protein, 11 aa, synthetic construct
RASQSIRSYLN

SEQ ID NO: 202 - protein, 11 aa, synthetic construct
RASQSISNYLN

SEQ ID NO: 203 - protein, 11 aa, synthetic construct
RASQSISSWLN

SEQ ID NO: 204 - protein, 11 aa, synthetic construct
RASQSISSYLA

SEQ ID NO: 205 - protein, 11 aa, synthetic construct
WASQSISSYLN

SEQ ID NO: 206 - protein, 7 aa, synthetic construct
DASSLQS

SEQ ID NO: 207 - protein, 7 aa, synthetic construct
AAKSLQS

SEQ ID NO: 208 - protein, 7 aa, synthetic construct
AASTLQS

SEQ ID NO: 209 - protein, 7 aa, synthetic construct
AASSLES

SEQ ID NO: 210 - protein, 7 aa, synthetic construct
AASSLQT

SEQ ID NO: 211 - protein, 7 aa, synthetic construct
SASSLQS

SEQ ID NO: 212 - protein, 7 aa, synthetic construct
AAPSLQS

SEQ ID NO: 213 - protein, 7 aa, synthetic construct
AASNLQS

SEQ ID NO: 214 - protein, 7 aa, synthetic construct
AASSLHS

SEQ ID NO: 215 - protein, 7 aa, synthetic construct
AASSLQP

SEQ ID NO: 216 - protein, 10 aa, synthetic construct
QQYGISPPFT

SEQ ID NO: 217 - protein, 10 aa, synthetic construct
QHYGISPPFT

SEQ ID NO: 218 - protein, 10 aa, synthetic construct
QYHGISPPFT

SEQ ID NO: 219 - protein, 10 aa, synthetic construct
QSYGISPPFT

SEQ ID NO: 220 - protein, 10 aa, synthetic construct
QYSGISPPFT

SEQ ID NO: 221 - protein, 10 aa, synthetic construct
QYYAISPPFT

SEQ ID NO: 222 - protein, 10 aa, synthetic construct
QYYSISPPFT

SEQ ID NO: 223 - protein, 10 aa, synthetic construct
QYYGITPPFT

SEQ ID NO: 224 - protein, 10 aa, synthetic construct
QYYTISPPFT

SEQ ID NO: 225 - protein, 10 aa, synthetic construct
QYYGIYPPFT

SEQ ID NO: 226 - protein, 5 aa, synthetic construct
AYYMS

SEQ ID NO: 227 - protein, 5 aa, synthetic construct
SYYMS

SEQ ID NO: 228 - protein, 5 aa, synthetic construct
TYYMS

SEQ ID NO: 229 - protein, 5 aa, synthetic construct
GSYMS

SEQ ID NO: 230 - protein, 5 aa, synthetic construct
GTYMS

SEQ ID NO: 231 - protein, 5 aa, synthetic construct
GHYMS

SEQ ID NO: 232 - protein, 5 aa, synthetic construct
GYSMS

SEQ ID NO: 233 - protein, 5 aa, synthetic construct
GYTMS

SEQ ID NO: 234 - protein, 5 aa, synthetic construct
GYHMS

SEQ ID NO: 235 - protein, 5 aa, synthetic construct
GYYMT

SEQ ID NO: 236 - protein, 17 aa, synthetic construct
YIYNSGRTIYYADSVKG

SEQ ID NO: 237 - protein, 17 aa, synthetic construct
YITNSGRTIYYADSVKG

SEQ ID NO: 238 - protein, 17 aa, synthetic construct
YIANSGRTIYYADSVKG

SEQ ID NO: 239 - protein, 17 aa, synthetic construct
YISQSGRTIYYADSVKG

SEQ ID NO: 240 - protein, 17 aa, synthetic construct
YISNTGRTIYYADSVKG

SEQ ID NO: 241 - protein, 17 aa, synthetic construct
YISNYGRTIYYADSVKG

SEQ ID NO: 242 - protein, 17 aa, synthetic construct
YISNSGKTIYYADSVKG

SEQ ID NO: 243 - protein, 17 aa, synthetic construct
YISNSGRSIYYADSVKG

SEQ ID NO: 244 - protein, 17 aa, synthetic construct
YISNSARTIYYADSVKG

SEQ ID NO: 245 - protein, 17 aa, synthetic construct
YISNSSRTIYYADSVKG

SEQ ID NO: 246 - protein, 6 aa, synthetic construct
GWYMDA

SEQ ID NO: 247 - protein, 6 aa, synthetic construct
GYYMDA

SEQ ID NO: 248 - protein, 6 aa, synthetic construct
GLYMDA

SEQ ID NO: 249 - protein, 6 aa, synthetic construct
GIYMDA

SEQ ID NO: 250 - protein, 6 aa, synthetic construct
SFYMDA

SEQ ID NO: 251 - protein, 6 aa, synthetic construct
AFYMDA

SEQ ID NO: 252 - protein, 6 aa, synthetic construct
VFYMDA

SEQ ID NO: 253 - protein, 6 aa, synthetic construct
GHYMDA

SEQ ID NO: 254 - protein, 6 aa, synthetic construct
GWHMDA

SEQ ID NO: 255 - protein, 6 aa, synthetic construct
GWMMDA

SEQ ID NO: 256 - protein, 123 aa, Homo sapiens
DCCSCQEKWVGYRCNCYFISSEQKTWNESRHLCASQKSSLLQLQNTDELDFMSSSQQFYWIGLSYSEEHTAWLWENGSALSQYLFPSFETFNTKNCIAYNPNGNALDESCEDKNRYICKQQLI

SEQ ID NO: 257 - protein, 121 aa, Homo sapiens
ARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 258 - protein, 7 aa, synthetic construct
GSGGSGG

SEQ ID NO: 259 - protein, 251 aa, synthetic construct
DCCSCQEKWVGYRCNCYFISSEQKTWNESRHLCASQKSSLLQLQNTDELDFMSSSQQFYWIGLSYSEEHTAWLWENGSALSQYLFPSFETFNTKNCIAYNPNGNALDESCEDKNRYICKQQLIGSGGSGGARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 260 - protein, 231 aa, Homo sapiens
MSKQRGTFSEVSLAQDPKRQQRKPKGNKSSISGTEQEIFQVELNLQNPSLNHQGIDKIYDCQGLLPPPEKLTAEVLGIICIVLMATVLKTIVLIPFLEQNNSSPNTRTQKARHCGHCPEEWITYSNSCYYIGKERRTWEESLLACTSKNSSLLSIDNEEEMKFLASILPSSWIGVFRNSSHHPWVTINGLAFKHKIKDSDNAELNCAVLQVNRLKSAQCGSSMIYHCKHKL

SEQ ID NO: 261 - protein, 121 aa, Homo sapiens
ARHCGHCPEEWITYSNSCYYIGKERRTWEESLLACTSKNSSLLSIDNEEEMKFLASILPSSWIGVFRNSSHHPWVTINGLAFKHKIKDSDNAELNCAVLQVNRLKSAQCGSSMIYHCKHKL

SEQ ID NO: 262 - protein, 251 aa, synthetic construct
DCCSCQEKWVGYRCNCYFISSEQKTWNESRHLCASQKSSLLQLQNTDELDFMSSSQQFYWIGLSYSEEHTAWLWENGSALSQYLFPSFETFNTKNCIAYNPNGNALDESCEDKNRYICKQQLIGSGGSGGARHCGHCPEEWITYSNSCYYIGKERRTWEESLLACTSKNSSLLSIDNEEEMKFLASILPSSWIGVFRNSSHHPWVTINGLAFKHKIKDSDNAELNCAVLQVNRLKSAQCGSSMIYHCKHKL

SEQ ID NO: 263 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 264 - protein, 108 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKR

SEQ ID NO: 265 - protein, 121 aa, synthetic construct [sequence omitted]

SEQ ID NO: 266 - protein, 108 aa, synthetic construct
EIVMTQSPATLSLSPGERATLSCRASQSVSSYLAWYQQKPGQAPRLLIYDASNRATGIPARFSGSGSGTDFTLTISSLEPEDFAVYYCHQYGSTPLTFGGGTKAEIKR

SEQ ID NO: 267 - protein, 119 aa, synthetic construct [sequence omitted]

SEQ ID NO: 268 - protein, 108 aa, synthetic construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKR

SEQ ID NO: 269 - protein, 120 aa, synthetic construct [sequence omitted]

SEQ ID NO: 270 - protein, 108 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKR

SEQ ID NO: 271 - protein, 120 aa, synthetic construct
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSS

SEQ ID NO: 272 - protein, 108 aa, synthetic construct
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIKR

SEQ ID NO: 273 - protein, 124 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDHGSRHFWSYWGFDYWGQGTLVTVSS

SEQ ID NO: 274 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKR

SEQ ID NO: 275 - protein, 119 aa, synthetic construct [sequence omitted]

SEQ ID NO: 276 - protein, 108 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKR

SEQ ID NO: 277 - protein, 119 aa, synthetic construct
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSS

SEQ ID NO: 278 - protein, 107 aa, synthetic construct
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKR

SEQ ID NO: 279 - protein, 120 aa, synthetic construct [sequence omitted]

SEQ ID NO: 280 - protein, 108 aa, synthetic construct
DIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKR

SEQ ID NO: 281 - protein, 127 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARIGRYSYGLRSYYYYGMDVWGQGTTVTVSS

SEQ ID NO: 282 - protein, 108 aa, synthetic construct; identical to SEQ ID NO: 280

SEQ ID NO: 283 - protein, 113 aa, synthetic construct [sequence omitted]

SEQ ID NO: 284 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKR

SEQ ID NO: 285 - protein, 120 aa, synthetic construct [sequence omitted]

SEQ ID NO: 286 - protein, 112 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 287 - protein, 118 aa, synthetic construct [sequence omitted]

SEQ ID NO: 288 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 289 - protein, 119 aa, synthetic construct
EVQLLESGGGLVQPGGSLRLSCAASGFTFSSYIMMWVRQAPGKGLEWVSSIYPSGGITFADTVKGRFTISRDNSKNTLYLQMNSLRAEDTAVYYCARIKLGTVTTVDYWGQGTLVTVSS

SEQ ID NO: 290 - protein, 110 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 291 - protein, 121 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 292 - protein, 109 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKR

SEQ ID NO: 293 - protein, 121 aa, synthetic construct [sequence omitted]

SEQ ID NO: 294 - protein, 107 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKR

SEQ ID NO: 295 - protein, 122 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 296 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKR

SEQ ID NO: 297 - protein, 121 aa, synthetic construct
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVS

SEQ ID NO: 298 - protein, 107 aa, synthetic construct
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKR

SEQ ID NO: 299 - protein, 120 aa, synthetic construct
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVSS

SEQ ID NO: 300 - protein, 105 aa, synthetic construct
EIVLTQSPAIMSASPGERVTMTCSASSGVNYMHWYQQKPGTSPRRWIYDTSKLASGVPARFSGSGSGTDYSLTISSMEPEDAATYYCHQRGSYTFGGGTKLEIKR

SEQ ID NO: 301 - protein, 120 aa, synthetic construct; identical to SEQ ID NO: 299

SEQ ID NO: 302 - protein, 105 aa, synthetic construct
EIVLTQSPAIMSASPGERVTMTCSASSGVNYMHWYQQKPGTSPRRWIYDTSKLASGVPARFSGSGSGTSYSLTISSMEPEDAATYYCHQRGSYTFGGGTKLEIKR

SEQ ID NO: 303 - protein, 116 aa, synthetic construct [sequence omitted]

SEQ ID NO: 304 - protein, 108 aa, synthetic construct
DIQMTQSPSSVSASIGDRVTITCRASQGIDNWLGWYQQKPGKAPKLLIYDASNLDTGVPSRFSGSGSGTYFTLTISSLQAEDFAVYFCQQAKAFPPTFGGGTKVDIKG

SEQ ID NO: 305 - protein, 117 aa, synthetic construct
QVQLVQSGGGLVQPGGSLRLSCAASGFSFSTYAMSWVRQAPGKGLEWVSGISGSGGTTHYADSVKGRFTISRDNSKNTVNLQMNSLRAEDTAVYYCAKGLWFGEGLWGQGTLVTVSS

SEQ ID NO: 306 - protein, 113 aa, synthetic construct
DVVMTQSPLSLPVTLGQPASISCRSSQSLYYRSGYTFLDWYVQKPGQSPQLLIYQSSKRDSGVPDRISGSGSGTDFTLRISRVEAEDVGVYYCFQGTHWPYTFGQGTKLEIKR

SEQ ID NO: 307 - protein, 123 aa, synthetic construct
MEVQLVESGGGLVKPGGSLKLSCAASGFAFSIYDMSWVRQTPEKCLEWVAYISSGGGTTYYPDTVKGRFTISRDNAKNTLYLQMSSLKSEDTAMYYCARHSGYGTHWGVLFAYWGQGTLVTVS

SEQ ID NO: 308 - protein, 108 aa, synthetic construct
MDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGCGTKLEIK

SEQ ID NO: 309 - protein, 119 aa, synthetic construct
QLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSS

SEQ ID NO: 310 - protein, 113 aa, synthetic construct
DVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIKR

SEQ ID NO: 311 - protein, 122 aa, synthetic construct
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSS

SEQ ID NO: 312 - protein, 108 aa, synthetic construct
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKR

SEQ ID NO: 313 - protein, 116 aa, synthetic construct
VQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVS

SEQ ID NO: 314 - protein, 108 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKR

SEQ ID NO: 315 - protein, 125 aa, synthetic construct
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSS

SEQ ID NO: 316 - protein, 107 aa, synthetic construct
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 317 - protein, 486 aa, synthetic construct
DIQMTQSPSSLSASVGDRVTITCRASQSISSYLNWYQQKPGKAPKLLIYAASSLQSGVPSRFSGSGSGTDFTLTISSLQPEDFGVYYCQYYGISPPFTFGPGTKVDIKRGGGGSGGGGSGGGGSEVQLVESGGGLVKPGGSLRLSCAASGFTFSGYYMSWIRQAPGKGLEWLAYISNSGRTIYYADSVKGRFTISRDNSNSSLYLQMNSLRAEDTAVYYCARGFYMDAWGKGTTVTVSSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 318 - protein, 6 aa, synthetic construct
GSGGGS

SEQ ID NO: 319 - protein, 247 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGGGSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGSGGGS

SEQ ID NO: 320 - DNA, 741 bp, synthetic construct
  source 1..741: mol_type = other DNA
gatatacagatgacccaatcaccgtcttcactgtctgcctccgtgggggaccgcgtaaccatcacatgtaaagcatcacaagacgtctctatcggggtagcatggtaccaacagaagccgggtaaagcaccgaagttgttgatttattcagcatcttaccgatatacaggagtaccttccaggttctctggttcaggatcaggaacagatttcacactcaccatctcctcacttcagcccgaggactttgcaacgtactactgtcaacagtactatatttatccatatacctttggccaaggtaccaaggtagaaatcaagggaggtggaggttctgggggaggaggctcaggcggtggaggaagtgaagtacaactcgtagagtccgggggcggtttggtccagccgggtggtagtttgaggttgagctgcgcagcaagcggatttacttttaccgattatacaatggattgggtcagacaggcacccggcaagggtcttgagtgggtcgccgatgtgaaccccaatagtggaggaagtatttataatcaacgctttaaggggcgatttacattgtccgtagaccggtcaaaaaacactctttatctgcagatgaacagcttgcgagcggaggacacggctgtttattattgcgctaggaatctgggaccttccttctatttcgattattggggtcaggggaccttggtcactgtctctagtggaagcggcggaggatca

SEQ ID NO: 321 - protein, 248 aa, synthetic construct
DIQMTQSPSSLSASVGDRVTITCSASSSVTYMYWYQQKPGKAPKLLIYDTSNLASGVPSRFSGSGSGTDYTFTISSLQPEDIATYYCQQWSSHIFTFGQGTKVEIKGGGGSGGGGSGGGGSQVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYDGRYFDYWGQGTLVTVSSGSGGGS

SEQ ID NO: 322 - DNA, 744 bp, synthetic construct
  source 1..744: mol_type = other DNA
gacatacagatgactcagtccccgtctagcctgtctgcctccgttggggacagggtgactataacttgctccgcgagtagttctgttacttacatgtattggtatcaacagaaacccggtaaggcccccaagttgttgatttacgatacgtccaacttggcatctggggtcccctctcgcttcagtggaagtggctctggtacagactacacattcaccatctcctcattgcagccagaggacattgcaacttactactgccaacagtggtcctcccacattttcacttttggccagggcactaaggtagaaattaaaggtggaggagggagtggaggaggcggaagtggcggtgggggctctcaggtgcaacttgtgcagtcaggtgcagaggtcaaaaaaccaggcgcaagcgtgaaggtgagttgcaaggcaagcggttacacctttactagtcattggatgcattgggtgcgacaggcgcccggacaagggttggaatggattggagagttcaatcctagtaatgggcgcacaaattacaacgaaaagttcaagagtaaagcgacgatgactgtggatactagcacaaatacggcgtacatggaattgtcctctctcaggtccgaggacaccgcagtgtactattgcgccagtagggattatgactatgacgggcgatattttgattactggggtcagggaacattggtgaccgttagctcaggaagcggcggaggatca

SEQ ID NO: 323 - protein, 108 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGLTAGSGGGS

SEQ ID NO: 324 - DNA, 324 bp, synthetic construct
  source 1..324: mol_type = other DNA
aagaaagtagtttatggcaagaaaggcgataccgttgagttgacatgcacggcttcacaaaagaagaatattcagtttcattggaaaaatagcaaccaaataaagattttgggtaaccagggctcatttctgacgaagggaccctccaaactcaatgaccgagttgacagtaggcggtctctctgggaccagggaaattttcctctgataattaaaaacctcaaacctgaggattcagatacatatatatgtgaggtggaggatcaaaaggaagaggtccagttggttgtcgtagggctgacagccggaagcggcggaggatca

SEQ ID NO: 325 - protein, 9 aa, synthetic construct
VMAPRTLVL

SEQ ID NO: 326 - protein, 6 aa, synthetic construct
GSGGGG